(19) United States
(12) Patent Application Publication
(10) Pub. No.: US 2023/0285357 A1
(43) Pub. Date: Sep. 14, 2023

(54) COMPOSITIONS AND METHODS FOR TRANSDIFFERENTIATING CELLS

(71) Applicant: The Regents of the University of California, Oakland, CA (US)

(72) Inventors: Yucheng Yao, Los Angeles, CA (US); Kristina I Bostrom, Los Angeles, CA (US); Jiayi Yao, Los Angeles, CA (US)

(21) Appl. No.: 17/927,485

(22) PCT Filed: May 26, 2021
(86) PCT No.: PCT/US2021/034204

§ 371 Date: Nov. 23, 2022

Related U.S. Application Data
(60) Provisional application No. 63/030,626, filed on May 27, 2020.

(51) Int. Cl. A61K 31/404; C12N 15/113; A61P 9/00

(52) U.S. Cl. CPC A61K 31/404; C12N 15/113; C12N 15/1137; A61P 9/00; C12N 2310/14

(57) ABSTRACT

Provided herein are methods of treating or preventing vascular calcification in a subject in need thereof by administering to the subject an agent that inhibits the activity of or decreases the levels of glycogen synthase kinase 3 (GSK3), an agent that inhibits the activity of or decreases the levels of SMAD1, and/or an agent that activates or increases the levels of β-catenin.

### RELATED APPLICATIONS

This application claims the benefit of priority to U.S. Provisional Application Ser. No. 63/030,626, filed on May 27, 2020, the entire contents of which are incorporated herein in its entirety by this reference.

### REFERENCE TO A SEQUENCE LISTING

This application contains a Sequence Listing in computer readable form. The computer readable form is incorporated herein by reference. Said ASCII copy, created on May 24, 2023, is named UCH-23801_SL.txt and is 69,640 bytes in size.

### BACKGROUND

Transitions between cell fates contribute to the normal developmental process, but ill-fated transitions initiate pathological processes. It remains unknown if shifting ill-fated cells back to normal differentiation can treat disease. One of severe medical condition caused by ill-fated cell transition is vascular calcification. Vascular calcification is an active process involving ectopic bone formation, where osteogenic differentiation occurs in cells transited from other lineages. Vascular endothelium undergoes endothelial-mesenchymal transitions to contributes cells to vascular calcification. In this dramatic switch of cell fates, endothelial differentiation decreases, mesenchymal differentiation emerges, and endothelial cells (ECs) gain plasticity toward osteoblast-like cells.

### SUMMARY

Provided herein are methods of treating or preventing vascular calcification in a subject in need thereof by administering to the subject an agent that inhibits the activity of or decreases the levels of glycogen synthase kinase 3 (GSK3), an agent that inhibits the activity of or decreases the levels of SMAD1, and/or an agent that activates or increases the levels of β-catenin. Also provided herein are methods of inducing or increasing osteoblastic-endothelial transdifferentiation in a subject in need thereof comprising administering to the subject an agent that inhibits the activity of or decreases the levels of glycogen synthase kinase 3 (GSK3), an agent that inhibits the activity of or decreases the levels of SMAD1, and/or an agent that activates or increases the levels of β-catenin. Provided herein are methods of inhibiting or decreasing osteogensis in a subject in need thereof by administering to the subject an agent that inhibits the activity of or decreases the levels of glycogen synthase kinase 3 (GSK3), an agent that inhibits the activity of or decreases the levels of SMAD1, and/or an agent that activates or increases the levels of β-catenin. The GSK3 may be GSK33.

Also provided herein are methods of treating or preventing a condition in a subject by administering to the subject an agent that modulates the activity of glycogen synthase kinase 3 (GSK3), an agent that inhibits the activity of or decreases the levels of SMAD1, and/or an agent that activates or increases the levels of β-catenin. One agent or multiple agents may be administered to subjects.

The agent described herein may be a small molecule, such as SB216763. The agent may be a polypeptide. The agent may be an inhibitory polynucleotide specific for an GSK3 protein. The GSK3 may be GSK33. The inhibitory polynucleotide is selected from siRNA, shRNA, and an antisense RNA molecule, or a polynucleotide that encodes a molecule selected from siRNA, shRNA, and/or an antisense RNA molecule.

The agent provided herein may be an intrabody. An intrabody is an antibody that has been designed to be expressed intracellularly and can be directed to a specific target present in various subcellular locations.

The agent may be an inhibitory polynucleotide specific for an SMAD1 protein. The inhibitory polynucleotide may be selected from siRNA, shRNA, and an antisense RNA molecule, or a polynucleotide that encodes a molecule selected from siRNA, shRNA, and/or an antisense RNA molecule.

The agent may be a polynucleotide encoding a β-catenin protein. Provided herein are methods of treating or preventing a condition in a subject, comprising administering to the subject an agent that modulates the activity of SMAD1. The agent described herein may be a small molecule, such as SB216763. The agent may be a polypeptide. The agent may be an inhibitory polynucleotide specific for a GSK3 protein. The GSK3 may be GSK33. The agent may be an agent is an inhibitory polynucleotide specific for an SMAD1 protein.

The inhibitory polynucleotide is selected from siRNA, shRNA, and an antisense RNA molecule, or a polynucleotide that encodes a molecule selected from siRNA, shRNA, and/or an antisense RNA molecule.

The methods herein also encompass methods of treating or preventing a condition in a subject, comprising administering to the subject an agent that activates or increases the levels of β-catenin. The agent may be a polynucleotide encoding a β-catenin peptide.

The condition described herein may be cardiovascular disease, chronic kidney disease, diabetes mellitus, or fibrodysplasia ossificans progressiva.

Also provided herein are methods of determining whether a test agent is an inhibitor of vascular calcification, comprising a) forming a test mixture comprising: a test agent, wherein the test agent is a polynucleotide, a small molecule, or a peptide; b) contacting the test mixture with cells expressing GSK3; wherein a test agent that inhibits the activity of or decreases the levels of GSK3 compared to the activity of or level of GSK3 in a control mixture is an inhibitor of vascular calcification.

The GSK3 may be is GSK33. In some embodiments, the test agent is linked to a detectable moiety. In some embodiments, the GSK3 is linked to a detectable moiety.

Also provided herein are methods of determining whether a test agent is an inhibitor of vascular calcification, comprising: a) forming a test mixture comprising: a test agent, wherein the test agent is a polynucleotide, a small molecule, or a peptide; b) contacting the test mixture with cells expressing SMAD1; wherein a test agent that inhibits the activity of or decreases the levels of SMAD1 compared to the activity of or level of SMAD1 in a control mixture is an inhibitor of vascular calcification. In some embodiments, the test agent is linked to a detectable moiety. In some embodiments, the SMAD1 is linked to a detectable moiety. The test agent may be a peptide, small molecule, or inhibitory polynucleotide. Also provided herein are methods of determining whether a test agent is an inhibitor of vascular calcification, comprising: a) forming a test mixture comprising: a test agent, wherein the test agent is a polynucleotide or a peptide; b) contacting the test mixture with cells expressing β-catenin; wherein a test agent that increases the activity of or the levels of β-catenin compared to the activity of or level of β-catenin in a control mixture is an inhibitor of vascular calcification. The agent may be a polynucleotide encoding a β-catenin peptide. In some embodiments, the control mixture is substantially identical to the test mixture except that the control mixture does not comprise a test agent. In other embodiments, the test agent is a member of a library of test agents.

In some aspects provided herein are methods of treating or preventing vascular calcification in a subject in need thereof, comprising administering to the subject a test agent identified using the methods provided herein. In some aspects, provided herein are methods of inducing or increasing osteoblastic-endothelial transdifferentiation in a subject in need thereof, comprising administering to the subject a test agent identified using the methods provided herein. Laos provided herein are methods of decreasing or inhibiting osteogenesis in a subject in need thereof, comprising administering to the subject a test agent identified using the methods provided herein. In some aspects, the subject has a condition, and the condition may be cardiovascular disease, chronic kidney disease, diabetes mellitus or fibrodysplasia ossificans progressiva. In some embodiments, the methods herein provide for administering two or more, three or more, four or more, five or more, six or more, or seven or more of the agents provided herein.

### BRIEF DESCRIPTION OF THE DRAWINGS

FIG. 1 has eight parts, A-H, and shows high throughput screening identified SB216763 as an inducer of osteoblastic-endothelial transdifferentiation. Part A shows a strategic drawing osteoblastic-endothelial transdifferentiation affecting vascular calcification. Part B shows a high throughput screening that identified SB216763 as an inducer of eGFP expression in Flk1-eGFP osteoblasts as indicated by the black arrow. Part C shows morphology of Flk1-eGFP osteoblasts after treatment with SB216763 (n=9). Scale bar, 50 μm. Part D shows FACS analysis of SB216763-treated osteoblasts using anti-CD31 and anti-VE-cadherin (VE-cad) antibodies (n=8). Part E shows expression of the osteogenic markers Cbfa1, osterix (OSX), and osteocalcin (OC) and the endothelial markers CD31, VE-cadherin, CD34, eNOS in osteoblasts treated with different does of SB216763 (n=8). Part F shows time-course expression of the osteogenic markers Cbfa1, osterix (OSX), and osteocalcin (OC) and the endothelial markers CD34, VE-cadherin, CD31, and eNOS in SB216763-treated osteoblasts (n=8). Part G shows heatmap and GO analysis of a cohort of genes with decreased expression in SB216763-treated osteoblasts. 827 genes decreased in expression in SB21673-treated osteoblasts were overlapped with 1357 genes identified as low expression when compared osteoblast vs EC, and identified a 307-gene cohort with decreased expression in both analyses. Part H shows heatmap and GO analysis of a cohort of genes with increased expression in SB216763-treated osteoblasts. 519 genes with increased expression after SB21673 treatment were overlapped with 1801 genes with high expression when compared EC vs osteoblast, and identified another 235 genes that showed increased expression in both analyses.

FIG. 2 has eleven parts, A-K, and shows SB216763 treatment causes osteoblasts to lose osteogenic capacity but gain endothelial function. Part A shows a schematic of experimental procedure for ectopic bone formation in vivo. HA/TCP, hydroxyapatite/tricalcium phosphate. Part B shows MicroCT images of ectopic bone formation after cell transplantation (n=6). Scale bar, 5 mm. PBS, phosphate buffered saline. Part C shows relative volume of bone formation (n=6). Part D shows implants of ectopic bone collected from the mice after cell transplantation (n=6). Part E shows H&E staining of implants (n=6). Black arrows indicate osteocytes. Scale bar, 50 μm. Part F shows a schematic of experimental procedure for cell transplantation using the hindlimb ischemia model. Part G shows laser doppler perfusion images after cell transplantation (n=8). Top, measurement camera. Bottom, documentation camera. PBS, phosphate buffered saline. HAEC, human aortic endothelial cells. Scare bar, 10 mm. Part H shows percentage of blood flow perfusion after cell transplantation normalized by perfusion of normal limb (n=6). Part I shows immunostaining of new vessels after cell transplantation using anti-CD31 antibodies (n=10). Scale bar, 50 μm. Part J shows eGFP positive cells (green) were observed in the endothelium of new vessels, which stained with anti-vWF antibodies (red). Part K shows analysis of vascular density in ischemic sites after cell transplantation (n=10).

FIG. 3 has ten parts, A-J, and shows increased β-catenin decreases SMAD1 and together are responsible for SB216763 to induce osteoblastic-endothelial transdifferentiation. Part A shows a schematic of decreased SMAD1 combined with increased β-catenin caused by GSK3 inhibition converting osteoblasts into endothelial-like cells. Part B shows immunoblotting of SMAD1, phosphorylation (p) SMAD1 and β-catenin in osteoblasts treated with different doses of SB216763 (n=6). Part C shows immunoblotting of SMAD1 and in osteoblasts transfected with β-catenin siRNA in accompany with 10 μM SB216763 with (n=6). SCR, scrambled siRNA. Part D shows DNA-binding sites of β-catenin in promoter region of Smad1 gene. Parts E and F show a ChIP assay of DNA-binding of β-catenin and histone modification in promoter of Smad1 gene. Part G shows microCT images of ectopic bone formation and relative volume of bone formation in the implants after cell transplantation. Osteoblasts were used as controls. SB216763, SB216763-treated osteoblasts. SB216763/CMV-SMAD1, SB216763-treated osteoblasts infected with lentiviral vectors containing CMV promoter-driven SMAD1 cDNA. SB216763/β-catenin si, SB216763-treated osteoblasts infected with lentiviral vectors containing β-catenin siRNA (si) (n=6). Scale bar, 5 mm. Part F shows that H&E staining of the sections of implants (n=6). Black arrows indicate osteocytes. Scale bar, 50 μm. Part I shows laser doppler perfusion images after cell transplantation (n=8). Osteoblasts were used as controls. HAEC, human aortic endothelial cells. SB216763, SB216763-treated osteoblasts. SCR, scrambled siRNA. SB216763/β-catenin si, SB216763-treated osteoblasts infected with lentiviral vectors containing β-catenin siRNA (si). SB216763/CMV-SMAD1, SB216763-treated osteoblasts infected with lentiviral vectors containing CMV promoter-driven SMAD1 cDNA. (n=6). Scare bar, 10 mm. Part G shows the percentage of blood flow perfusion after cell transplantation normalized by perfusion of normal limb (n=6).

FIG. 4 has two parts, A-B and shows modulation of β-catenin and SMAD1 alter transcriptional landscapes of osteoblasts. Part A shows a heatmap of the cohorts of genes with decreased SMAD1 DNA-binding and GO analysis of the genes with decreased SMAD1 DNA-binding and decreased expression in SB21673-treated osteoblasts. Part B shows a heatmap of the cohorts of genes with increased β-catenin DNA-binding (bottom) in SB21673-treated osteoblasts and GO analysis of the genes with increased β-catenin DNA-binding and increased expression in SB21673-treated osteoblasts.

FIG. 5 has eleven parts, A-K, and shows that SB216763 reverses osteogenesis to endothelial differentiation to ameliorate vascular calcification in Mgp^{-/-} mice. Part A shows a schematic of experimental procedure to determine the effect SB216763 treatment in early calcification. Part B shows Von Kossa staining of aortic tissues (n=7). Scale bar, 50 μm. Part C shows total aortic calcium after SB216763 treatment (n=8). Part D shows FACS analysis of CD31+ aortic cells using anti-osterix (OSX) and anti-VE-cadherin antibodies (n=6). Part E shows that immunoblotting of aortic tissues of Mgp^{-/-} mice after SB216763 treatment (n=6). Part F shows schematic of experimental procedure to examine the effect of SB216763 treatment in late calcification. Parts G and H shows microCT images of aortic calcification in Mgp^{-/-} mice after SB216763 treatment (n=6). Scale bar, 5 mm. Part I shows total aortic calcium and calcification score in Mgp^{-/-} mice after SB216763 treatment (n=8). Part J shows H&E staining of Mgp^{-/-} aortic tissues after SB216763 treatment (n=6). Part K shows FACS analysis of Mgp^{-/-} CD31+ aortic cells using anti-osterix (OSX) and anti-VE-cadherin antibodies (n=6).

FIG. 6 has seven parts, A-G, and shows that osteoblastic lineage tracing reveals the shift of osteoblast-like cells to endothelial differentiation in calcified aortic tissue. Part A shows a schematic of experimental procedure to characterize the shift of osteoblast-like cells toward endothelial differentiation. Part B shows FACS analysis of GFP+CD31+ aortic cells of Osx-GfptgMgp^{-/-} mice treated with or without SB216763 (left). The cells are confirmed to express GFP after isolation (right) (n=5). Scale bar, 50 μm. Part C shows microCT images of ectopic bone formation and analysis of relative volume of bone formation after cell transplantation (n=6). Scale bar, 5 mm. Part D shows H&E staining of implants (n=6). Black arrows indicate osteocytes. Scale bar, 50 μm. Part E shows a laser doppler perfusion images after cell transplantation (n=6). Top, measurement camera. Bottom, documentation camera. Scare bar, 10 mm. Part F shows percentage of blood flow perfusion after cell transplantation normalized by perfusion of normal limb (n=6). Part G shows analysis of vascular density in ischemic sites after cell transplantation (n=10).

FIG. 7 has nine parts, A-I, and shows specific gene deletion of GSK3β in osteoblasts reverses osteogenic-endothelial transdifferentiation to ameliorate vascular calcification in Mgp^{-/-} mice. Part A shows immunoblotting of osteoblasts transfected with GSK3α or GSK3β siRNA (n=6). B-actin was used as loading control. Part B shows real-time PCR showing the expression of Cbfa1, osterix (OSX), VE-cadherin (VE-cad) and CD31 in osteoblasts transfected with GSK3α or GSK3β siRNA (n=6). Part C shows a schematic of experimental procedure. Part D shows expression of GSK3β in aortic tissues (n=8). Part E shows a total aortic calcium of Col1α1^{creERT2}GSK3β^{flox/flox}Mgp^{-/-} mice (n=8) with or without injection of tamoxifen. f/f, flox/flox. Part F and G shows microCT images and aortic calcification score of Col1α1^{creERT2}GSK3β^{flox/flox}Mgp^{-/-} mice with or without injection of tamoxifen (n=6). Scale bar, 5 mm. Part H shows H&E staining of Col1α1^{creERT2}GSK3β^{flox/flow}Mgp^{-/-} aortic tissues with or without injection of tamoxifen (n=8). Scale bar, 5 mm. Part I shows CD31+ aortic cells isolated from Col1α1^{creERT2}GSK3β^{flow/flox}Mgp^{-/-} aortic tissues with or without injection of tamoxifen and analyzed by FACS analysis using anti-osterix (OSX) and anti-VE-cadherin antibodies (n=6).

FIG. 8 has three parts, A-C, and shows that SB216763 treatment has no effect on bone formation. Part A shows that micro-CT imaging of bone tissues of mice treated with or without SB216763 (n=6). Scale bar, 1 mm. Part B shows relative bone volume and connectivity density of bone tissues of mice treated with or without SB216763 (n=6). Part C shows immunostaining of bone tissues by using anti-CD31 or anti-osterix antibodies (n=6). Scale bar, 50 μm.

FIG. 9 has six parts, A-F, and shows that SB216763 induces endothelial differentiation in human osteoblast but does not activate other lineages. Part A shows high throughput screening identified SB216763 as an inducer of eGFP expression in Flk1-eGFP osteoblasts as indicated by the black arrow. Part B shows a schematic drawing of GSK3 inhibition inducing osteoblastic-endothelial transdifferentiation. Part C shows expression of the osteogenic markers Cbfa1, osterix (OSX), and osteocalcin (OC) and the endothelial markers CD31, VE-cadherin, CD34, and eNOS in human osteoblasts treated with different doses of SB216763 (n=8). Part D shows time-course expression of the osteogenic markers Cbfa1, osterix (OSX), and osteocalcin (OC) and the endothelial markers CD34, VE-cadherin, CD31, and eNOS in SB216763-treated human osteoblasts (n=8). Part E shows expression of markers for different lineages examined by real-time PCR. The lineages included mesenchymal and stem cell, smooth muscle cell, fibroblast, pericyte, adipocyte, pulmonary, hepatic, neuronal, cardiac, hematopoietic and renal lineages (n=6). α-SMA, alpha smooth muscle actin. MYH11, myosin heavy chain 11. FSP, fibroblast-specific protein. NG2, neuron-glial antigen 2. Spb, surfactant protein b. CCSP, club-cell secretory protein. Aqp5, aquaporin 5. Tnc, troponin c. ACTC1, actin alpha cardiac muscle 1. MYH7, myosin heavy chain 7. AP2, adipocyte protein 2. C/EBP, CCAAT/enhancer binding protein. Part F shows expression of representative genes in glucose metabolism (n=6). CK2, casein kinase 2. GLUT4, glucose transporter 4. IRS1 and 2, insulin receptor substrates 1 and 2.

FIG. 10 has three parts, A-C, and shows SB216763 treatment causes osteoblasts to lose the osteogenic capacity but gain endothelial function. Part A shows osteogenesis assay of SB216763-treated osteoblasts stained by von Kossa (n=5). Scale bar, 50 μm. Part B shows immunostaining of the sections of implants with anti-osterix antibodies (n=6). Scale bar, 50 μm. Part C shows a tube formation assay of SB216763-treated osteoblasts (n=5). Scale bar, 50 μm.

FIG. 11 has four parts, A-D, and shows decreased SMAD1 and increased β-catenin are responsible for the SB216763-induction of osteoblastic-endothelial transdifferentiation. Part A shows expression of SMAD1 or β-catenin in SB216763-treated osteoblasts infected with lentiviral vectors containing CMV promoter-driven SMAD1 cDNA or infected with lentiviral vectors containing β-catenin siRNA. (n=6). Part B shows osteogenesis assay of SB21763-treated osteoblasts infected with lentiviral vectors containing CMV promoter-driven SMAD1 cDNA or infected with lentiviral vectors containing β-catenin siRNA. (n=6). Scale bar, 50 μm. Part C shows immunostaining of sections of implants with anti-osterix antibodies (n=6). Scale bar, 50 μm. Part D shows a tube formation assay of SB21763-treated osteoblasts infected with lentiviral vectors containing CMV promoter-driven SMAD1 cDNA or containing β-catenin siRNA (n=6)

FIG. 12 has four parts, A-D, and shows SB216763 reverses osteogenesis to endothelial differentiation to ameliorate vascular calcification in Mgp^{-/-} mice. Part A shows microCT images of aortic calcification in Mgp^{-/-} and wild type (Mgp^{+/+}) mice after SB216763 treatment (n=6). Scale bar, 5 mm. Part B shows alizarin red staining of aortic tissues (n=6). Scale bar, 5 mm. Part C shows H&E staining of aortic tissues of Mgp^{-/-} and Mgp^{+/+} mice after SB216763 treatment (n=6). Scale bar, 50 μm. Part D shows expression of cbfa1 and osteopontin (OPN) in aortic tissues of Mgp^{-/-} and Mgp^{+/+} mice after SB216763 treatment (n=8).

FIG. 13 has three parts, A-C, and shows specific deletion of GSK3β in osteoblasts reduces vascular calcification. Part A shows microCT images of mice after injection of tamoxifen (n=6). Scale bar, 5 mm. Part B shows H&E staining of aortic tissues of mice after injection of tamoxifen (n=8). Scale bar, 5 mm. Part C shows expression of Cbfa1 and osteopontin (OPN) in aortic tissues of mice after injection of tamoxifen (n=8).

### DETAILED DESCRIPTION

The present disclosure is related, in part, to the discovery that GSK3 inhibition switches the osteoblastic fate for endothelial differentiation by modulating SMAD1 and β-catenin, and this switch of cell fates improves vascular calcification. This disclosure provides a new approach for osteoblastic-endothelial transdifferentiation. Vascular calcification is the pathological deposition of mineral in the vascular system. Vascular calcification is highly associated with cardiovascular disease mortality, particularly in patients with diabetes and chronic kidney diseases (CKD). Vascular calcification may also be associated with other disorders, such as fibrodysplasia ossificans progressiva. Fibrodysplasia ossificans progressiva is a disorder in which muscle tissue and connective tissue such as tendons and ligaments are gradually replaced by bone therefore forming bone outside the skeleton that constrains movement.

Provided herein are methods of treating or preventing vascular calcification in a subject in need thereof by administering to the subject an agent that inhibits the activity of or decreases the levels of glycogen synthase kinase 3 (GSK3), an agent that inhibits the activity of or decreases the levels of SMAD1, and/or an agent that activates or increases the levels of β-catenin. Also provided herein are methods of inducing or increasing osteoblastic-endothelial transdifferentiation in a subject in need thereof comprising administering to the subject an agent that inhibits the activity of or decreases the levels of glycogen synthase kinase 3 (GSK3), an agent that inhibits the activity of or decreases the levels of SMAD1, and/or an agent that activates or increases the levels of β-catenin. Provided herein are methods of inhibiting or decreasing osteogensis in a subject in need thereof by administering to the subject an agent that inhibits the activity of or decreases the levels of glycogen synthase kinase 3 (GSK3), an agent that inhibits the activity of or decreases the levels of SMAD1, and/or an agent that activates or increases the levels of β-catenin.

Also provided herein are methods of treating or preventing a condition in a subject by administering to the subject an agent that modulates the activity of glycogen synthase kinase 3 (GSK3), an agent that inhibits the activity of or decreases the levels of SMAD1, and/or an agent that activates or increases the levels of β-catenin. One agent or multiple agents may be administered to subjects.

### Definitions

For convenience, certain terms employed in the specification, examples, and appended claims are collected here.

As used herein the specification, “a” or “an” may mean one or more. As used herein in the claim(s), when used in conjunction with the word “comprising”, the words “a” or “an” may mean one or more than one. As used herein “another” may mean at least a second or more.

As used herein, the term “administering” means providing a pharmaceutical agent or composition to a subject, and includes, but is not limited to, administering by a medical professional and self-administering.

The term “agent” is used herein to denote a chemical compound, a small molecule, a mixture of chemical compounds and/or a biological macromolecule (such as a nucleic acid, an antibody, an antibody fragment, a small molecule, a protein or a peptide). Agents may be identified as having a particular activity by screening assays described herein below. The activity of such agents may render them suitable as a “therapeutic agent” which is a biologically, physiologically, or pharmacologically active substance (or substances) that acts locally or systemically in a subject.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing.

As used herein, the term “antibody” may refer to both an intact antibody and an antigen binding fragment thereof. Intact antibodies are glycoproteins that include at least two heavy (H) chains and two light (L) chains inter-connected by disulfide bonds. Each heavy chain includes a heavy chain variable region (abbreviated herein as V_{H}) and a heavy chain constant region. Each light chain includes a light chain variable region (abbreviated herein as V_{L}) and a light chain constant region. The V_{H} and V_{L} regions can be further subdivided into regions of hypervariability, termed complementarity determining regions (CDR), interspersed with regions that are more conserved, termed framework regions (FR). The term “antibody” includes, for example, monoclonal antibodies, polyclonal antibodies, chimeric antibodies, humanized antibodies, human antibodies, multispecific antibodies (e.g., bispecific antibodies), single-chain antibodies and antigen-binding antibody fragments. Any antibody disclosed herein may be specific for GSK3 and modulate the activity of GSK3. Any antibody disclosed herein may be specific for SMAD1 and modulate the activity of SMAD1. Any antibody disclosed herein may be specific for β-catenin and modulate the activity of β-catenin.

The terms “antigen binding fragment” and “antigen-binding portion” of an antibody, as used herein, refers to one or more fragments of an antibody that retain the ability to bind to an antigen. Examples of binding fragments encompassed within the term “antigen-binding fragment” of an antibody include Fab, Fab′, F(ab′)₂, Fv, scFv, disulfide linked Fv, Fd, diabodies, single-chain antibodies, and other antibody fragments that retain at least a portion of the variable region of an intact antibody. These antibody fragments can be obtained using conventional recombinant and/or enzymatic techniques and can be screened for antigen binding in the same manner as intact antibodies.

As used herein, the term “monoclonal antibody” refers to an antibody obtained from a population of substantially homogeneous antibodies that specifically bind to the same epitope, i.e., the individual antibodies comprising the population are identical except for possible naturally occurring mutations that may be present in minor amounts. The modifier “monoclonal” indicates the character of the antibody as being obtained from a substantially homogeneous population of antibodies, and is not to be construed as requiring production of the antibody by any particular method.

The terms “polynucleotide”, and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified, such as by conjugation with a labeling component. The term “recombinant” polynucleotide means a polynucleotide of genomic, cDNA, semisynthetic, or synthetic origin which either does not occur in nature or is linked to another polynucleotide in a non-natural arrangement.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body.

The term “preventing” is art-recognized, and when used in relation to a condition, such as a local recurrence, is well understood in the art, and includes administration of a composition which reduces the frequency of, or delays the onset of, symptoms of a medical condition in a subject relative to a subject which does not receive the composition. Thus, prevention of acne includes, for example, reducing the number of detectable acne lesions in a population of patients receiving a prophylactic treatment relative to an untreated control population, and/or delaying the appearance of detectable lesions in a treated population versus an untreated control population, e.g., by a statistically and/or clinically significant amount.

The term “small molecule” is a term of the art and includes molecules that are less than about 1000 molecular weight or less than about 500 molecular weight. In one embodiment, small molecules do not exclusively comprise peptide bonds. In another embodiment, small molecules are not oligomeric. Exemplary small molecule compounds which can be screened for activity include, but are not limited to, peptides, peptidomimetics, nucleic acids, carbohydrates, small organic molecules (e.g., polyketides) (Cane et al. (1998) Science 282:63), and natural product extract libraries. In another embodiment, the compounds are small, organic non-peptidic compounds. In a further embodiment, a small molecule is not biosynthetic.

As used herein, the term “subject” means a human or non-human animal selected for treatment or therapy.

A “therapeutically effective amount” of a compound with respect to the subject method of treatment refers to an amount of the compound(s) in a preparation which, when administered as part of a desired dosage regimen (to a mammal, preferably a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

As used herein, the term “treating” or “treatment” includes reversing, reducing, or arresting the symptoms, clinical signs, and underlying pathology of a condition in a manner to improve or stabilize a subject's condition.

### Modulators of GSK3, SMAD1, and β-Catenin

The agents disclosed herein may induce or increase osteoblastic-endothelial transdifferentiation, decrease or inhibit vascular calcification, and/or inhibiting or decreasing osteogenesis in a subject in need thereof. Also provided herein are methods which comprise administering the agents disclosed herein to subjects afflicted with a disease or condition disclosed herein.

### Small Molecule Agents

Small molecule agents useful in the methods disclosed herein include those known in the art and those identified using the screening assays described herein. For example, in some embodiments the agent is a GSK3 inhibitor (e.g., a GSK3α and/or GSK3β inhibitor). Examples of GSK3 inhibitors include, but are not limited to, lithium chloride (LiCl), maleimide derivatives (e.g., SB216763, Indolyl-maleimide inhibitors, 3-anilino-4-arylmaleimides 1-3, or bisindolyl maleimide and benzofuranyl-indolyl maleimide inhibitors), staurosporine and organometallic inhibitors, indole derivatives, paullone derivatives, pyrazolamide derivatives, pyrimidine and furopyrimidine derivatives, oxadiazole derivatives, and thiazole derivatives, and pharmaceutically acceptable salts thereof. The agent may be a small molecule inhibitor of SMAD1. An exemplary inhibitory small molecule of SMAD 1 includes myvicein. Screens for inhibitors of SMAD1 can be found in U.S. Pat. No. 6,998,240B2, hereby incorporated by reference in its entirety. The agent may be a small molecule activator of β-catenin, such as CHIR-99021 (CT99021), methyl vanillate, or Wnt agonist 1. Additional small molecule activators can be found in US20170049793A1, hereby incorporated by reference in its entirety.

Agents useful in the methods disclosed herein may be obtained from any available source, including systematic libraries of natural and/or synthetic compounds. Agents may also be obtained by any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann et al., 1994, J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, 1997, Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of agents may be presented in solution (e.g., Houghten, 1992, Biotechniques 13:412-421), or on beads (Lam, 1991, Nature 354:82-84), chips (Fodor, 1993, Nature 364:555-556), bacteria and/or spores, (Ladner, U.S. Pat. No. 5,223,409), plasmids (Cull et al, 1992, Proc Natl Acad Sci USA 89:1865-1869) or on phage (Scott and Smith, 1990, Science 249:386-390; Devlin, 1990, Science 249:404-406; Cwirla et al, 1990, Proc. Natl. Acad. Sci. 87:6378-6382; Felici, 1991, J. Mol. Biol. 222:301-310; Ladner, supra.).

Agents useful in the methods disclosed herein may be identified, for example, using assays for screening candidate or test compounds which inhibit complex formation between a receptor provided herein and a ligand described herein.

### Interfering Nucleic Acid Agents

In certain embodiments, interfering nucleic acid molecules that selectively target GSK3 and/or SMAD1 and/or used in methods described herein. Interfering nucleic acids generally include a sequence of cyclic subunits, each bearing a base-pairing moiety, linked by intersubunit linkages that allow the base-pairing moieties to hybridize to a target sequence in a nucleic acid (typically an RNA) by Watson-Crick base pairing, to form a nucleic acid: oligomer heteroduplex within the target sequence. Interfering RNA molecules include, but are not limited to, antisense molecules, siRNA molecules, single-stranded siRNA molecules, miRNA molecules and shRNA molecules.

Typically at least 17, 18, 19, 20, 21, 22 or 23 nucleotides of the complement of the target mRNA sequence are sufficient to mediate inhibition of a target transcript. Exemplary mRNA target sequences are included in Table 1. Perfect complementarity is not necessary. In some embodiments, the interfering nucleic acid molecule is double-stranded RNA. The double-stranded RNA molecule may have a 2 nucleotide 3′ overhang. In some embodiments, the two RNA strands are connected via a hairpin structure, forming a shRNA molecule. shRNA molecules can contain hairpins derived from microRNA molecules. For example, an RNAi vector can be constructed by cloning the interfering RNA sequence into a pCAG-miR30 construct containing the hairpin from the miR30 miRNA. RNA interference molecules may include DNA residues, as well as RNA residues.

Interfering nucleic acid molecules provided herein can contain RNA bases, non-RNA bases or a mixture of RNA bases and non-RNA bases. For example, interfering nucleic acid molecules provided herein can be primarily composed of RNA bases but also contain DNA bases or non-naturally occurring nucleotides.

The interfering nucleic acids can employ a variety of oligonucleotide chemistries. Examples of oligonucleotide chemistries include, without limitation, peptide nucleic acid (PNA), linked nucleic acid (LNA), phosphorothioate, 2′O-Me-modified oligonucleotides, and morpholino chemistries, including combinations of any of the foregoing. In general, PNA and LNA chemistries can utilize shorter targeting sequences because of their relatively high target binding strength relative to 2′O-Me oligonucleotides. Phosphorothioate and 2′O-Me-modified chemistries are often combined to generate 2′O-Me-modified oligonucleotides having a phosphorothioate backbone. See, e.g., PCT Publication Nos. WO/2013/112053 and WO/2009/008725, incorporated by reference in their entireties.

Peptide nucleic acids (PNAs) are analogs of DNA in which the backbone is structurally homomorphous with a deoxyribose backbone, consisting of N-(2-aminoethyl) glycine units to which pyrimidine or purine bases are attached. PNAs containing natural pyrimidine and purine bases hybridize to complementary oligonucleotides obeying Watson-Crick base-pairing rules, and mimic DNA in terms of base pair recognition (Egholm, Buchardt et al. 1993). The backbone of PNAs is formed by peptide bonds rather than phosphodiester bonds, making them well-suited for antisense applications (see structure below). The backbone is uncharged, resulting in PNA/DNA or PNA/RNA duplexes that exhibit greater than normal thermal stability. PNAs are not recognized by nucleases or proteases.

Despite a radical structural change to the natural structure, PNAs are capable of sequence-specific binding in a helix form to DNA or RNA. Characteristics of PNAs include a high binding affinity to complementary DNA or RNA, a destabilizing effect caused by single-base mismatch, resistance to nucleases and proteases, hybridization with DNA or RNA independent of salt concentration and triplex formation with homopurine DNA. PANAGENE™ has developed its proprietary Bts PNA monomers (Bts; benzothiazole-2-sulfonyl group) and proprietary oligomerization process. The PNA oligomerization using Bts PNA monomers is composed of repetitive cycles of deprotection, coupling and capping. PNAs can be produced synthetically using any technique known in the art. See, e.g., U.S. Pat. Nos. 6,969,766, 7,211,668, 7,022,851, 7,125,994, 7,145,006 and 7,179,896. See also U.S. Pat. Nos. 5,539,082; 5,714,331; and 5,719,262 for the preparation of PNAs. Further teaching of PNA compounds can be found in Nielsen et al., Science, 254:1497-1500, 1991. Each of the foregoing is incorporated by reference in its entirety.

Interfering nucleic acids may also contain “locked nucleic acid” subunits (LNAs). “LNAs” are a member of a class of modifications called bridged nucleic acid (BNA). BNA is characterized by a covalent linkage that locks the conformation of the ribose ring in a C30-endo (northern) sugar pucker. For LNA, the bridge is composed of a methylene between the 2′-O and the 4′-C positions. LNA enhances backbone preorganization and base stacking to increase hybridization and thermal stability.

The structures of LNAs can be found, for example, in Wengel, et al., Chemical Communications (1998) 455; Tetrahedron (1998) 54:3607, and Accounts of Chem. Research (1999) 32:301); Obika, et al., Tetrahedron Letters (1997) 38:8735; (1998) 39:5401, and Bioorganic Medicinal Chemistry (2008) 16:9230. Compounds provided herein may incorporate one or more LNAs; in some cases, the compounds may be entirely composed of LNAs. Methods for the synthesis of individual LNA nucleoside subunits and their incorporation into oligonucleotides are described, for example, in U.S. Pat. Nos. 7,572,582, 7,569,575, 7,084,125, 7,060,809, 7,053,207, 7,034,133, 6,794,499, and 6,670,461, each of which is incorporated by reference in its entirety. Typical intersubunit linkers include phosphodiester and phosphorothioate moieties; alternatively, non-phosphorous containing linkers may be employed. One embodiment is an LNA containing compound where each LNA subunit is separated by a DNA subunit. Certain compounds are composed of alternating LNA and DNA subunits where the intersubunit linker is phosphorothioate.

“Phosphorothioates” (or S-oligos) are a variant of normal DNA in which one of the nonbridging oxygens is replaced by a sulfur. The sulfurization of the internucleotide bond reduces the action of endo- and exonucleases including 5′ to 3′ and 3′ to 5′ DNA POL 1 exonuclease, nucleases S1 and P1, RNases, serum nucleases and snake venom phosphodiesterase. Phosphorothioates are made by two principal routes: by the action of a solution of elemental sulfur in carbon disulfide on a hydrogen phosphonate, or by the method of sulfurizing phosphite triesters with either tetraethylthiuram disulfide (TETD) or 3H-1, 2-bensodithiol-3-one 1, 1-dioxide (BDTD) (see, e.g., Iyer et al., J. Org. Chem. 55, 4693-4699, 1990). The latter methods avoid the problem of elemental sulfur's insolubility in most organic solvents and the toxicity of carbon disulfide. The TETD and BDTD methods also yield higher purity phosphorothioates.

“2′O-Me oligonucleotides” molecules carry a methyl group at the 2′-OH residue of the ribose molecule. 2′-O-Me-RNAs show the same (or similar) behavior as DNA, but are protected against nuclease degradation. 2′-O-Me-RNAs can also be combined with phosphothioate oligonucleotides (PTOs) for further stabilization. 2′O-Me oligonucleotides (phosphodiester or phosphothioate) can be synthesized according to routine techniques in the art (see, e.g., Yoo et al., Nucleic Acids Res. 32:2008-16, 2004).

The interfering nucleic acids described herein may be contacted with a cell or administered to an organism (e.g., a human). Alternatively, constructs and/or vectors encoding the interfering RNA molecules may be contacted with or introduced into a cell or organism. In certain embodiments, a viral, retroviral or lentiviral vector is used. In some embodiments, the vector has a tropism for cardiac tissue. In some embodiments the vector is an adeno-associated virus.

Typically at least 17, 18, 19, 20, 21, 22 or 23 nucleotides of the complement of the target mRNA sequence are sufficient to mediate inhibition of a target transcript. Perfect complementarity is not necessary. In some embodiments, the interfering nucleic acids contains a 1, 2 or 3 nucleotide mismatch with the target sequence. The interfering nucleic acid molecule may have a 2 nucleotide 3′ overhang. If the interfering nucleic acid molecule is expressed in a cell from a construct, for example from a hairpin molecule or from an inverted repeat of the desired sequence, then the endogenous cellular machinery will create the overhangs. shRNA molecules can contain hairpins derived from microRNA molecules. For example, an RNAi vector can be constructed by cloning the interfering RNA sequence into a pCAG-miR30 construct containing the hairpin from the miR30 miRNA. RNA interference molecules may include DNA residues, as well as RNA residues.

In some embodiments, the interfering nucleic acid molecule is a siRNA molecule. Such siRNA molecules should include a region of sufficient homology to the target region, and be of sufficient length in terms of nucleotides, such that the siRNA molecule down-regulate target RNA. The term “ribonucleotide” or “nucleotide” can, in the case of a modified RNA or nucleotide surrogate, also refer to a modified nucleotide, or surrogate replacement moiety at one or more positions. It is not necessary that there be perfect complementarity between the siRNA molecule and the target, but the correspondence must be sufficient to enable the siRNA molecule to direct sequence-specific silencing, such as by RNAi cleavage of the target RNA. In some embodiments, the sense strand need only be sufficiently complementary with the antisense strand to maintain the overall double-strand character of the molecule.

In addition, an siRNA molecule may be modified or include nucleoside surrogates. Single stranded regions of an siRNA molecule may be modified or include nucleoside surrogates, e.g., the unpaired region or regions of a hairpin structure, e.g., a region which links two complementary regions, can have modifications or nucleoside surrogates. Modification to stabilize one or more 3′- or 5′-terminus of an siRNA molecule, e.g., against exonucleases, or to favor the antisense siRNA agent to enter into RISC are also useful. Modifications can include C3 (or C6, C7, C12) amino linkers, thiol linkers, carboxyl linkers, non-nucleotidic spacers (C3, C6, C9, C12, abasic, triethylene glycol, hexaethylene glycol), special biotin or fluorescein reagents that come as phosphoramidites and that have another DMT-protected hydroxyl group, allowing multiple couplings during RNA synthesis.

Each strand of an siRNA molecule can be equal to or less than 35, 30, 25, 24, 23, 22, 21, or 20 nucleotides in length. In some embodiments, the strand is at least 19 nucleotides in length. For example, each strand can be between 21 and 25 nucleotides in length. In some embodiments, siRNA agents have a duplex region of 17, 18, 19, 29, 21, 22, 23, 24, or 25 nucleotide pairs, and one or more overhangs, such as one or two 3′ overhangs, of 2-3 nucleotides.

A “small hairpin RNA” or “short hairpin RNA” or “shRNA” includes a short RNA sequence that makes a tight hairpin turn that can be used to silence gene expression via RNA interference. The shRNAs provided herein may be chemically synthesized or transcribed from a transcriptional cassette in a DNA plasmid. The shRNA hairpin structure is cleaved by the cellular machinery into siRNA, which is then bound to the RNA-induced silencing complex (RISC).

In some embodiments, shRNAs are about 15-60, 15-50, or 15-40 (duplex) nucleotides in length, about 15-30, 15-25, or 19-25 (duplex) nucleotides in length, or are about 20-24, 21-22, or 21-23 (duplex) nucleotides in length (e.g., each complementary sequence of the double-stranded shRNA is 15-60, 15-50, 15-40, 15-30, 15-25, or 19-25 nucleotides in length, or about 20-24, 21-22, or 21-23 nucleotides in length, and the double-stranded shRNA is about 15-60, 15-50, 15-40, 15-30, 15-25, or 19-25 base pairs in length, or about 18-22, 19-20, or 19-21 base pairs in length). shRNA duplexes may comprise 3′ overhangs of about 1 to about 4 nucleotides or about 2 to about 3 nucleotides on the antisense strand and/or 5′-phosphate termini on the sense strand. In some embodiments, the shRNA comprises a sense strand and/or antisense strand sequence of from about 15 to about 60 nucleotides in length (e.g., about 15-60, 15-55, 15-50, 15-45, 15-40, 15-35, 15-30, or 15-25 nucleotides in length), or from about 19 to about 40 nucleotides in length (e.g., about 19-40, 19-35, 19-30, or 19-25 nucleotides in length), or from about 19 to about 23 nucleotides in length (e.g., 19, 20, 21, 22, or 23 nucleotides in length).

Non-limiting examples of shRNA include a double-stranded polynucleotide molecule assembled from a single-stranded molecule, where the sense and antisense regions are linked by a nucleic acid-based or non-nucleic acid-based linker; and a double-stranded polynucleotide molecule with a hairpin secondary structure having self-complementary sense and antisense regions. In some embodiments, the sense and antisense strands of the shRNA are linked by a loop structure comprising from about 1 to about 25 nucleotides, from about 2 to about 20 nucleotides, from about 4 to about 15 nucleotides, from about 5 to about 12 nucleotides, or 1, 2, 3, 4, 5, 6, 7, 8, 9, 10, 11, 12, 13, 14, 15, 16, 17, 18, 19, 20, 21, 22, 23, 24, 25, or more nucleotides.

Additional embodiments related to the shRNAs, as well as methods of designing and synthesizing such shRNAs, are described in U.S. patent application publication number 2011/0071208, the disclosure of which is herein incorporated by reference in its entirety for all purposes.

In some embodiments, provided herein are micro RNAs (miRNAs). miRNAs represent a large group of small RNAs produced naturally in organisms, some of which regulate the expression of target genes. miRNAs are formed from an approximately 70 nucleotide single-stranded hairpin precursor transcript by Dicer. miRNAs are not translated into proteins, but instead bind to specific messenger RNAs, thereby blocking translation. In some instances, miRNAs base-pair imprecisely with their targets to inhibit translation.

In some embodiments, antisense oligonucleotide compounds are provided herein. In certain embodiments, the degree of complementarity between the target sequence and antisense targeting sequence is sufficient to form a stable duplex. The region of complementarity of the antisense oligonucleotides with the target RNA sequence may be as short as 8-11 bases, but can be 12-15 bases or more, e.g., 10-40 bases, 12-30 bases, 12-25 bases, 15-25 bases, 12-20 bases, or 15-20 bases, including all integers in between these ranges. An antisense oligonucleotide of about 14-15 bases is generally long enough to have a unique complementary sequence.

In certain embodiments, antisense oligonucleotides may be 100% complementary to the target sequence, or may include mismatches, e.g., to improve selective targeting of allele containing the disease-associated mutation, as long as a heteroduplex formed between the oligonucleotide and target sequence is sufficiently stable to withstand the action of cellular nucleases and other modes of degradation which may occur in vivo. Hence, certain oligonucleotides may have about or at least about 70% sequence complementarity, e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence complementarity, between the oligonucleotide and the target sequence. Oligonucleotide backbones that are less susceptible to cleavage by nucleases are discussed herein.

Mismatches, if present, are typically less destabilizing toward the end regions of the hybrid duplex than in the middle. The number of mismatches allowed will depend on the length of the oligonucleotide, the percentage of G:C base pairs in the duplex, and the position of the mismatch(es) in the duplex, according to well understood principles of duplex stability. Interfering nucleic acid molecules can be prepared, for example, by chemical synthesis, in vitro transcription, or digestion of long dsRNA by Rnase III or Dicer. These can be introduced into cells by transfection, electroporation, or other methods known in the art. See Hannon, G J, 2002, RNA Interference, Nature 418: 244-251; Bernstein E et al., 2002, The rest is silence. RNA 7: 1509-1521; Hutvagner G et al., RNAi: Nature abhors a double-strand. Curr. Opin. Genetics & Development 12: 225-232; Brummelkamp, 2002, A system for stable expression of short interfering RNAs in mammalian cells. Science 296: 550-553; Lee N S, Dohjima T, Bauer G, Li H, Li M-J, Ehsani A, Salvaterra P, and Rossi J. (2002). Expression of small interfering RNAs targeted against HIV-1 rev transcripts in human cells. Nature Biotechnol. 20:500-505; Miyagishi M, and Taira K. (2002). U6-promoter-driven siRNAs with four uridine 3′ overhangs efficiently suppress targeted gene expression in mammalian cells. Nature Biotechnol. 20:497-500; Paddison P J, Caudy A A, Bernstein E, Hannon G J, and Conklin D S. (2002). Short hairpin RNAs (shRNAs) induce sequence-specific silencing in mammalian cells. Genes & Dev. 16:948-958; Paul C P, Good P D, Winer I, and Engelke D R. (2002). Effective expression of small interfering RNA in human cells. Nature Biotechnol. 20:505-508; Sui G, Soohoo C, Affar E-B, Gay F, Shi Y, Forrester W C, and Shi Y. (2002). A DNA vector-based RNAi technology to suppress gene expression in mammalian cells. Proc. Natl. Acad. Sci. USA 99(6):5515-5520; Yu J-Y, DeRuiter S L, and Turner D L. (2002). RNA interference by expression of short-interfering RNAs and hairpin RNAs in mammalian cells. Proc. Natl. Acad. Sci. USA 99(9):6047-6052.

In the present methods, an interfering nucleic acid molecule or an interfering nucleic acid encoding polynucleotide can be administered to the subject, for example, as naked nucleic acid, in combination with a delivery reagent, and/or as a nucleic acid comprising sequences that express an interfering nucleic acid molecule. In some embodiments the nucleic acid comprising sequences that express the interfering nucleic acid molecules are delivered within vectors, e.g. plasmid, viral and bacterial vectors. Any nucleic acid delivery method known in the art can be used in the methods described herein. Suitable delivery reagents include, but are not limited to, e.g., the Mirus Transit TKO lipophilic reagent; lipofectin; lipofectamine; cellfectin; polycations (e.g., polylysine), atelocollagen, nanoplexes and liposomes. The use of atelocollagen as a delivery vehicle for nucleic acid molecules is described in Minakuchi et al. Nucleic Acids Res., 32(13):e109 (2004); Hanai et al. Ann NY Acad Sci., 1082:9-17 (2006); and Kawata et al. Mol Cancer Ther., 7(9):2904-12 (2008); each of which is incorporated herein in their entirety. Exemplary interfering nucleic acid delivery systems are provided in U.S. Pat. Nos. 8,283,461, 8,313,772, 8,501,930. 8,426,554, 8,268,798 and 8,324,366, each of which is hereby incorporated by reference in its entirety.

In some embodiments of the methods described herein, liposomes are used to deliver an inhibitory oligonucleotide to a subject. Liposomes suitable for use in the methods described herein can be formed from standard vesicle-forming lipids, which generally include neutral or negatively charged phospholipids and a sterol, such as cholesterol. The selection of lipids is generally guided by consideration of factors such as the desired liposome size and half-life of the liposomes in the blood stream. A variety of methods are known for preparing liposomes, for example, as described in Szoka et al. (1980), Ann. Rev. Biophys. Bioeng. 9:467; and U.S. Pat. Nos. 4,235,871, 4,501,728, 4,837,028, and 5,019,369, the entire disclosures of which are herein incorporated by reference.

The liposomes for use in the present methods can also be modified so as to avoid clearance by the mononuclear macrophage system (“MMS”) and reticuloendothelial system (“RES”). Such modified liposomes have opsonization-inhibition moieties on the surface or incorporated into the liposome structure.

Opsonization-inhibiting moieties for use in preparing the liposomes described herein are typically large hydrophilic polymers that are bound to the liposome membrane. As used herein, an opsonization inhibiting moiety is “bound” to a liposome membrane when it is chemically or physically attached to the membrane, e.g., by the intercalation of a lipid-soluble anchor into the membrane itself, or by binding directly to active groups of membrane lipids. These opsonization-inhibiting hydrophilic polymers form a protective surface layer that significantly decreases the uptake of the liposomes by the MMS and RES; e.g., as described in U.S. Pat. No. 4,920,016, the entire disclosure of which is herein incorporated by reference.

In some embodiments, opsonization inhibiting moieties suitable for modifying liposomes are water-soluble polymers with a number-average molecular weight from about 500 to about 40,000 daltons, or from about 2,000 to about 20,000 daltons. Such polymers include polyethylene glycol (PEG) or polypropylene glycol (PPG) derivatives; e.g., methoxy PEG or PPG, and PEG or PPG stearate; synthetic polymers such as polyacrylamide or poly N-vinyl pyrrolidone; linear, branched, or dendrimeric polyamidoamines; polyacrylic acids; polyalcohols, e.g., polyvinylalcohol and polyxylitol to which carboxylic or amino groups are chemically linked, as well as gangliosides, such as ganglioside GM1. Copolymers of PEG, methoxy PEG, or methoxy PPG, or derivatives thereof, are also suitable. In addition, the opsonization inhibiting polymer can be a block copolymer of PEG and either a polyamino acid, polysaccharide, polyamidoamine, polyethyleneamine, or polynucleotide. The opsonization inhibiting polymers can also be natural polysaccharides containing amino acids or carboxylic acids, e.g., galacturonic acid, glucuronic acid, mannuronic acid, hyaluronic acid, pectic acid, neuraminic acid, alginic acid, carrageenan; aminated polysaccharides or oligosaccharides (linear or branched); or carboxylated polysaccharides or oligosaccharides, e.g., reacted with derivatives of carbonic acids with resultant linking of carboxylic groups. In some embodiments, the opsonization-inhibiting moiety is a PEG, PPG, or derivatives thereof. Liposomes modified with PEG or PEG-derivatives are sometimes called “PEGylated liposomes.”

TABLE 1
Exemplary mRNAs

NM_001146156.2 Homo sapiens glycogen synthase kinase 3 beta (GSK3B),
transcript variant 2, mRNA (SEQ ID NO: 1)
ACACAGAGCAGCTCCTGACCCGGGCGAATGCGGGCTTGTGCCGCCGCCGCCGCCGCCGCCGCCCGGGCCA
AGTGACAAAGGAAGGAAGGAAGCGAGGAGGAGCCGGCCCCGCAGCCGCTGACAGGGCTCTGGGCTGGGGC
AAAGCGCGGACACTTCCTGAGCGGGCACCGAGCAGAGCCGAGGGGCGGGAGGGCGGCCGAGCTGTTGCCG
CGGACGGGGGAGGGGGCCCCGAGGGACGGAAGCGGTTGCCGGGTTCCCATGTCCCCGGCGAATGGGGAAC
AGTCGAGGAGCCGCTGCCTGGGGTCTGAAGGGAGCTGCCTCCGCCACCGCCATGGCCGCTGGATCCAGCC
GCCGCCTGCAGCTGCTCCTGGCGCAATGAGGAGAGGAGCCGCCGCCACCGCCACCGCCCGCCTCTGACTG
ACTCGCGACTCCGCCGCCCTCTAGTTCGCCGGGCCCCTGCCGTCAGCCCGCCGGATCCCGCGGCTTGCCG
GAGCTGCAGCGTTTCCCGTCGCATCTCCGAGCCACCCCCTCCCTCCCTCTCCCTCCCTCCTACCCATCCC
CCTTTCTCTTCAAGCGTGAGACTCGTGATCCTTCCGCCGCTTCCCTTCTTCATTGACTCGGAAAAAAAAT
CCCCGAGGAAAATATAATATTCGAAGTACTCATTTTCAATCAAGTATTTGCCCCCGTTTCACGTGATACA
TATTTTTTTAGGATTTGCCCTCTCTTTTCTCTCCTCCCAGGAAAGGGAGGGGAAAGAATTGTATTTTTTC
CCAAGTCCTAAATCATCTATATGTTAAATATCCGTGCCGATCTGTCTTGAAGGAGAAATATATCGCTTGT
TTTGTTTTTTATAGTATACAAAAGGAGTGAAAAGCCAAGAGGACGAAGTCTTTTTCTTTTTCTTCTGTGG
GAGAACTTAATGCTGCATTTATCGTTAACCTAACACCCCAACATAAAGACAAAAGGAAGAAAAGGAGGAA
GGAAGGAAAAGGTGATTCGCGAAGAGAGTGATCATGTCAGGGCGGCCCAGAACCACCTCCTTTGCGGAGA
GCTGCAAGCCGGTGCAGCAGCCTTCAGCTTTTGGCAGCATGAAAGTTAGCAGAGACAAGGACGGCAGCAA
GGTGACAACAGTGGTGGCAACTCCTGGGCAGGGTCCAGACAGGCCACAAGAAGTCAGCTATACAGACACT
AAAGTGATTGGAAATGGATCATTTGGTGTGGTATATCAAGCCAAACTTTGTGATTCAGGAGAACTGGTCG
CCATCAAGAAAGTATTGCAGGACAAGAGATTTAAGAATCGAGAGCTCCAGATCATGAGAAAGCTAGATCA
CTGTAACATAGTCCGATTGCGTTATTTCTTCTACTCCAGTGGTGAGAAGAAAGATGAGGTCTATCTTAAT
CTGGTGCTGGACTATGTTCCGGAAACAGTATACAGAGTTGCCAGACACTATAGTCGAGCCAAACAGACGC
TCCCTGTGATTTATGTCAAGTIGTATATGTATCAGCTGTTCCGAAGTTTAGCCTATATCCATTCCTTTGG
AATCTGCCATCGGGATATTAAACCGCAGAACCTCTTGTTGGATCCTGATACTGCTGTATTAAAACTCTGT
GACTTTGGAAGTGCAAAGCAGCTGGTCCGAGGAGAACCCAATGTTTCGTATATCTGTTCTCGGTACTATA
GGGCACCAGAGTTGATCTTTGGAGCCACTGATTATACCTCTAGTATAGATGTATGGTCTGCIGGCTGTGT
GTTGGCTGAGCTGTTACTAGGACAACCAATATTTCCAGGGGATAGTGGTGTGGATCAGTTGGTAGAAATA
ATCAAGGTCCTGGGAACTCCAACAAGGGAGCAAATCAGAGAAATGAACCCAAACTACACAGAATTTAAAT
TCCCTCAAATTAAGGCACATCCTTGGACTAAGGTCTTCCGACCCCGAACTCCACCGGAGGCAATTGCACT
GTGTAGCCGTCTGCTGGAGTATACACCAACTGCCCGACTAACACCACTGGAAGCTTGTGCACATTCATTT
TTTGATGAATTACGGGACCCAAATGTCAAACTACCAAATGGGCGAGACACACCTGCACTCTTCAACTTCA
CCACTCAAGAACTGTCAAGTAATCCACCTCTGGCTACCATCCTTATTCCTCCTCATGCTCGGATTCAAGC
AGCTGCTTCAACCCCCACAAATGCCACAGCAGCGTCAGATGCTAATACTGGAGACCGTGGACAGACCAAT
AATGCTGCTTCTGCATCAGCTTCCAACTCCACCTGAACAGTCCCGAGCAGCCAGCTGCACAGGAAAAACC
ACCAGTTACTTGAGTGTCACTCAGCAACACTGGTCACGTTTGGAAAGAATATTAAAAAGAGAAAAAAATC
CTGTTCATTTTAGTGTTCAATTTTTTTATTATTATTGTTGTTCTTATTTAACCTTGTAAAATATCTATAA
GAAAGCGGAGCACTAGAACATACAATCTCTCTCCCACGACAATCTTTTTTTATTAAAAGTCTGCTGTTGT
ATACTTTAAAAACAGGACTCCTGCCTCATGCCCCTTCCACAAAAGAAGAAAACCTTTTTCTGTGCTGATG
GGTTTTTTTGAACTTTGTTTTCTTTTAAAGTCTAGTGTGAGACTTTGGTATAGTGCACAGCTTGAAATTG
GTTGGGAGCTTAGCAGGTATAACTCAACGGGGACTTAAATGTCACTTGTAAAATTAATCCATATCTTCGG
GTATTTATAGACTTGCCTTTGGCATGTTGGTGGCAGGTGTGGCAGACAAAGAAATGTGTATCATTCGTAA
CCCAGGGAGGTCAATAAAGTTTGGAACTCTACAGGGAAGATTCTTAGTAGATTTGTTAAGGTTTTGTTTT
GCTCTCAGTTAGTGCTAGTGATGTAGAGGCTTGTACAGGAGGCTGCCAGAGGGGAAGCAGCAAGCAAGAC
TCAGGCACACATGCTCTACAGGTGGCTCTTTGTTTGCCTGACCAAAGTTCTTTGCAAATCTTAGCACAGT
TTCAAACTAGTGACCTGGGAGGAGATGGAAGGGGTGTTGAGCAGGCTGAGCTAGCTGCTGAGGTCAAAGG
CTGATGAGCCCAGAGGAAGGGGACAGGTCAGGGATACATCTCACCACTGTGAATAAGTTIGTCCAGATTT
TTTTCTAAAGTTACTTCCCTTGGAAAGATACACTTGAGAGGACATTGTAGTTAAATAATGTGAACTGTAA
CAGTCATCTACTGGTTTATTTTTCATATTTTTTAATTGAAAATTGAGCTTGCAGAAATAGCCACATTCTA
CACATAGTTCTAATTTTAAATCCAAATCTAGAATCTGTATTTAATTTGTTTTTTAACCTCATGCTTTTTA
CATTTATTTATTGATGCATGTCAGATGGTAGAAATATTAAAAACTACACATCAGAATGATACAGTCACTT
ATACCTGCTGACTTTATAGGAAAGCTGATGATATAAATGTGTGTATATATGTTATATATACATATATTCA
ATACTGCCTTTTTTTTTGTCTACAGTATCAAAATTGACTGGTTGAAGCATGAGAAGAATGTTTCCCCCAC
ACCCAGTTAAGAGTTTTTGTGTCTGTTTTCTTTGTGTATCAGTGAACGATGTTAAGAATCAGTCTCTCTT
ATTTTCATGACTAGTTTGTTTTATACTTTTAAGGTTGGCATCTATGTGGGCCTTATATACTCTAAAATGA
ACTTTAGTCACCTTGGTGCTTATGGGCCATTACTTGACCTATGAATCTTTAAGGCACAATCAGTIGTACT
TTACATTTAAAGATCACTTGAGTGATGGCCGCCTTTCCCTCCTACCCGCTCCTTCCCCACATGCCTTCCA
AGGTTAGCTGGTAACTGTAGGGCTGCAGAGCTGAGCCCATGGTTGTGTGTAACTTGCCCTCACCCTCCTC
ATTGCCACCTTAGGTCACTTTATGGGTCTCGTCCTCCAGAGGGTTCGGAAGTGGAGTCTGTTGGCAGCCC
TCCTGCAGGCCCTAGCACCCTGTCCTGCTCCTTAACTGTGTGTGTGACTCTCCAAGAGAGTTGTCCTGCC
TGCTGAAGTGAACCAGTACCCAGAAAGACAACTGTGAGCCATCTTGGTTTTCACTCGCTGTTTAGCTGAG
GTCTTGGGCCACAAAAGGGGTTTCACAAACCTCTGGATATATCAGAGTTTATGAGAAAGGAAACATGCTC
AGTCAAACCAAATCAAACAAATTGAATTTTATGTTTTATAAAGTGCTTCTGAAAGCTAAGATTTGAAAGA
AGTCTGAAATCAAAGTATTIGGCAGCATAACTCCTTAAAGGTAGTGGCGTTGATAGACCATTTTCAGACA
GAATTTATAAAGAATCTGAAAAGGCAGGTCTGTGATAGAGAAATGGACCTGCATTCAGATCCAACTGCCC
AGCAAGCGTTTGGATGCAGACACTGCTCTGGACGTGGTATACTCCCCAGAGTCCATAAAAATCAGTGCTT
ATTTTAGGAAACAGGTTGCCCCCCACAACTGGGGTAAAAGAAGAGAGAAAAGTCACGCTTTTCTCTCATT
TCATTGTGTGTGCATGTGTGCGTGTGTGTGTGTGTGTGTGTGTGCTGAGATGTGTGATTTTTCTTTCTCA
AGGATCATGGTGGGATCACAGAACTCTTTTATACAAGTGAGATCCAGGTCTCTGAATATCTTTTTGTATA
TAATAATAATAAAAAGCTCCTCACCAAATTCAAGCTTGTACATTATATTTTCTTTCTGTGTTTTTAAATT
TAAGTTTTATTGTTTTGTATGTAAATATGTGGACCCAGGAACTGTTATTAATGAGCAAAAAGTTACTGTT
CAGGGCAGTGATTCTGTTTAATAATCAGACAAAATGTAGACGAGCTTTTTAAAGCCATATAGTTTTAACT
CTGTACAGTAGGTACCGGCCTGTATTATTGTAACAATAACTCTAGCAATGTATAGTGTATCTATATAGTT
TGGAGTGCCTTCGCTTCCATGTGTTTTTTTTTTTAATTTGTTCTTTTTTAAATTTTAATTGGTTTCCTTT
ATCCATGTCTCCCTGTCCACCCCCTTTCCCTTTGAAATAATAACTCACTCATAACAGTATCTTTGCCCCT
TCCACAGTTAAGTTTCAGTGATACCATACTCAGGAGTGGGAAGAGGAAATCATATTCGTAATTTCATTTC
GTTGAAGCCCTGCCTTTGTTTTGGTTCTGAATGTCTTTCCTCCTCGGTAGCAGTGAGACCGGTTTCATTT
CATACTTAGTCCATTCAGGGACTTAGTGTAGCACCAGGGAGCCCTAGAGCTGGAGGATATCGAATAGATT
AAATTTTGCTCGTCTCTTCCACAAGCCCTAACCATGGGTCTTAAAAACAGCAGATTCTGGGAGCCTTCCA
TGCTCTCTCTCTCTCCTCTTTTATCTACTTCCCTCCCAAATGAGAGAGTGACAGAGAATTGTTTTTTTAT
AAATCGAAGTTTCTTAATAGTATCAGGTTTTGATACGTCAGTGGTCTAAAATGCTATAGTGCAATTACTA
GCAGTTACTGCACGGAGTGCCACCGTGCCAATAGAGGACTGTTGTTTTAACAAGGGAACTCTTAGCCCAT
TTCCTCCCTCCCGCCATCTCTACCCTTGCTCAATGAAATATCATTTTAATTTCTTTTAAAAAAAATCAGT
TTAATTCTTACTGTGTGCCCAACACGAAGGCCTTTTTTGAAAGAAAAATAGAATGTTTTGCCTCAAAGTA
GTCCATATAAAATGTCTTGAATAGAAGAAAAAACTACCAAACCAAAGGTTACTATTTTTGAAACATCGTG
TGTTCATTCCAGCAAGGCAGAAGACTGCACCTTCTTTCCAGTGACATGCTGTGTCATTTTTTTTAAGTCC
TCTTAATTTTTAGACACATTTTTGGTTTATGTTTTAACAATGTATGCCTAACCAGTCATCTTGTCTGCAC
CAATGCAAAGGTTTCTGAGAGGAGTATTCTCTATCCCTGTGGATATGAAGACACTGGCATTTCATCTATT
TTTCCCTTTCCTTTTTAAAGGATTTAACTTTGGAATCTTCCAAAGGAAGTTTGGCCAATGCCAGATCCCC
AGGAATTTGGGGGGTTTTCTTTCTTTTCAACTGAAATTGTATCTGATTCCTACTGTTCATGTTAGTGATC
ATCTAATCACAGAGCCAAACACTTTTCTCCCCTGTGTGGAAAAGTAGGTATGCTTTACAATAAAATCTGT
CTTTTCTGGTAGAAACCTGAGCCACTGAAAATAAAAGAGACAACTAGAAGCACAGTAGAGTCCCAGACTG
AGATCTACCTTTGAGAGGCTTTGAAAGTAATCCCTGGGGTTTGGATTATTTTCACAAGGGTTATGCCGTT
TTATTCAAGTTTGTTGCTCCGTTTTGCACCTCTGCAATAAAAGCAAAATGACAACCAGTACATAAGGGGT
TAGCTTGACAAAGTAGACTTCCTTGTGTTAATTTTTAAGTTTTTTTTTCCTTAACTATATCTGTCTACAG
GCAGATACAGATAGTTGTATGAAAATCTGCTTGCCTGTAAAATTTGCATTTATAAATGTGTTGCCGATGG
ATCACTTGGGCCTGTACACATACCAATTAGCGTGACCACTTCCATCTTAAAAACAAACCTAAAAAACAAA
ATTTATTATATATATATATATATATATATAAAGGACTGTGGGTTGTATACAAACTATTGCAAACACTTGT
GCAAATCTGTCTTGATATAAAGGAAAAGCAAAATCTGTATAACATTATTACTACTTGAATGCCTCTGTGA
CTGATTTTTTTTTCATTTTAAATATAAACTTTTTTGTGAAAAGTATGCTCAATGTTTTTTTTCCCTTTCC
CCATTCCCTTGTAAATACATTTTGTTCTATGTGACTTGGTTTGGAAATAGTTAACTGGTACTGTAATTTG
CATTAAATAAAAAGTAGGTTAGCCTGGAAATGAAATTAAAATTCACAAGTGTGTGGTCTTTATTTCAGTA
CCCAACCCTCTTCCTTCACCCTACTATTTTGCCGCTGCAATATGTAGTCACATCACCATTTCCATTCCTC
TAATTAGGGAAACATTAATCTTTGTTATACAGAACAAGATATCAATACCACTTCTTGTTCTTTCCAATGA
TTTTATTCCATTGTGTAGCCCCAAGAGGTGCAGCTTCCACCTTGGAAACCTTTGGATTTGATGTAGAGGA
AGCTTTGCAGACACTGCTTAGAAAAGAAAGAAAACAACTCTGAAAGGGACAGTTTTTAAATGTGTATAAG
CTGCTGTCTTTGATTACTGTGTTCATGATTTGGTGTGGCTGTATTTTCTTTTAACTTTCATCCTATTAGT
AATGGTCTTTGGGGGTCTCTGTAAAATATATGGACACCACGAACAGTGGGGCTGTACCTCCCAGGTAACC
AACACATGTTGTGTTTGAGTCTGCTCATTTCCAATACTGGATGATGTATGTAAACATGTTATGTCTCTTA
GTGCAAAAAGAAACATCATTTTTTTAGGGCTGGCTCACTCTGTCAGGCCTAATCTAAAGGCTAGATATAA
GGTCATGTGACTGCTGCTTCAATAAAAACAAATTTATATTCGA
NM_002093.4 Homo sapiens glycogen synthase kinase 3 beta (GSK3B),
transcript variant 1, mRNA (SEQ ID NO: 2)
ACACAGAGCAGCTCCTGACCCGGGCGAATGCGGGCTTGTGCCGCCGCCGCCGCCGCCGCCGCCCGGGCCA
AGTGACAAAGGAAGGAAGGAAGCGAGGAGGAGCCGGCCCCGCAGCCGCTGACAGGGCTCTGGGCTGGGGC
AAAGCGCGGACACTTCCTGAGCGGGCACCGAGCAGAGCCGAGGGGCGGGAGGGCGGCCGAGCTGTTGCCG
CGGACGGGGGAGGGGGCCCCGAGGGACGGAAGCGGTTGCCGGGTTCCCATGTCCCCGGCGAATGGGGAAC
AGTCGAGGAGCCGCTGCCTGGGGTCTGAAGGGAGCTGCCTCCGCCACCGCCATGGCCGCTGGATCCAGCC
GCCGCCTGCAGCTGCTCCTGGCGCAATGAGGAGAGGAGCCGCCGCCACCGCCACCGCCCGCCTCTGACTG
ACTCGCGACTCCGCCGCCCTCTAGTTCGCCGGGCCCCTGCCGTCAGCCCGCCGGATCCCGCGGCTTGCCG
GAGCTGCAGCGTTTCCCGTCGCATCTCCGAGCCACCCCCTCCCTCCCTCTCCCTCCCTCCTACCCATCCC
CCTTTCTCTTCAAGCGTGAGACTCGTGATCCTTCCGCCGCTTCCCTTCTTCATTGACTCGGAAAAAAAAT
CCCCGAGGAAAATATAATATTCGAAGTACTCATTTTCAATCAAGTATTTGCCCCCGTTTCACGTGATACA
TATTTTTTTAGGATTTGCCCTCTCTTTTCTCTCCTCCCAGGAAAGGGAGGGGAAAGAATTGTATTTTTTC
CCAAGTCCTAAATCATCTATATGTTAAATATCCGTGCCGATCTGTCTTGAAGGAGAAATATATCGCTTGT
TTTGTTTTTTATAGTATACAAAAGGAGTGAAAAGCCAAGAGGACGAAGTCTTTTTCTTTTTCTTCTGTGG
GAGAACTTAATGCTGCATTTATCGTTAACCTAACACCCCAACATAAAGACAAAAGGAAGAAAAGGAGGAA
GGAAGGAAAAGGTGATTCGCGAAGAGAGTGATCATGTCAGGGCGGCCCAGAACCACCTCCTTTGCGGAGA
GCTGCAAGCCGGTGCAGCAGCCTTCAGCTTTTGGCAGCATGAAAGTTAGCAGAGACAAGGACGGCAGCAA
GGTGACAACAGTGGTGGCAACTCCTGGGCAGGGTCCAGACAGGCCACAAGAAGTCAGCTATACAGACACT
AAAGTGATTGGAAATGGATCATTTGGTGTGGTATATCAAGCCAAACTTTGTGATTCAGGAGAACTGGTCG
CCATCAAGAAAGTATTGCAGGACAAGAGATTTAAGAATCGAGAGCTCCAGATCATGAGAAAGCTAGATCA
CTGTAACATAGTCCGATTGCGTTATTTCTTCTACTCCAGTGGTGAGAAGAAAGATGAGGTCTATCTTAAT
CTGGTGCTGGACTATGTTCCGGAAACAGTATACAGAGTTGCCAGACACTATAGTCGAGCCAAACAGACGC
TCCCTGTGATTTATGTCAAGTTGTATATGTATCAGCTGTTCCGAAGTTTAGCCTATATCCATTCCTTTGG
AATCTGCCATCGGGATATTAAACCGCAGAACCTCTTGTTGGATCCTGATACTGCTGTATTAAAACTCTGT
GACTTTGGAAGTGCAAAGCAGCTGGTCCGAGGAGAACCCAATGTTTCGTATATCTGTTCTCGGTACTATA
GGGCACCAGAGTTGATCTTTGGAGCCACTGATTATACCTCTAGTATAGATGTATGGTCTGCIGGCTGTGT
GTTGGCTGAGCTGTTACTAGGACAACCAATATTTCCAGGGGATAGTGGTGTGGATCAGTTGGTAGAAATA
ATCAAGGTCCTGGGAACTCCAACAAGGGAGCAAATCAGAGAAATGAACCCAAACTACACAGAATTTAAAT
TCCCTCAAATTAAGGCACATCCTTGGACTAAGGATTCGTCAGGAACAGGACATTTCACCTCAGGAGTGCG
GGTCTTCCGACCCCGAACTCCACCGGAGGCAATTGCACTGTGTAGCCGTCTGCTGGAGTATACACCAACT
GCCCGACTAACACCACTGGAAGCTTGTGCACATTCATTTTTTGATGAATTACGGGACCCAAATGTCAAAC
TACCAAATGGGCGAGACACACCTGCACTCTTCAACTTCACCACTCAAGAACTGTCAAGTAATCCACCTCT
GGCTACCATCCTTATTCCTCCTCATGCTCGGATTCAAGCAGCTGCTTCAACCCCCACAAATGCCACAGCA
GCGTCAGATGCTAATACTGGAGACCGTGGACAGACCAATAATGCTGCTTCTGCATCAGCTTCCAACTCCA
CCTGAACAGTCCCGAGCAGCCAGCTGCACAGGAAAAACCACCAGTTACTTGAGTGTCACTCAGCAACACT
GGTCACGTTTGGAAAGAATATTAAAAAGAGAAAAAAATCCTGTTCATTTTAGTGTTCAATTTTTTTATTA
TTATTGTTGTTCTTATTTAACCTTGTAAAATATCTATAAATACAAACCAATTTCATTGTATTCTCACTTT
GAGGGAGATCCAGGGGGTGGGAGGGGTTGTGGGGAGGGGGAAAGCGGAGCACTAGAACATACAATCTCTC
TCCCACGACAATCTTTTTTTATTAAAAGTCTGCTGTTGTATACTTTAAAAACAGGACTCCTGCCTCATGC
CCCTTCCACAAAAGAAGAAAACCTTTTTCTGTGCTGATGGGTTTTTTTGAACTTTGTTTTCTTTTAAAGT
CTAGTGTGAGACTTTGGTATAGTGCACAGCTTGAAATTGGTTGGGAGCTTAGCAGGTATAACTCAACGGG
GACTTAAATGTCACTTGTAAAATTAATCCATATCTTCGGGTATTTATAGACTTGCCTTTGGCATGTTGGT
GGCAGGTGTGGCAGACAAAGAAATGTGTATCATTCGTAACCCAGGGAGGTCAATAAAGTTTGGAACTCTA
CAGGGAAGATTCTTAGTAGATTTGTTAAGGTTTTGTTTTGCTCTCAGTTAGTGCTAGTGATGTAGAGGCT
TGTACAGGAGGCTGCCAGAGGGGAAGCAGCAAGCAAGACTCAGGCACACATGCTCTACAGGTGGCTCTTT
GTTTGCCTGACCAAAGTTCTTTGCAAATCTTAGCACAGTTTCAAACTAGTGACCTGGGAGGAGATGGAAG
GGGTGTTGAGCAGGCTGAGCTAGCTGCTGAGGTCAAAGGCTGATGAGCCCAGAGGAAGGGGACAGGTCAG
GGATACATCTCACCACTGTGAATAAGTTTGTCCAGATTTTTTTCTAAAGTTACTTCCCTTGGAAAGATAC
ACTTGAGAGGACATTGTAGTTAAATAATGTGAACTGTAACAGTCATCTACTGGTTTATTTTTCATATTTT
TTAATTGAAAATTGAGCTTGCAGAAATAGCCACATTCTACACATAGTTCTAATTTTAAATCCAAATCTAG
AATCTGTATTTAATTTGTTTTTTAACCTCATGCTTTTTACATTTATTTATTGATGCATGTCAGATGGTAG
AAATATTAAAAACTACACATCAGAATGATACAGTCACTTATACCTGCTGACTTTATAGGAAAGCTGATGA
TATAAATGTGTGTATATATGTTATATATACATATATTCAATACTGCCTTTTTTTTTGTCTACAGTATCAA
AATTGACTGGTTGAAGCATGAGAAGAATGTTTCCCCCACACCCAGTTAAGAGTTTTTGTGTCTGTTTTCT
TTGTGTATCAGTGAACGATGTTAAGAATCAGTCTCTCTTTTTGAAGAAAAAGCAATATTCCTTGGAAAGC
AAGGAGAATTGAAGGACTATGTTTGCCGTGAGGAAATAGATTTTCATGACTAGTTTGTTTTATACTTTTA
AGGTTGGCATCTATGTGGGCCTTATATACTCTAAAATGAACTTTAGTCACCTTGGTGCTTATGGGCCATT
CCTTTCCCTCCTACCCGCTCCTTCCCCACATGCCTTCCAAGGTTAGCTGGTAACTGTAGGGCTGCAGAGC
TGAGCCCATGGTTGTGTGTAACTTGCCCTCACCCTCCTCATTGCCACCTTAGGTCACTTTATGGGTCTCG
TCCTCCAGAGGGTTCGGAAGTGGAGTCTGTTGGCAGCCCTCCTGCAGGCCCTAGCACCCTGTCCTGCTCC
TTAACTGTGTGTGTGACTCTCCAAGAGAGTTGTCCTGCCTGCTGAAGTGAACCAGTACCCAGAAAGACAA
CTGTGAGCCATCTTGGTTTTCACTCGCTGTTTAGCTGAGGTCTTGGGCCACAAAAGGGGTTTCACAAACC
TCTGGATATATCAGAGTTTATGAGAAAGGAAACATGCTCAGTCAAACCAAATCAAACAAATTGAATTTTA
TGTTTTATAAAGTGCTTCTGAAAGCTAAGATTTGAAAGAAGTCTGAAATCAAAGTATTTGGCAGCATAAC
TCCTTAAAGGTAGTGGCGTTGATAGACCATTTTCAGACAGAATTTATAAAGAATCTGAAAAGGCAGGTCT
GTGATAGAGAAATGGACCTGCATTCAGATCCAACTGCCCAGCAAGCGTTTGGATGCAGACACTGCTCTGG
ACGTGGTATACTCCCCAGAGTCCATAAAAATCAGTGCTTATTTTAGGAAACAGGTTGCCCCCCACAACTG
GTGTGTGTGTGTGCTGAGATGTGTGATTTTTCTTTCTCAAGGATCATGGTGGGATCACAGAACTCTTTTA
TACAAGTGAGATCCAGGTCTCTGAATATCTTTTTGTATATAATAATAATAAAAAGCTCCTCACCAAATTC
AAGCTTGTACATTATATTTTCTTTCTGTGTTTTTAAATTTAAGTTTTATTGTTTTGTATGTAAATATGTG
GACCCAGGAACTGTTATTAATGAGCAAAAAGTTACTGTTCAGGGCAGTGATTCTGTTTAATAATCAGACA
AAATGTAGACGAGCTTTTTAAAGCCATATAGTTTTAACTCTGTACAGTAGGTACCGGCCTGTATTATTGT
AACAATAACTCTAGCAATGTATAGTGTATCTATATAGTTTGGAGTGCCTTCGCTTCCATGTGTTTTTTTT
TTTAATTTGTTCTTTTTTAAATTTTAATTGGTTTCCTTTATCCATGTCTCCCTGTCCACCCCCTTTCCCT
TTGAAATAATAACTCACTCATAACAGTATCTTTGCCCCTTCCACAGTTAAGTTTCAGTGATACCATACTC
AGGAGTGGGAAGAGGAAATCATATTCGTAATTTCATTTCGTTGAAGCCCTGCCTTTGTTTTGGTTCTGAA
TGTCTTTCCTCCTCGGTAGCAGTGAGACCGGTTTCATTTCATACTTAGTCCATTCAGGGACTTAGTGTAG
CACCAGGGAGCCCTAGAGCTGGAGGATATCGAATAGATTAAATTTTGCTCGTCTCTTCCACAAGCCCTAA
CCATGGGTCTTAAAAACAGCAGATTCTGGGAGCCTTCCATGCTCTCTCTCTCTCCTCTTTTATCTACTTC
CCTCCCAAATGAGAGAGTGACAGAGAATTGTTTTTTTATAAATCGAAGTTTCTTAATAGTATCAGGTTTT
GATACGTCAGTGGTCTAAAATGCTATAGTGCAATTACTAGCAGTTACTGCACGGAGTGCCACCGTGCCAA
TAGAGGACTGTTGTTTTAACAAGGGAACTCTTAGCCCATTTCCTCCCTCCCGCCATCTCTACCCTTGCTC
AATGAAATATCATTTTAATTTCTTTTAAAAAAAATCAGTTTAATTCTTACTGTGTGCCCAACACGAAGGC
CTTTTTTGAAAGAAAAATAGAATGTTTTGCCTCAAAGTAGTCCATATAAAATGTCTTGAATAGAAGAAAA
AACTACCAAACCAAAGGTTACTATTTTTGAAACATCGTGTGTTCATTCCAGCAAGGCAGAAGACTGCACC
TTCTTTCCAGTGACATGCTGTGTCATTTTTTTTAAGTCCTCTTAATTTTTAGACACATTTTTGGTTTATG
TTTTAACAATGTATGCCTAACCAGTCATCTTGTCTGCACCAATGCAAAGGTTTCTGAGAGGAGTATTCTC
TATCCCTGTGGATATGAAGACACTGGCATTTCATCTATTTTTCCCTTTCCTTTTTAAAGGATTTAACTTT
GGAATCTTCCAAAGGAAGTTTGGCCAATGCCAGATCCCCAGGAATTTGGGGGGTTTTCTTTCTTTTCAAC
TGAAATTGTATCTGATTCCTACTGTTCATGTTAGTGATCATCTAATCACAGAGCCAAACACTTTTCTCCC
CTGTGTGGAAAAGTAGGTATGCTTTACAATAAAATCTGTCTTTTCTGGTAGAAACCTGAGCCACTGAAAA
TAAAAGAGACAACTAGAAGCACAGTAGAGTCCCAGACTGAGATCTACCTTTGAGAGGCTTTGAAAGTAAT
CCCTGGGGTTTGGATTATTTTCACAAGGGTTATGCCGTTTTATTCAAGTTTGTTGCTCCGTTTTGCACCT
CTGCAATAAAAGCAAAATGACAACCAGTACATAAGGGGTTAGCTTGACAAAGTAGACTTCCTTGTGTTAA
TTTTTAAGTTTTTTTTTCCTTAACTATATCTGTCTACAGGCAGATACAGATAGTTGTATGAAAATCTGCT
TGCCTGTAAAATTTGCATTTATAAATGTGTTGCCGATGGATCACTTGGGCCTGTACACATACCAATTAGC
GTGACCACTTCCATCTTAAAAACAAACCTAAAAAACAAAATTTATTATATATATATATATATATATATAA
AGGACTGTGGGTTGTATACAAACTATTGCAAACACTTGTGCAAATCTGTCTTGATATAAAGGAAAAGCAA
AATCTGTATAACATTATTACTACTTGAATGCCTCTGTGACTGATTTTTTTTTCATTTTAAATATAAACTT
TTTTGTGAAAAGTATGCTCAATGTTTTTTTTCCCTTTCCCCATTCCCTTGTAAATACATTTTGTTCTATG
TGACTTGGTTTGGAAATAGTTAACTGGTACTGTAATTTGCATTAAATAAAAAGTAGGTTAGCCTGGAAAT
GAAATTAAAATTCACAAGTGTGTGGTCTTTATTICAGTACCCAACCCTCTTCCTTCACCCTACTATTTTG
CCGCTGCAATATGTAGTCACATCACCATTTCCATTCCTCTAATTAGGGAAACATTAATCTTTGTTATACA
GAACAAGATATCAATACCACTTCTTGTTCTTTCCAATGATTTTATTCCATTGTGTAGCCCCAAGAGGTGC
AGCTTCCACCTTGGAAACCTTTGGATTTGATGTAGAGGAAGCTTTGCAGACACTGCTTAGAAAAGAAAGA
AAACAACTCTGAAAGGGACAGTTTTTAAATGTGTATAAGCTGCTGTCTTTGATTACTGIGTTCATGATTT
GGTGTGGCTGTATTTTCTTTTAACTTTCATCCTATTAGTAATGGTCTTTGGGGGTCTCTGTAAAATATAT
GGACACCACGAACAGTGGGGCTGTACCTCCCAGGTAACCAACACATGTTGTGTTTGAGTCTGCTCATTTC
CAATACTGGATGATGTATGTAAACATGTTATGTCTCTTAGTGCAAAAAGAAACATCATTTTTTTAGGGCT
GGCTCACTCTGTCAGGCCTAATCTAAAGGCTAGATATAAGGTCATGTGACTGCTGCTTCAATAAAAACAA
ATTTATATTCGA
NM_001354596.2 Homo sapiens glycogen synthase kinase 3 beta (GSK3B),
transcript variant 3, mRNA (SEQ ID NO: 3)
ACACAGAGCAGCTCCTGACCCGGGCGAATGCGGGCTTGTGCCGCCGCCGCCGCCGCCGCCGCCCGGGCCA
AGTGACAAAGGAAGGAAGGAAGCGAGGAGGAGCCGGCCCCGCAGCCGCTGACAGGGCTCTGGGCTGGGGC
AAAGCGCGGACACTTCCTGAGCGGGCACCGAGCAGAGCCGAGGGGCGGGAGGGCGGCCGAGCTGTTGCCG
CGGACGGGGGAGGGGGCCCCGAGGGACGGAAGCGGTTGCCGGGTTCCCATGTCCCCGGCGAATGGGGAAC
AGTCGAGGAGCCGCTGCCTGGGGTCTGAAGGGAGCTGCCTCCGCCACCGCCATGGCCGCTGGATCCAGCC
GCCGCCTGCAGCTGCTCCTGGCGCAATGAGGAGAGGAGCCGCCGCCACCGCCACCGCCCGCCTCTGACTG
ACTCGCGACTCCGCCGCCCTCTAGTTCGCCGGGCCCCTGCCGTCAGCCCGCCGGATCCCGCGGCTTGCCG
GAGCTGCAGCGTTTCCCGTCGCATCTCCGAGCCACCCCCTCCCTCCCTCTCCCTCCCTCCTACCCATCCC
CCTTTCTCTTCAAGCGTGAGACTCGTGATCCTTCCGCCGCTTCCCTTCTTCATTGACTCGGAAAAAAAAT
CCCCGAGGAAAATATAATATTCGAAGTACTCATTTTCAATCAAGTATTTGCCCCCGTTTCACGTGATACA
TATTTTTTTAGGATTTGCCCTCTCTTTTCTCTCCTCCCAGGAAAGGGAGGGGAAAGAATTGTATTTTTTC
CCAAGTCCTAAATCATCTATATGTTAAATATCCGTGCCGATCTGTCTTGAAGGAGAAATATATCGCTTGT
TTTGTTTTTTATAGTATACAAAAGGAGTGAAAAGCCAAGAGGACGAAGTCTTTTTCTTTTTCTTCTGIGG
GAGAACTTAATGCTGCATTTATCGTTAACCTAACACCCCAACATAAAGACAAAAGGAAGAAAAGGAGGAA
GGAAGGAAAAGGTGATTCGCGAAGAGAGTGATCATGTCAGGGCGGCCCAGAACCACCTCCTTTGCGGAGA
GCTGCAAGCCGGTGCAGCAGCCTTCAGCTTTTGGCAGCATGAAAGTTAGCAGAGACAAGGACGGCAGCAA
GGTGACAACAGTGGTGGCAACTCCTGGGCAGGGTCCAGACAGGCCACAAGAAGTCAGCTATACAGACACT
AAAGTGATTGGAAATGGATCATTTGGTGTGGTATATCAAGCCAAACTTTGTGATTCAGGAGAACTGGTCG
CCATCAAGAAAGTATTGCAGGACAAGAGATTTAAGAATCGAGAGCTCCAGATCATGAGAAAGCTAGATCA
CTGTAACATAGTCCGATTGCGTTATTTCTTCTACTCCAGTGGTGAGAAGAAAGATGAGGTCTATCITAAT
CTGGTGCTGGACTATGTTCCGGAAACAGTATACAGAGTTGCCAGACACTATAGTCGAGCCAAACAGACGC
TCCCTGTGATTTATGTCAAGTTGTATATGTATCAGCTGTTCCGAAGTTTAGCCTATATCCATTCCTTTGG
AATCTGCCATCGGGATATTAAACCGCAGAACCTCTTGTTGGATCCTGATACTGCTGTATTAAAACTCTGT
GACTTTGGAAGTGCAAAGCAGCTGGTCCGAGGAGAACCCAATGTTTCGTATATCTGTTCTCGGTACTATA
GGGCACCAGAGTTGATCTTTGGAGCCACTGATTATACCTCTAGTATAGATGTATGGTCTGCTGGCTGTGT
GTTGGCTGAGCTGTTACTAGGACAACCAATATTTCCAGGGGATAGTGGTGTGGATCAGTTGGTAGAAATA
ATCAAGGTCCTGGGAACTCCAACAAGGGAGCAAATCAGAGAAATGAACCCAAACTACACAGAATTTAAAT
TCCCTCAAATTAAGGCACATCCTTGGACTAAGGTCTTCCGACCCCGAACTCCACCGGAGGCAATTGCACT
GTGTAGCCGTCTGCTGGAGTATACACCAACTGCCCGACTAACACCACTGGAAGCTTGTGCACATTCATTT
TTTGATGAATTACGGGACCCAAATGTCAAACTACCAAATGGGCGAGACACACCTGCACTCTTCAACTTCA
CCACTCAAGATGCTAATACTGGAGACCGTGGACAGACCAATAATGCTGCTTCTGCATCAGCTTCCAACTC
CACCTGAACAGTCCCGAGCAGCCAGCTGCACAGGAAAAACCACCAGTTACTTGAGTGTCACTCAGCAACA
CTGGTCACGTTTGGAAAGAATATTAAAAAGAGAAAAAAATCCTGTTCATTTTAGTGTTCAATTTTTTTAT
TATTATTGTTGTTCTTATTTAACCTTGTAAAATATCTATAAATACAAACCAATTTCATTGTATTCTCACT
TTGAGGGAGATCCAGGGGGTGGGAGGGGTTGTGGGGAGGGGGAAAGCGGAGCACTAGAACATACAATCTC
TCTCCCACGACAATCTTTTTTTATTAAAAGTCTGCTGTTGTATACTTTAAAAACAGGACTCCTGCCTCAT
GCCCCTTCCACAAAAGAAGAAAACCTTTTTCTGTGCTGATGGGTTTTTTTGAACTTTGTTTTCTTTTAAA
GTCTAGTGTGAGACTTTGGTATAGTGCACAGCTTGAAATTGGTTGGGAGCTTAGCAGGTATAACTCAACG
GGGACTTAAATGTCACTTGTAAAATTAATCCATATCTTCGGGTATTTATAGACTTGCCTTTGGCATGTTG
GTGGCAGGTGTGGCAGACAAAGAAATGTGTATCATTCGTAACCCAGGGAGGTCAATAAAGTTTGGAACTC
TACAGGGAAGATTCTTAGTAGATTTGTTAAGGTTTTGTTTTGCTCTCAGTTAGTGCTAGTGATGTAGAGG
CTTGTACAGGAGGCTGCCAGAGGGGAAGCAGCAAGCAAGACTCAGGCACACATGCTCTACAGGTGGCTCT
TTGTTTGCCTGACCAAAGTTCTTTGCAAATCTTAGCACAGTTTCAAACTAGTGACCTGGGAGGAGATGGA
AGGGGTGTTGAGCAGGCTGAGCTAGCTGCTGAGGTCAAAGGCTGATGAGCCCAGAGGAAGGGGACAGGTC
AGGGATACATCTCACCACTGTGAATAAGTTTGTCCAGATTTTTTTCTAAAGTTACTTCCCTTGGAAAGAT
ACACTTGAGAGGACATTGTAGTTAAATAATGTGAACTGTAACAGTCATCTACTGGTTTATTTTTCATATT
TTTTAATTGAAAATTGAGCTTGCAGAAATAGCCACATTCTACACATAGTTCTAATTTTAAATCCAAATCT
AGAATCTGTATTTAATTTGTTTTTTAACCTCATGCTTTTTACATTTATTTATTGATGCATGTCAGATGGT
AGAAATATTAAAAACTACACATCAGAATGATACAGTCACTTATACCTGCTGACTTTATAGGAAAGCTGAT
GATATAAATGTGTGTATATATGTTATATATACATATATTCAATACTGCCTTTTTTTTTGTCTACAGTATC
AAAATTGACTGGTTGAAGCATGAGAAGAATGTTTCCCCCACACCCAGTTAAGAGTTTTTGTGTCTGTTTT
CTTTGTGTATCAGTGAACGATGTTAAGAATCAGTCTCTCTTTTTGAAGAAAAAGCAATATTCCTTGGAAA
TAAGGTTGGCATCTATGTGGGCCTTATATACTCTAAAATGAACTTTAGTCACCTTGGTGCTTATGGGCCA
TTACTTGACCTATGAATCTTTAAGGCACAATCAGTTGTACTTTACATTTAAAGATCACTTGAGTGATGGC
CGCCTTTCCCTCCTACCCGCTCCTTCCCCACATGCCTTCCAAGGTTAGCTGGTAACTGTAGGGCTGCAGA
GCTGAGCCCATGGTTGTGTGTAACTTGCCCTCACCCTCCTCATTGCCACCTTAGGTCACTTTATGGGTCT
CGTCCTCCAGAGGGTTCGGAAGTGGAGTCTGTTGGCAGCCCTCCTGCAGGCCCTAGCACCCTGTCCTGCT
CCTTAACTGTGTGTGTGACTCTCCAAGAGAGTTGTCCTGCCTGCTGAAGTGAACCAGTACCCAGAAAGAC
AACTGTGAGCCATCTTGGTTTTCACTCGCTGTTTAGCTGAGGTCTTGGGCCACAAAAGGGGTTTCACAAA
CCTCTGGATATATCAGAGTTTATGAGAAAGGAAACATGCTCAGTCAAACCAAATCAAACAAATTGAATTT
TATGTTTTATAAAGTGCTTCTGAAAGCTAAGATTTGAAAGAAGTCTGAAATCAAAGTATTTGGCAGCATA
ACTCCTTAAAGGTAGTGGCGTTGATAGACCATTTTCAGACAGAATTTATAAAGAATCTGAAAAGGCAGGT
CTGTGATAGAGAAATGGACCTGCATTCAGATCCAACTGCCCAGCAAGCGTTTGGATGCAGACACTGCTCT
GGACGTGGTATACTCCCCAGAGTCCATAAAAATCAGTGCTTATTTTAGGAAACAGGTTGCCCCCCACAAC
TGGGGTAAAAGAAGAGAGAAAAGTCACGCTTTTCTCTCATTTCATTGTGTGTGCATGTGTGCGTGTGIGT
GTGTGTGTGTGTGTGCTGAGATGTGTGATTTTTCTTTCTCAAGGATCATGGTGGGATCACAGAACTCTTT
TATACAAGTGAGATCCAGGTCTCTGAATATCTTTTTGTATATAATAATAATAAAAAGCTCCTCACCAAAT
TCAAGCTTGTACATTATATTTTCTTTCTGTGTTTTTAAATTTAAGTTTTATTGTTTTGTATGTAAATATG
TGGACCCAGGAACTGTTATTAATGAGCAAAAAGTTACTGTTCAGGGCAGTGATTCTGTTTAATAATCAGA
CAAAATGTAGACGAGCTTTTTAAAGCCATATAGTTTTAACTCTGTACAGTAGGTACCGGCCTGTATTATT
GTAACAATAACTCTAGCAATGTATAGTGTATCTATATAGTTTGGAGTGCCTTCGCTTCCATGTGTTTTTT
TTTTTAATTTGTTCTTTTTTAAATTTTAATTGGTTTCCTTTATCCATGTCTCCCTGTCCACCCCCTTTCC
CTTTGAAATAATAACTCACTCATAACAGTATCTTTGCCCCTTCCACAGTTAAGTTTCAGTGATACCATAC
TCAGGAGTGGGAAGAGGAAATCATATTCGTAATTTCATTTCGTTGAAGCCCTGCCTTTGTTTTGGTTCTG
AATGTCTTTCCTCCTCGGTAGCAGTGAGACCGGTTTCATTTCATACTTAGTCCATTCAGGGACTTAGTGT
AGCACCAGGGAGCCCTAGAGCTGGAGGATATCGAATAGATTAAATTTTGCTCGTCTCTTCCACAAGCCCT
AACCATGGGTCTTAAAAACAGCAGATTCTGGGAGCCTTCCATGCTCTCTCTCTCTCCTCTTTTATCTACT
TCCCTCCCAAATGAGAGAGTGACAGAGAATTGTTTTTTTATAAATCGAAGTTTCTTAATAGTATCAGGTT
TTGATACGTCAGTGGTCTAAAATGCTATAGTGCAATTACTAGCAGTTACTGCACGGAGTGCCACCGTGCC
AATAGAGGACTGTTGTTTTAACAAGGGAACTCTTAGCCCATTTCCTCCCTCCCGCCATCTCTACCCTTGC
TCAATGAAATATCATTTTAATTTCTTTTAAAAAAAATCAGTTTAATTCTTACTGTGTGCCCAACACGAAG
GCCTTTTTTGAAAGAAAAATAGAATGTTTTGCCTCAAAGTAGTCCATATAAAATGTCTTGAATAGAAGAA
AAAACTACCAAACCAAAGGTTACTATTTTTGAAACATCGTGTGTTCATTCCAGCAAGGCAGAAGACTGCA
CCTTCTTTCCAGTGACATGCTGTGTCATTTTTTTTAAGTCCTCTTAATTTTTAGACACATTTTTGGTTTA
TGTTTTAACAATGTATGCCTAACCAGTCATCTTGTCTGCACCAATGCAAAGGTTTCTGAGAGGAGTATTC
TCTATCCCTGTGGATATGAAGACACTGGCATTTCATCTATTTTTCCCTTTCCTTTTTAAAGGATTTAACT
TTGGAATCTTCCAAAGGAAGTTTGGCCAATGCCAGATCCCCAGGAATTTGGGGGGTTTTCTTTCTTTTCA
ACTGAAATTGTATCTGATTCCTACTGTTCATGTTAGTGATCATCTAATCACAGAGCCAAACACTTTTCTC
CCCTGTGTGGAAAAGTAGGTATGCTTTACAATAAAATCTGTCTTTTCTGGTAGAAACCTGAGCCACTGAA
AATAAAAGAGACAACTAGAAGCACAGTAGAGTCCCAGACTGAGATCTACCTTTGAGAGGCTTTGAAAGTA
ATCCCTGGGGTTTGGATTATTTTCACAAGGGTTATGCCGTTTTATTCAAGTTTGTTGCTCCGTTTTGCAC
CTCTGCAATAAAAGCAAAATGACAACCAGTACATAAGGGGTTAGCTTGACAAAGTAGACTTCCTTGTGTT
AATTTTTAAGTTTTTTTTTCCTTAACTATATCTGTCTACAGGCAGATACAGATAGTTGTATGAAAATCTG
CTTGCCTGTAAAATTTGCATTTATAAATGTGTTGCCGATGGATCACTTGGGCCTGTACACATACCAATTA
GCGTGACCACTTCCATCTTAAAAACAAACCTAAAAAACAAAATTTATTATATATATATATATATATATAT
AAAGGACTGTGGGTTGTATACAAACTATTGCAAACACTTGTGCAAATCTGTCTTGATATAAAGGAAAAGC
AAAATCTGTATAACATTATTACTACTTGAATGCCTCTGTGACTGATTTTTTTTTCATTTTAAATATAAAC
TTTTTTGTGAAAAGTATGCTCAATGTTTTTTTTCCCTTTCCCCATTCCCTTGTAAATACATTTTGTTCTA
TGTGACTTGGTTTGGAAATAGTTAACTGGTACTGTAATTTGCATTAAATAAAAAGTAGGTTAGCCTGGAA
ATGAAATTAAAATTCACAAGTGTGTGGTCTTTATTTCAGTACCCAACCCTCTTCCTTCACCCTACTATTT
TGCCGCTGCAATATGTAGTCACATCACCATTTCCATTCCTCTAATTAGGGAAACATTAATCTTTGTTATA
CAGAACAAGATATCAATACCACTTCTTGTTCTTTCCAATGATTTTATTCCATTGTGTAGCCCCAAGAGGT
GCAGCTTCCACCTTGGAAACCTTTGGATTTGATGTAGAGGAAGCTTTGCAGACACTGCTTAGAAAAGAAA
GAAAACAACTCTGAAAGGGACAGTTTTTAAATGTGTATAAGCTGCTGTCTTTGATTACTGTGTTCATGAT
TTGGTGTGGCTGTATTTTCTTTTAACTTTCATCCTATTAGTAATGGTCTTTGGGGGTCTCTGTAAAATAT
ATGGACACCACGAACAGTGGGGCTGTACCTCCCAGGTAACCAACACATGTTGTGTTTGAGTCTGCTCATT
TCCAATACTGGATGATGTATGTAAACATGTTATGTCTCTTAGTGCAAAAAGAAACATCATTTTTTTAGGG
CTGGCTCACTCTGTCAGGCCTAATCTAAAGGCTAGATATAAGGTCATGTGACTGCTGCTTCAATAAAAAC
AAATTTATATTCGA
NM_005900.3 Homo sapiens SMAD family member 1 (SMAD1), transcript
variant 1, mRNA (SEQ ID NO: 4)
AGATCAATCCAGGCTCCAGGAGAAAGCAGGCGGGCGGGCGGAGAAAGGAGAGGCCGAGCGGCTCAACCCG
GGCCGAGGCTCGGGGAGCGGAGAGTGGCGCAGCGCCCGGCCGTCCGGACCCGGGCCGCGAGACCCCGCTC
GCCCGGCCACTCGTGCTCCCACACGGACGGGCGCGCCGCCAACCCGGTGCTGACTGGGTTACTTTTTTAA
ACACTAGGAATGGTAATTTCTACTCTTCTGGACTTCAAACTAAGAAGTTAAAGAGACTTCTCTGTAAATA
AACAAATCTCTTCTGCTGTCCTTTTGCATTTGGAGACAGCTTTATTICACCATATCCAAGGAGTATAACT
AGTGCTGTCATTATGAATGTGACAAGTTTATTTTCCTTTACAAGTCCAGCTGTGAAGAGACTTCTTGGGT
GGAAACAGGGCGATGAAGAAGAAAAATGGGCAGAGAAAGCTGTTGATGCTTTGGTGAAAAAACTGAAGAA
AAAGAAAGGTGCCATGGAGGAACTGGAAAAGGCCTTGAGCTGCCCAGGGCAACCGAGTAACTGTGTCACC
ATTCCCCGCTCTCTGGATGGCAGGCTGCAAGTCTCCCACCGGAAGGGACTGCCTCATGTCATTTACTGCC
GTGTGTGGCGCTGGCCCGATCTTCAGAGCCACCATGAACTAAAACCACTGGAATGCTGTGAGTTTCCTTT
TGGTTCCAAGCAGAAGGAGGTCTGCATCAATCCCTACCACTATAAGAGAGTAGAAAGCCCTGTACTTCCT
CCTGTGCTGGTTCCAAGACACAGCGAATATAATCCTCAGCACAGCCTCTTAGCTCAGTTCCGTAACTTAG
GACAAAATGAGCCTCACATGCCACTCAACGCCACTTTTCCAGATTCTTTCCAGCAACCCAACAGCCACCC
GTTTCCTCACTCTCCCAATAGCAGTTACCCAAACTCTCCTGGGAGCAGCAGCAGCACCTACCCTCACTCT
CCCACCAGCTCAGACCCAGGAAGCCCTTTCCAGATGCCAGCTGATACGCCCCCACCTGCTTACCTGCCTC
CTGAAGACCCCATGACCCAGGATGGCTCTCAGCCGATGGACACAAACATGATGGCGCCTCCCCTGCCCTC
AGAAATCAACAGAGGAGATGTTCAGGCGGTTGCTTATGAGGAACCAAAACACTGGTGCTCTATTGTCTAC
TATGAGCTCAACAATCGTGTGGGTGAAGCGTTCCATGCCTCCTCCACAAGTGTGTTGGTGGATGGTTTCA
CTGATCCTTCCAACAATAAGAACCGTTTCTGCCTTGGGCTGCTCTCCAATGTTAACCGGAATTCCACTAT
TGAAAACACCAGGCGGCATATTGGAAAAGGAGTTCATCTTTATTATGTTGGAGGGGAGGTGTATGCCGAA
TGCCTTAGTGACAGTAGCATCTTTGTGCAAAGTCGGAACTGCAACTACCATCATGGATTTCATCCTACTA
CTGTTTGCAAGATCCCTAGTGGGTGTAGTCTGAAAATTTTTAACAACCAAGAATTTGCTCAGTTATTGGC
ACAGTCTGTGAACCATGGATTTGAGACAGTCTATGAGCTTACAAAAATGTGTACTATACGTATGAGCTTT
GTGAAGGGCTGGGGAGCAGAATACCACCGCCAGGATGTTACTAGCACCCCCTGCTGGATTGAGATACATC
TGCACGGCCCCCTCCAGTGGCTGGATAAAGTTCTTACTCAAATGGGTTCACCTCATAATCCTATTTCATC
TGTATCTTAAATGGCCCCAGGCATCTGCCTCTGGAAAACTATTGAGCCTTGCATGTACTTGAAGGATGGA
TGAGTCAGACACGATTGAGAACTGACAAAGGAGCCTTGATAATACTTGACCTCTGTGACCAACTGTTGGA
GTTTAGTTTACATTGTAACATTCTATTGTAAAATCAACTAAAATTCAGACTTTTAGCAGGACTTTGTGTA
CAGTTAAAGGAGAGATGGCCAAGCCAGGGACAAATTGTCTATTAGAAAACGGTCCTAAGAGATTCTTTGG
TGTTTGGCACTTTAAGGTCATCGTTGGGCAGAAGTTTAGCATTAATAGTTGTTCTGAAACGTGTTTTATC
AGGTTTAGAGCCCATGTTGAGTCTTCTTTTCATGGGTTTTCATAATATTTTAAAACTATTTGTTTAGCGA
TGGTTTTGTTCGTTTAAGTAAAGGTTAATCTTGATGATATACATAATAATCTTTCTAAAATTGTATGCTG
ACCATACTTGCTGTCAGAATAATGCTAGGCATATGCTTTTTGCTAAATATGTATGTACAGAGTATTTGGA
AGTTAAGAATTGATTAGACTAGTGAATTTAGGAGTATTTGAGGIGGGIGGGGGGAAGAGGGAAATGACAA
CTGCAAATGTAGACTATACTGTAAAAATTCAGTTTGTTGCTTTAAAGAAACAAACTGATACCTGAATTTT
GCTGTGTTTCCATTTTTTAGAGATTTTTATCATTTTTTTCTCTCTCGGCATTCTTTTTTCTCATACTCTT
CAAAAAGCAGTTCTGCAGCTGGTTAATTCATGTAACTGTGAGAGCAAATGAATAATTCCTGCTATTCTGA
AATTGCCTACATGTTTCAATACCAGTTATATGGAGTGCTTGAATTTAATAAGCAGTTTTTACGGAGTTTA
CAGTACAGAAATAGGCTTTAATTTTCAAGTGAATTTTTTGCCAAACTTAGTAACTCTGTTAAATATTTGG
AGGATTTAAAGAACATCCCAGTTTGAATTCATTTCAAACTTTTTAAATTTTTTTGTACTATGTTTGGTTT
TATTTTCCTTCTGTTAATCTTTTGTATTCACTTATGCTCTCGTACATTGAGTACTTTTATTCCAAAACTA
GTGGGTTTTCTCTACTGGAAATTTTCAATAAACCTGTCATTATTGCTTACTTTGATTAAAAA
NM_001003688.1 Homo sapiens SMAD family member 1 (SMAD1), transcript
variant 2, mRNA (SEQ ID NO: 5)
CACTGCATGTGTATTCGTGAGTTCGCGGTTGAACAACTGTTCCTTTACTCTGCTCCCTGTCTTTGTGCTG
ACTGGGTTACTTTTTTAAACACTAGGAATGGTAATTTCTACTCTTCTGGACTTCAAACTAAGAAGTTAAA
GAGACTTCTCTGTAAATAAACAAATCTCTTCTGCTGTCCTTTTGCATTTGGAGACAGCTTTATTTCACCA
TATCCAAGGAGTATAACTAGTGCTGTCATTATGAATGTGACAAGTTTATTTTCCTTTACAAGTCCAGCTG
TGAAGAGACTTCTTGGGTGGAAACAGGGCGATGAAGAAGAAAAATGGGCAGAGAAAGCTGTTGATGCTTT
GGTGAAAAAACTGAAGAAAAAGAAAGGTGCCATGGAGGAACTGGAAAAGGCCTTGAGCTGCCCAGGGCAA
CCGAGTAACTGTGTCACCATTCCCCGCTCTCTGGATGGCAGGCTGCAAGTCTCCCACCGGAAGGGACTGC
CTCATGTCATTTACTGCCGTGTGTGGCGCTGGCCCGATCTTCAGAGCCACCATGAACTAAAACCACTGGA
ATGCTGTGAGTTTCCTTTTGGTTCCAAGCAGAAGGAGGTCTGCATCAATCCCTACCACTATAAGAGAGTA
GAAAGCCCTGTACTTCCTCCTGTGCTGGTTCCAAGACACAGCGAATATAATCCTCAGCACAGCCTCTTAG
CTCAGTTCCGTAACTTAGGACAAAATGAGCCTCACATGCCACTCAACGCCACTTTTCCAGATTCTTTCCA
GCAACCCAACAGCCACCCGTTTCCTCACTCTCCCAATAGCAGTTACCCAAACTCTCCTGGGAGCAGCAGC
AGCACCTACCCTCACTCTCCCACCAGCTCAGACCCAGGAAGCCCTTTCCAGATGCCAGCTGATACGCCCC
CACCTGCTTACCTGCCTCCTGAAGACCCCATGACCCAGGATGGCTCTCAGCCGATGGACACAAACATGAT
GGCGCCTCCCCTGCCCTCAGAAATCAACAGAGGAGATGTTCAGGCGGTTGCTTATGAGGAACCAAAACAC
TGGTGCTCTATTGTCTACTATGAGCTCAACAATCGTGTGGGTGAAGCGTTCCATGCCTCCTCCACAAGTG
TGTTGGTGGATGGTTTCACTGATCCTTCCAACAATAAGAACCGTTTCTGCCTTGGGCTGCTCTCCAATGT
TAACCGGAATTCCACTATTGAAAACACCAGGCGGCATATTGGAAAAGGAGTTCATCTTTATTATGTTGGA
GGGGAGGTGTATGCCGAATGCCTTAGTGACAGTAGCATCTTTGTGCAAAGTCGGAACTGCAACTACCATC
ATGGATTTCATCCTACTACTGTTTGCAAGATCCCTAGTGGGTGTAGTCTGAAAATTTTTAACAACCAAGA
ATTTGCTCAGTTATTGGCACAGTCTGTGAACCATGGATTTGAGACAGTCTATGAGCTTACAAAAATGTGT
ACTATACGTATGAGCTTTGTGAAGGGCTGGGGAGCAGAATACCACCGCCAGGATGTTACTAGCACCCCCT
GCTGGATTGAGATACATCTGCACGGCCCCCTCCAGTGGCTGGATAAAGTTCTTACTCAAATGGGTTCACC
TCATAATCCTATTTCATCTGTATCTTAAATGGCCCCAGGCATCTGCCTCTGGAAAACTATTGAGCCTTGC
ATGTACTTGAAGGATGGATGAGTCAGACACGATTGAGAACTGACAAAGGAGCCTTGATAATACTTGACCT
CTGTGACCAACTGTTGGATTCAGAAATTTAAACAAAAAAAAAAAAAAACACACACACCTTGGTAACATAC
TGTTGATATCAAGAACCTGTTTAGTTTACATTGTAACATTCTATTGTAAAATCAACTAAAATTCAGACTT
TCTGAAACGTGTTTTATCAGGTTTAGAGCCCATGTTGAGTCTTCTTTTCATGGGTTTTCATAATATTTTA
AAACTATTTGTTTAGCGATGGTTTTGTTCGTTTAAGTAAAGGTTAATCTTGATGATATACATAATAATCT
TTCTAAAATTGTATGCTGACCATACTTGCTGTCAGAATAATGCTAGGCATATGCTTTTTGCTAAATATGT
ATGTACAGAGTATTTGGAAGTTAAGAATTGATTAGACTAGTGAATTTAGGAGTATTTGAGGIGGGTGGGG
GGAAGAGGGAAATGACAACTGCAAATGTAGACTATACTGTAAAAATTCAGTTTGTTGCTTTAAAGAAACA
AACTGATACCTGAATTTTGCTGTGTTTCCATTTTTTAGAGATTTTTATCATTTTTTTCTCTCTCGGCATT
CTTTTTTCTCATACTCTTCAAAAAGCAGTTCTGCAGCTGGTTAATTCATGTAACTGTGAGAGCAAATGAA
TAATTCCTGCTATTCTGAAATTGCCTACATGTTTCAATACCAGTTATATGGAGTGCTTGAATTTAATAAG
CAGTTTTTACGGAGTTTACAGTACAGAAATAGGCTTTAATTTTCAAGTGAATTTTTTGCCAAACTTAGTA
ACTCTGTTAAATATTTGGAGGATTTAAAGAACATCCCAGTTTGAATTCATTTCAAACTTTTTAAATTTTT
TTGTACTATGTTTGGTTTTATTTTCCTTCTGTTAATCTTTTGTATTCACTTATGCTCTCGTACATTGAGT
ACTTTTATTCCAAAACTAGTGGGTTTTCTCTACTGGAAATTTTCAATAAACCTGTCATTATTGCTTACTT
TGATTAAAAA
NM_001354811.1 Homo sapiens SMAD family member 1 (SMAD1), transcript
variant 3, mRNA (SEQ ID NO: 6)
GCTGTGGGAAGCCCAGTTCCCGGGCCCCCGAGCCTCGGCTCCCGGGCCTGACCGCGCTGGGATCTCCCCG
GCCGCGCTCCCCTTCCGCGCGCTCCTCACATCTCTCCCGTGCTGCCGCCGGGCCGAGGCCCGTTCGCGTG
GCCCGCGGACCCATTGTGTCCCCCGCGCCGGCGGGGCGACCCCTGCGGGAGCTGGAGGACGACCGCTGGC
GCTGCTCTCCAAGGCGCCTGGTGGAGCGGGTCTCGCGGGCGGGGGACCCCGGCGCCCCGGGCCCCTCCAC
ATCCCGCACGGGTTTTCTTCTCGGCCCCAGCAAGCCTCTTTGGGGTCGAGGTCAAGGAAAGTTCGCACCG
AGATCCCCTCTAATTTATTCAAAGGTTTGGCGGCGGCGCGTAATTTTTTCCCCCTCTTCCGCCTACACCC
GCTGCGTCTCCTGGTGTCTCGTTCCTTTCCCTTTACCGGAGTCGATTGCCTCACTGCATGTGTATTCGTG
CTGACTGGGTTACTTTTTTAAACACTAGGAATGGTAATTTCTACTCTTCTGGACTTCAAACTAAGAAGTT
AAAGAGACTTCTCTGTAAATAAACAAATCTCTTCTGCTGTCCTTTTGCATTTGGAGACAGCTTTATTTCA
CCATATCCAAGGAGTATAACTAGTGCTGTCATTATGAATGTGACAAGTTTATTTTCCTTTACAAGTCCAG
CTGTGAAGAGACTTCTTGGGTGGAAACAGGGCGATGAAGAAGAAAAATGGGCAGAGAAAGCTGTTGATGC
TTTGGTGAAAAAACTGAAGAAAAAGAAAGGTGCCATGGAGGAACTGGAAAAGGCCTTGAGCTGCCCAGGG
CAACCGAGTAACTGTGTCACCATTCCCCGCTCTCTGGATGGCAGGCTGCAAGTCTCCCACCGGAAGGGAC
TGCCTCATGTCATTTACTGCCGTGTGTGGCGCTGGCCCGATCTTCAGAGCCACCATGAACTAAAACCACT
GGAATGCTGTGAGTTTCCTTTTGGTTCCAAGCAGAAGGAGGTCTGCATCAATCCCTACCACTATAAGAGA
GTAGAAAGCCCTGTACTTCCTCCTGTGCTGGTTCCAAGACACAGCGAATATAATCCTCAGCACAGCCTCT
TAGCTCAGTTCCGTAACTTAGGACAAAATGAGCCTCACATGCCACTCAACGCCACTTTTCCAGATTCTTT
CCAGCAACCCAACAGCCACCCGTTTCCTCACTCTCCCAATAGCAGTTACCCAAACTCTCCTGGGAGCAGC
AGCAGCACCTACCCTCACTCTCCCACCAGCTCAGACCCAGGAAGCCCTTTCCAGATGCCAGCTGATACGC
CCCCACCTGCTTACCTGCCTCCTGAAGACCCCATGACCCAGGATGGCTCTCAGCCGATGGACACAAACAT
GATGGCGCCTCCCCTGCCCTCAGAAATCAACAGAGGAGATGTTCAGGCGGTTGCTTATGAGGAACCAAAA
CACTGGTGCTCTATTGTCTACTATGAGCTCAACAATCGTGTGGGTGAAGCGTTCCATGCCTCCTCCACAA
GTGTGTTGGTGGATGGTTTCACTGATCCTTCCAACAATAAGAACCGTTTCTGCCTTGGGCTGCTCTCCAA
TGTTAACCGGAATTCCACTATTGAAAACACCAGGCGGCATATTGGAAAAGGAGTTCATCTTTATTATGTT
GGAGGGGAGGTGTATGCCGAATGCCTTAGTGACAGTAGCATCTTTGTGCAAAGTCGGAACTGCAACTACC
ATCATGGATTTCATCCTACTACTGTTTGCAAGATCCCTAGTGGGTGTAGTCTGAAAATTTTTAACAACCA
AGAATTTGCTCAGTTATTGGCACAGTCTGTGAACCATGGATTTGAGACAGTCTATGAGCTTACAAAAATG
TGTACTATACGTATGAGCTTTGTGAAGGGCTGGGGAGCAGAATACCACCGCCAGGATGTTACTAGCACCC
CCTGCTGGATTGAGATACATCTGCACGGCCCCCTCCAGTGGCTGGATAAAGTTCTTACTCAAATGGGTTC
ACCTCATAATCCTATTTCATCTGTATCTTAAATGGCCCCAGGCATCTGCCTCTGGAAAACTATTGAGCCT
TGCATGTACTTGAAGGATGGATGAGTCAGACACGATTGAGAACTGACAAAGGAGCCTTGATAATACTTGA
CCTCTGTGACCAACTGTTGGATTCAGAAATTTAAACAAAAAAAAAAAAAAACACACACACCTTGGTAACA
TACTGTTGATATCAAGAACCTGTTTAGTTTACATTGTAACATTCTATTGTAAAATCAACTAAAATTCAGA
CTTTTAGCAGGACTTTGTGTACAGTTAAAGGAGAGATGGCCAAGCCAGGGACAAATTGTCTATTAGAAAA
CGGTCCTAAGAGATTCTTTGGTGTTTGGCACTTTAAGGTCATCGTTGGGCAGAAGTTTAGCATTAATAGT
TGTTCTGAAACGTGTTTTATCAGGTTTAGAGCCCATGTTGAGTCTTCTTTTCATGGGTTTTCATAATATT
TTAAAACTATTTGTTTAGCGATGGTTTTGTTCGTTTAAGTAAAGGTTAATCTTGATGATATACATAATAA
TCTTTCTAAAATTGTATGCTGACCATACTTGCTGTCAGAATAATGCTAGGCATATGCTTTTTGCTAAATA
TGTATGTACAGAGTATTTGGAAGTTAAGAATTGATTAGACTAGTGAATTTAGGAGTATTTGAGGTGGGTG
GGGGGAAGAGGGAAATGACAACTGCAAATGTAGACTATACTGTAAAAATTCAGTTTGTTGCTTTAAAGAA
ACAAACTGATACCTGAATTTTGCTGTGTTTCCATTTTTTAGAGATTTTTATCATTTTTTTCTCTCTCGGC
ATTCTTTTTTCTCATACTCTTCAAAAAGCAGTTCTGCAGCTGGTTAATTCATGTAACTGTGAGAGCAAAT
GAATAATTCCTGCTATTCTGAAATTGCCTACATGTTTCAATACCAGTTATATGGAGTGCTTGAATTTAAT
AAGCAGTTTTTACGGAGTTTACAGTACAGAAATAGGCTTTAATTTTCAAGTGAATTTTTTGCCAAACTTA
GTAACTCTGTTAAATATTTGGAGGATTTAAAGAACATCCCAGTTTGAATTCATTTCAAACTTTTTAAATT
TTTTTGTACTATGTTTGGTTTTATTTTCCTTCTGTTAATCTTTTGTATTCACTTATGCTCTCGTACATTG
AGTACTTTTATTCCAAAACTAGTGGGTTTTCTCTACTGGAAATTTTCAATAAACCTGTCATTATTGCTTA
CTTTGATTAAAAA
NM_001354812.1 Homo sapiens SMAD family member 1 (SMAD1), transcript
variant 4, mRNA (SEQ ID NO: 7)
CAATTCTGGGTACGTACAACTTCTGGGGCCTGCAAATTATTGGAGAGTGAGTGAGGGGCAACGAAAGATA
GACATAAAAGGGCGCGTCTCGAAAGGTGCTGACTGGGTTACTTTTTTAAACACTAGGAATGGTAATTTCT
ACTCTTCTGGACTTCAAACTAAGAAGTTAAAGAGACTTCTCTGTAAATAAACAAATCTCTTCTGCTGTCC
ACAAGTTTATTTTCCTTTACAAGTCCAGCTGTGAAGAGACTTCTTGGGTGGAAACAGGGCGATGAAGAAG
AAAAATGGGCAGAGAAAGCTGTTGATGCTTTGGTGAAAAAACTGAAGAAAAAGAAAGGTGCCATGGAGGA
ACTGGAAAAGGCCTTGAGCTGCCCAGGGCAACCGAGTAACTGTGTCACCATTCCCCGCTCTCTGGATGGC
AGGCTGCAAGTCTCCCACCGGAAGGGACTGCCTCATGTCATTTACTGCCGTGTGTGGCGCTGGCCCGATC
TTCAGAGCCACCATGAACTAAAACCACTGGAATGCTGTGAGTTTCCTTTTGGTTCCAAGCAGAAGGAGGT
CTGCATCAATCCCTACCACTATAAGAGAGTAGAAAGCCCTGTACTTCCTCCTGTGCTGGTTCCAAGACAC
AGCGAATATAATCCTCAGCACAGCCTCTTAGCTCAGTTCCGTAACTTAGGACAAAATGAGCCTCACATGC
CACTCAACGCCACTTTTCCAGATTCTTTCCAGCAACCCAACAGCCACCCGTTTCCTCACTCTCCCAATAG
CAGTTACCCAAACTCTCCTGGGAGCAGCAGCAGCACCTACCCTCACTCTCCCACCAGCTCAGACCCAGGA
AGCCCTTTCCAGATGCCAGCTGATACGCCCCCACCTGCTTACCTGCCTCCTGAAGACCCCATGACCCAGG
ATGGCTCTCAGCCGATGGACACAAACATGATGGCGCCTCCCCTGCCCTCAGAAATCAACAGAGGAGATGT
TCAGGCGGTTGCTTATGAGGAACCAAAACACTGGTGCTCTATTGTCTACTATGAGCTCAACAATCGTGTG
GGTGAAGCGTTCCATGCCTCCTCCACAAGTGTGTTGGTGGATGGTTTCACTGATCCTTCCAACAATAAGA
ACCGTTTCTGCCTTGGGCTGCTCTCCAATGTTAACCGGAATTCCACTATTGAAAACACCAGGCGGCATAT
TGGAAAAGGAGTTCATCTTTATTATGTTGGAGGGGAGGTGTATGCCGAATGCCTTAGTGACAGTAGCATC
TTTGTGCAAAGTCGGAACTGCAACTACCATCATGGATTTCATCCTACTACTGTTTGCAAGATCCCTAGTG
GGTGTAGTCTGAAAATTTTTAACAACCAAGAATTTGCTCAGTTATTGGCACAGTCTGTGAACCATGGATT
TGAGACAGTCTATGAGCTTACAAAAATGTGTACTATACGTATGAGCTTTGTGAAGGGCTGGGGAGCAGAA
TACCACCGCCAGGATGTTACTAGCACCCCCTGCTGGATTGAGATACATCTGCACGGCCCCCTCCAGTGGC
TGGATAAAGTTCTTACTCAAATGGGTTCACCTCATAATCCTATTTCATCTGTATCTTAAATGGCCCCAGG
CATCTGCCTCTGGAAAACTATTGAGCCTTGCATGTACTTGAAGGATGGATGAGTCAGACACGATTGAGAA
CTGACAAAGGAGCCTTGATAATACTTGACCTCTGTGACCAACTGTTGGATTCAGAAATTTAAACAAAAAA
AAAAAAAAACACACACACCTTGGTAACATACTGTTGATATCAAGAACCTGTTTAGTTTACATTGTAACAT
TCTATTGTAAAATCAACTAAAATTCAGACTTTTAGCAGGACTTTGTGTACAGTTAAAGGAGAGATGGCCA
AGCCAGGGACAAATTGTCTATTAGAAAACGGTCCTAAGAGATTCTTTGGTGTTTGGCACTTTAAGGTCAT
TCTTCTTTTCATGGGTTTTCATAATATTTTAAAACTATTIGTTTAGCGATGGTTTTGTTCGTTTAAGTAA
AGGTTAATCTTGATGATATACATAATAATCTTTCTAAAATTGTATGCTGACCATACTTGCTGTCAGAATA
ATGCTAGGCATATGCTTTTTGCTAAATATGTATGTACAGAGTATTTGGAAGTTAAGAATTGATTAGACTA
GTGAATTTAGGAGTATTTGAGGTGGGTGGGGGGAAGAGGGAAATGACAACTGCAAATGTAGACTATACTG
TAAAAATTCAGTTTGTTGCTTTAAAGAAACAAACTGATACCTGAATTTTGCTGTGTTTCCATTTTTTAGA
GATTTTTATCATTTTTTTCTCTCTCGGCATTCTTTTTTCTCATACTCTTCAAAAAGCAGTTCTGCAGCTG
GTTAATTCATGTAACTGTGAGAGCAAATGAATAATTCCTGCTATTCTGAAATTGCCTACATGTTTCAATA
CCAGTTATATGGAGTGCTTGAATTTAATAAGCAGTTTTTACGGAGTTTACAGTACAGAAATAGGCTTTAA
TTTTCAAGTGAATTTTTTGCCAAACTTAGTAACTCTGTTAAATATTTGGAGGATTTAAAGAACATCCCAG
TTGTATTCACTTATGCTCTCGTACATTGAGTACTTTTATTCCAAAACTAGTGGGTTTTCTCTACTGGAAA
TTTTCAATAAACCTGTCATTATTGCTTACTTTGATTAAAAA
NM_001354813.1 Homo sapiens SMAD family member 1 (SMAD1), transcript
variant 5, mRNA (SEQ ID NO: 8)
GCCGTCCTCCGGCCCCGGCCGCGCTGCGCTCACGCCGGCCGGGCCGGGAATTTGGAGAGGATCCCTGGTC
GCGCGGCAGCGGCGGCGGCGCGCGGGTGAGCGGGTGCTGACTGGGTTACTTTTTTAAACACTAGGAATGG
TAATTTCTACTCTTCTGGACTTCAAACTAAGAAGTTAAAGAGACTTCTCTGTAAATAAACAAATCTCTTC
TGAATGTGACAAGTTTATTTTCCTTTACAAGTCCAGCTGTGAAGAGACTTCTTGGGTGGAAACAGGGCGA
TGAAGAAGAAAAATGGGCAGAGAAAGCTGTTGATGCTTTGGTGAAAAAACTGAAGAAAAAGAAAGGTGCC
ATGGAGGAACTGGAAAAGGCCTTGAGCTGCCCAGGGCAACCGAGTAACTGTGTCACCATTCCCCGCTCTC
TGGATGGCAGGCTGCAAGTCTCCCACCGGAAGGGACTGCCTCATGTCATTTACTGCCGTGTGTGGCGCTG
GCCCGATCTTCAGAGCCACCATGAACTAAAACCACTGGAATGCTGTGAGTTTCCTTTTGGTTCCAAGCAG
AAGGAGGTCTGCATCAATCCCTACCACTATAAGAGAGTAGAAAGCCCTGTACTTCCTCCTGTGCTGGTTC
CAAGACACAGCGAATATAATCCTCAGCACAGCCTCTTAGCTCAGTTCCGTAACTTAGGACAAAATGAGCC
TCACATGCCACTCAACGCCACTTTTCCAGATTCTTTCCAGCAACCCAACAGCCACCCGTTTCCTCACTCT
CCCAATAGCAGTTACCCAAACTCTCCTGGGAGCAGCAGCAGCACCTACCCTCACTCTCCCACCAGCTCAG
ACCCAGGAAGCCCTTTCCAGATGCCAGCTGATACGCCCCCACCTGCTTACCTGCCTCCTGAAGACCCCAT
GACCCAGGATGGCTCTCAGCCGATGGACACAAACATGATGGCGCCTCCCCTGCCCTCAGAAATCAACAGA
GGAGATGTTCAGGCGGTTGCTTATGAGGAACCAAAACACTGGTGCTCTATTGTCTACTATGAGCTCAACA
ATCGTGTGGGTGAAGCGTTCCATGCCTCCTCCACAAGTGTGTTGGTGGATGGTTTCACTGATCCTTCCAA
CAATAAGAACCGTTTCTGCCTTGGGCTGCTCTCCAATGTTAACCGGAATTCCACTATTGAAAACACCAGG
CGGCATATTGGAAAAGGAGTTCATCTTTATTATGTTGGAGGGGAGGTGTATGCCGAATGCCTTAGTGACA
GTAGCATCTTTGTGCAAAGTCGGAACTGCAACTACCATCATGGATTTCATCCTACTACTGTTTGCAAGAT
CCCTAGTGGGTGTAGTCTGAAAATTTTTAACAACCAAGAATTTGCTCAGTTATTGGCACAGTCTGTGAAC
CATGGATTIGAGACAGTCTATGAGCTTACAAAAATGTGTACTATACGTATGAGCTTTGTGAAGGGCTGGG
GAGCAGAATACCACCGCCAGGATGTTACTAGCACCCCCTGCTGGATTGAGATACATCTGCACGGCCCCCT
CCAGTGGCTGGATAAAGTTCTTACTCAAATGGGTTCACCTCATAATCCTATTTCATCTGTATCTTAAATG
GCCCCAGGCATCTGCCTCTGGAAAACTATTGAGCCTTGCATGTACTTGAAGGATGGATGAGTCAGACACG
ATTGAGAACTGACAAAGGAGCCTTGATAATACTTGACCTCTGTGACCAACTGTTGGATTCAGAAATTTAA
TGTAACATTCTATTGTAAAATCAACTAAAATTCAGACTTTTAGCAGGACTTTGTGTACAGTTAAAGGAGA
GATGGCCAAGCCAGGGACAAATTGTCTATTAGAAAACGGTCCTAAGAGATTCTTTGGTGTTTGGCACTTT
AAGGTCATCGTTGGGCAGAAGTTTAGCATTAATAGTTGTTCTGAAACGTGTTTTATCAGGTTTAGAGCCC
ATGTTGAGTCTTCTTTTCATGGGTTTTCATAATATTTTAAAACTATTTGTTTAGCGATGGTTTTGTTCGT
TTAAGTAAAGGTTAATCTTGATGATATACATAATAATCTTTCTAAAATTGTATGCTGACCATACTTGCTG
TCAGAATAATGCTAGGCATATGCTTTTTGCTAAATATGTATGTACAGAGTATTTGGAAGTTAAGAATTGA
TTAGACTAGTGAATTTAGGAGTATTTGAGGTGGGTGGGGGGAAGAGGGAAATGACAACTGCAAATGTAGA
CTATACTGTAAAAATTCAGTTTGTTGCTTTAAAGAAACAAACTGATACCTGAATTTTGCTGTGTTTCCAT
TGCAGCTGGTTAATTCATGTAACTGTGAGAGCAAATGAATAATTCCTGCTATTCTGAAATTGCCTACATG
TTTCAATACCAGTTATATGGAGTGCTTGAATTTAATAAGCAGTTTTTACGGAGTTTACAGTACAGAAATA
GGCTTTAATTTTCAAGTGAATTTTTTGCCAAACTTAGTAACTCTGTTAAATATTTGGAGGATTTAAAGAA
CATCCCAGTTTGAATTCATTTCAAACTTTTTAAATTTTTTTGTACTATGTTTGGTTTTATTTTCCTTCTG
TTAATCTTTTGTATTCACTTATGCTCTCGTACATTGAGTACTTTTATTCCAAAACTAGTGGGTTTTCTCT
ACTGGAAATTTTCAATAAACCTGTCATTATTGCTTACTTTGATTAAAAA
NM_001354814.1 Homo sapiens SMAD family member 1 (SMAD1), transcript
variant 6, mRNA (SEQ ID NO: 9)
GCCGTCCTCCGGCCCCGGCCGCGCTGCGCTCACGCCGGCCGGGCCGGGAATTTGGAGAGGATCCCTGGTC
GCGCGGCAGCGGCGGCGGCGCGCGGGTGCTGACTGGGTTACTTTTTTAAACACTAGGAATGGTAATTTCT
ACTCTTCTGGACTTCAAACTAAGAAGTTAAAGAGACTTCTCTGTAAATAAACAAATCTCTTCTGCTGTCC
ACAAGTTTATTTTCCTTTACAAGTCCAGCTGTGAAGAGACTTCTTGGGTGGAAACAGGGCGATGAAGAAG
AAAAATGGGCAGAGAAAGCTGTTGATGCTTTGGTGAAAAAACTGAAGAAAAAGAAAGGTGCCATGGAGGA
ACTGGAAAAGGCCTTGAGCTGCCCAGGGCAACCGAGTAACTGTGTCACCATTCCCCGCTCTCTGGATGGC
AGGCTGCAAGTCTCCCACCGGAAGGGACTGCCTCATGTCATTTACTGCCGTGTGTGGCGCTGGCCCGATC
TTCAGAGCCACCATGAACTAAAACCACTGGAATGCTGTGAGTTTCCTTTTGGTTCCAAGCAGAAGGAGGT
CTGCATCAATCCCTACCACTATAAGAGAGTAGAAAGCCCTGTACTTCCTCCTGTGCTGGTTCCAAGACAC
AGCGAATATAATCCTCAGCACAGCCTCTTAGCTCAGTTCCGTAACTTAGGACAAAATGAGCCTCACATGC
CACTCAACGCCACTTTTCCAGATTCTTTCCAGCAACCCAACAGCCACCCGTTTCCTCACTCTCCCAATAG
CAGTTACCCAAACTCTCCTGGGAGCAGCAGCAGCACCTACCCTCACTCTCCCACCAGCTCAGACCCAGGA
AGCCCTTTCCAGATGCCAGCTGATACGCCCCCACCTGCTTACCTGCCTCCTGAAGACCCCATGACCCAGG
ATGGCTCTCAGCCGATGGACACAAACATGATGGCGCCTCCCCTGCCCTCAGAAATCAACAGAGGAGATGT
TCAGGCGGTTGCTTATGAGGAACCAAAACACTGGTGCTCTATTGTCTACTATGAGCTCAACAATCGTGTG
GGTGAAGCGTTCCATGCCTCCTCCACAAGTGTGTTGGTGGATGGTTTCACTGATCCTTCCAACAATAAGA
ACCGTTTCTGCCTTGGGCTGCTCTCCAATGTTAACCGGAATTCCACTATTGAAAACACCAGGCGGCATAT
TGGAAAAGGAGTTCATCTTTATTATGTTGGAGGGGAGGTGTATGCCGAATGCCTTAGTGACAGTAGCATC
TTTGTGCAAAGTCGGAACTGCAACTACCATCATGGATTTCATCCTACTACTGTTTGCAAGATCCCTAGTG
GGTGTAGTCTGAAAATTTTTAACAACCAAGAATTTGCTCAGTTATTGGCACAGTCTGTGAACCATGGATT
TGAGACAGTCTATGAGCTTACAAAAATGTGTACTATACGTATGAGCTTTGTGAAGGGCTGGGGAGCAGAA
TACCACCGCCAGGATGTTACTAGCACCCCCTGCTGGATTGAGATACATCTGCACGGCCCCCTCCAGTGGC
TGGATAAAGTTCTTACTCAAATGGGTTCACCTCATAATCCTATTTCATCTGTATCTTAAATGGCCCCAGG
CATCTGCCTCTGGAAAACTATTGAGCCTTGCATGTACTTGAAGGATGGATGAGTCAGACACGATTGAGAA
CTGACAAAGGAGCCTTGATAATACTTGACCTCTGTGACCAACTGTTGGATTCAGAAATTTAAACAAAAAA
AAAAAAAAACACACACACCTTGGTAACATACTGTTGATATCAAGAACCTGTTTAGTTTACATTGTAACAT
TCTATTGTAAAATCAACTAAAATTCAGACTTTTAGCAGGACTTTGTGTACAGTTAAAGGAGAGATGGCCA
AGCCAGGGACAAATTGTCTATTAGAAAACGGTCCTAAGAGATTCTTTGGTGTTTGGCACTTTAAGGTCAT
CGTTGGGCAGAAGTTTAGCATTAATAGTTGTTCTGAAACGTGTTTTATCAGGTTTAGAGCCCATGTTGAG
TCTTCTTTTCATGGGTTTTCATAATATTTTAAAACTATTTGTTTAGCGATGGTTTTGTTCGTTTAAGTAA
AGGTTAATCTTGATGATATACATAATAATCTTTCTAAAATTGTATGCTGACCATACTTGCTGTCAGAATA
ATGCTAGGCATATGCTTTTTGCTAAATATGTATGTACAGAGTATTTGGAAGTTAAGAATTGATTAGACTA
GTGAATTTAGGAGTATTTGAGGTGGGTGGGGGGAAGAGGGAAATGACAACTGCAAATGTAGACTATACTG
TAAAAATTCAGTTTGTTGCTTTAAAGAAACAAACTGATACCTGAATTTTGCTGTGTTTCCATTTTTTAGA
GATTTTTATCATTTTTTTCTCTCTCGGCATTCTTTTTTCTCATACTCTTCAAAAAGCAGTTCTGCAGCTG
GTTAATTCATGTAACTGTGAGAGCAAATGAATAATTCCTGCTATTCTGAAATTGCCTACATGTTTCAATA
CCAGTTATATGGAGTGCTTGAATTTAATAAGCAGTTTTTACGGAGTTTACAGTACAGAAATAGGCTTTAA
TTTTCAAGTGAATTTTTTGCCAAACTTAGTAACTCTGTTAAATATTTGGAGGATTTAAAGAACATCCCAG
TTTGAATTCATTTCAAACTTTTTAAATTTTTTTGTACTATGTTTGGTTTTATTTTCCTTCTGTTAATCTT
TTGTATTCACTTATGCTCTCGTACATTGAGTACTTTTATTCCAAAACTAGTGGGTTTTCTCTACTGGAAA
TTTTCAATAAACCTGTCATTATTGCTTACTTTGATTAAAAA
NM_001354816.1 Homo sapiens SMAD family member 1 (SMAD1), transcript
variant 7, mRNA (SEQ ID NO: 10)
GCTGTGGGAAGCCCAGTTCCCGGGCCCCCGAGCCTCGGCTCCCGGGCCTGACCGCGCTGGGATCTCCCCG
GCCGCGCTCCCCTTCCGCGCGCTCCTCACATCTCTCCCGTGCTGCCGCCGGGCCGAGGCCCGTTCGCGTG
GCCCGCGGACCCATTGTGTCCCCCGCGCCGGCGGGGCGACCCCTGCGGGAGCTGGAGGACGACCGCTGGC
GCTGCTCTCCAAGGCGCCTGGTGGAGCGGGTCTCGCGGGCGGGGGACCCCGGCGCCCCGGGCCCCTCCAC
ATCCCGCACGGGTTTTCTTCTCGGCCCCAGCAAGCCTCTTTGGGGTCGAGGTCAAGGAAAGTTCGCACCG
AGATCCCCTCTAATTTATTCAAAGGTGCTGACTGGGTTACTTTTTTAAACACTAGGAATGGTAATTTCTA
CTCTTCTGGACTTCAAACTAAGAAGTTAAAGAGACTTCTCTGTAAATAAACAAATCTCTTCTGCTGTCCT
TTTGCATTTGGAGACAGCTTTATTICACCATATCCAAGGAGTATAACTAGTGCTGTCATTATGAATGTGA
CAAGTTTATTTTCCTTTACAAGTCCAGCTGTGAAGAGACTTCTTGGGTGGAAACAGGGCGATGAAGAAGA
AAAATGGGCAGAGAAAGCTGTTGATGCTTTGGTGAAAAAACTGAAGAAAAAGAAAGGTGCCATGGAGGAA
CTGGAAAAGGCCTTGAGCTGCCCAGGGCAACCGAGTAACTGTGTCACCATTCCCCGCTCTCTGGATGGCA
GGCTGCAAGTCTCCCACCGGAAGGGACTGCCTCATGTCATTTACTGCCGTGTGTGGCGCTGGCCCGATCT
TCAGAGCCACCATGAACTAAAACCACTGGAATGCTGTGAGTTTCCTTTTGGTTCCAAGCAGAAGGAGGTC
TGCATCAATCCCTACCACTATAAGAGAGTAGAAAGCCCTGTACTTCCTCCTGTGCTGGTTCCAAGACACA
GCGAATATAATCCTCAGCACAGCCTCTTAGCTCAGTTCCGTAACTTAGGACAAAATGAGCCTCACATGCC
ACTCAACGCCACTTTTCCAGATTCTTTCCAGCAACCCAACAGCCACCCGTTTCCTCACTCTCCCAATAGC
AGTTACCCAAACTCTCCTGGGAGCAGCAGCAGCACCTACCCTCACTCTCCCACCAGCTCAGACCCAGGAA
GCCCTTTCCAGATGCCAGCTGATACGCCCCCACCTGCTTACCTGCCTCCTGAAGACCCCATGACCCAGGA
TGGCTCTCAGCCGATGGACACAAACATGATGGCGCCTCCCCTGCCCTCAGAAATCAACAGAGGAGATGTT
CAGGCGGTTGCTTATGAGGAACCAAAACACTGGTGCTCTATTGTCTACTATGAGCTCAACAATCGTGTGG
GTGAAGCGTTCCATGCCTCCTCCACAAGTGTGTTGGTGGATGGTTTCACTGATCCTTCCAACAATAAGAA
CCGTTTCTGCCTTGGGCTGCTCTCCAATGTTAACCGGAATTCCACTATTGAAAACACCAGGCGGCATATT
GGAAAAGGAGTTCATCTTTATTATGTTGGAGGGGAGGTGTATGCCGAATGCCTTAGTGACAGTAGCATCT
TTGTGCAAAGTCGGAACTGCAACTACCATCATGGATTTCATCCTACTACTGTTTGCAAGATCCCTAGTGG
GTGTAGTCTGAAAATTTTTAACAACCAAGAATTTGCTCAGTTATTGGCACAGTCTGTGAACCATGGATTT
GAGACAGTCTATGAGCITACAAAAATGTGTACTATACGTATGAGCTTIGTGAAGGGCIGGGGAGCAGAAT
ACCACCGCCAGGATGTTACTAGCACCCCCTGCTGGATTGAGATACATCTGCACGGCCCCCTCCAGTGGCT
GGATAAAGTTCTTACTCAAATGGGTTCACCTCATAATCCTATTTCATCTGTATCTTAAATGGCCCCAGGC
ATCTGCCTCTGGAAAACTATTGAGCCTTGCATGTACTTGAAGGATGGATGAGTCAGACACGATTGAGAAC
TGACAAAGGAGCCTTGATAATACTTGACCTCTGTGACCAACTGTTGGATTCAGAAATTTAAACAAAAAAA
AAAAAAAACACACACACCTTGGTAACATACTGTTGATATCAAGAACCTGTTTAGTTTACATTGTAACATT
CTATTGTAAAATCAACTAAAATTCAGACTTTTAGCAGGACTTTGTGTACAGTTAAAGGAGAGATGGCCAA
GCCAGGGACAAATTGTCTATTAGAAAACGGTCCTAAGAGATTCTTTGGTGTTTGGCACTTTAAGGTCATC
GTTGGGCAGAAGTTTAGCATTAATAGTTGTTCTGAAACGTGTTTTATCAGGTTTAGAGCCCATGTTGAGT
CTTCTTTTCATGGGTTTTCATAATATTTTAAAACTATTTGTTTAGCGATGGTTTTGTTCGTTTAAGTAAA
GGTTAATCTTGATGATATACATAATAATCTTTCTAAAATTGTATGCTGACCATACTTGCTGTCAGAATAA
TGCTAGGCATATGCTTTTTGCTAAATATGTATGTACAGAGTATTTGGAAGTTAAGAATTGATTAGACTAG
TGAATTTAGGAGTATTTGAGGTGGGTGGGGGGAAGAGGGAAATGACAACTGCAAATGTAGACTATACTGT
AAAAATTCAGTTTGTTGCTTTAAAGAAACAAACTGATACCTGAATTTTGCTGTGTTTCCATTTTTTAGAG
ATTTTTATCATTTTTTTCTCTCTCGGCATTCTTTTTTCTCATACTCTTCAAAAAGCAGTTCTGCAGCTGG
TTAATTCATGTAACTGTGAGAGCAAATGAATAATTCCTGCTATTCTGAAATTGCCTACATGTTTCAATAC
CAGTTATATGGAGTGCTTGAATTTAATAAGCAGTTTTTACGGAGTTTACAGTACAGAAATAGGCTTTAAT
TTTCAAGTGAATTTTTTGCCAAACTTAGTAACTCTGTTAAATATTTGGAGGATTTAAAGAACATCCCAGT
TTGAATTCATTTCAAACTTTTTAAATTTTTTTGTACTATGTTTGGTTTTATTTTCCTTCTGTTAATCTTT
TGTATTCACTTATGCTCTCGTACATTGAGTACTTTTATTCCAAAACTAGTGGGTTTTCTCTACTGGAAAT
TTTCAATAAACCTGTCATTATTGCTTACTTTGATTAAAAA
NM_001354817.1 Homo sapiens SMAD family member 1 (SMAD1), transcript
variant 8, mRNA (SEQ ID NO: 11)
CACTGCATGTGTATTCGTGAGTTCGCGGTTGAACAACTGTTCCTTTACTCTGCTCCCTGTCTTTGTTAGT
GTTTCTCGGGGTTGTTTCTGTAGGAAGGTGGGGGTGGTGGGCGTGAGAGACAGATGTGGGCTTGTTTTTC
TAGTTGCTGAAACTGTATGAAGGCTTTAAAGGGAGAACGTTTTCTTGATGTGCTTTAGGAGGGGAGGAGG
AACAAATGCCTGCCAGATCTCACAGCTACAGTAGCTGAGCTTTTGTTTATTTTGAAGAGCATGCAATTTT
TAAATACACGGTGCAAGATAACCAGTAAAGGCGCGTTCCTTCTGAAAATTGAGGCCGGTCTCAGAACCAT
CTCCTGAGAAAGCATCCTTTTCGTGCTGACTGGGTTACTTTTTTAAACACTAGGAATGGTAATTTCTACT
CTTCTGGACTTCAAACTAAGAAGTTAAAGAGACTTCTCTGTAAATAAACAAATCTCTTCTGCTGTCCTTT
AGTTTATTTTCCTTTACAAGTCCAGCTGTGAAGAGACTTCTTGGGTGGAAACAGGGCGATGAAGAAGAAA
AATGGGCAGAGAAAGCTGTTGATGCTTTGGTGAAAAAACTGAAGAAAAAGAAAGGTGCCATGGAGGAACT
GGAAAAGGCCTTGAGCTGCCCAGGGCAACCGAGTAACTGTGTCACCATTCCCCGCTCTCTGGATGGCAGG
CTGCAAGTCTCCCACCGGAAGGGACTGCCTCATGTCATTTACTGCCGTGTGTGGCGCTGGCCCGATCTTC
AGAGCCACCATGAACTAAAACCACTGGAATGCTGTGAGTTTCCTTTTGGTTCCAAGCAGAAGGAGGTCTG
CATCAATCCCTACCACTATAAGAGAGTAGAAAGCCCTGTACTTCCTCCTGTGCTGGTTCCAAGACACAGC
GAATATAATCCTCAGCACAGCCTCTTAGCTCAGTTCCGTAACTTAGGACAAAATGAGCCTCACATGCCAC
TCAACGCCACTTTTCCAGATTCTTTCCAGCAACCCAACAGCCACCCGTTTCCTCACTCTCCCAATAGCAG
TTACCCAAACTCTCCTGGGAGCAGCAGCAGCACCTACCCTCACTCTCCCACCAGCTCAGACCCAGGAAGC
CCTTTCCAGATGCCAGCTGATACGCCCCCACCTGCTTACCTGCCTCCTGAAGACCCCATGACCCAGGATG
GCTCTCAGCCGATGGACACAAACATGATGGCGCCTCCCCTGCCCTCAGAAATCAACAGAGGAGATGTTCA
GGCGGTTGCTTATGAGGAACCAAAACACTGGTGCTCTATTGTCTACTATGAGCTCAACAATCGTGTGGGT
GAAGCGTTCCATGCCTCCTCCACAAGTGTGTTGGTGGATGGTTTCACTGATCCTTCCAACAATAAGAACC
GTTTCTGCCTTGGGCTGCTCTCCAATGTTAACCGGAATTCCACTATTGAAAACACCAGGCGGCATATTGG
AAAAGGAGTTCATCTTTATTATGTTGGAGGGGAGGTGTATGCCGAATGCCTTAGTGACAGTAGCATCTTT
GTGCAAAGTCGGAACTGCAACTACCATCATGGATTTCATCCTACTACTGTTTGCAAGATCCCTAGTGGGT
GTAGTCTGAAAATTTTTAACAACCAAGAATTTGCTCAGTTATTGGCACAGTCTGTGAACCATGGATTTGA
GACAGTCTATGAGCTTACAAAAATGTGTACTATACGTATGAGCTTTGTGAAGGGCTGGGGAGCAGAATAC
CACCGCCAGGATGTTACTAGCACCCCCTGCTGGATTGAGATACATCTGCACGGCCCCCTCCAGTGGCTGG
ATAAAGTTCTTACTCAAATGGGTTCACCTCATAATCCTATTTCATCTGTATCTTAAATGGCCCCAGGCAT
CTGCCTCTGGAAAACTATTGAGCCTTGCATGTACTTGAAGGATGGATGAGTCAGACACGATTGAGAACTG
ACAAAGGAGCCTTGATAATACTTGACCTCTGTGACCAACTGTTGGATTCAGAAATTTAAACAAAAAAAAA
AAAAAACACACACACCTTGGTAACATACTGTTGATATCAAGAACCTGTTTAGTTTACATTGTAACATTCT
ATTGTAAAATCAACTAAAATTCAGACTTTTAGCAGGACTTTGTGTACAGTTAAAGGAGAGATGGCCAAGC
CAGGGACAAATTGTCTATTAGAAAACGGTCCTAAGAGATTCTTTGGTGTTTGGCACTTTAAGGTCATCGT
TCTTTTCATGGGTTTTCATAATATTTTAAAACTATTTGTTTAGCGATGGTTTTGTTCGTTTAAGTAAAGG
TTAATCTTGATGATATACATAATAATCTTTCTAAAATTGTATGCTGACCATACTTGCTGTCAGAATAATG
CTAGGCATATGCTTTTTGCTAAATATGTATGTACAGAGTATTTGGAAGTTAAGAATTGATTAGACTAGTG
AATTTAGGAGTATTTGAGGTGGGTGGGGGGAAGAGGGAAATGACAACTGCAAATGTAGACTATACTGTAA
AAATTCAGTTTGTTGCTTTAAAGAAACAAACTGATACCTGAATTTTGCTGTGTTTCCATTTTTTAGAGAT
TTTTATCATTTTTTTCTCTCTCGGCATTCTTTTTTCTCATACTCTTCAAAAAGCAGTTCTGCAGCTGGTT
AATTCATGTAACTGTGAGAGCAAATGAATAATTCCTGCTATTCTGAAATTGCCTACATGTTTCAATACCA
GTTATATGGAGTGCTTGAATTTAATAAGCAGTTTTTACGGAGTTTACAGTACAGAAATAGGCTTTAATTT
TCAAGTGAATTTTTTGCCAAACTTAGTAACTCTGTTAAATATTTGGAGGATTTAAAGAACATCCCAGTTT
GAATTCATTTCAAACTTTTTAAATTTTTTTGTACTATGTTTGGTTTTATTTTCCTTCTGTTAATCTTTTG
TATTCACTTATGCTCTCGTACATTGAGTACTTTTATTCCAAAACTAGTGGGTTTTCTCTACTGGAAATTT
TCAATAAACCTGTCATTATTGCTTACTTTGATTAAAAA

### Polynucleotide/Nucleic Acid Molecules

Also provided herein are nucleic acid or polynucleotide molecules that encode the antibodies, antigen binding fragments thereof and/or polypeptides described herein. The nucleic acids may be present, for example, in whole cells, in a cell lysate, or in a partially purified or substantially pure form.

Nucleic acids described herein can be obtained using standard molecular biology techniques. For example, nucleic acid molecules described herein can be cloned using standard PCR techniques or chemically synthesized. For antibodies obtained from an immunoglobulin gene library (e.g., using phage or yeast display techniques), nucleic acid encoding the antibody or peptide can be recovered from the library.

Nucleic acids encoding any of the proteins described herein (e.g. β-catenin) are also provided herein. Such a nucleic acid may further be linked to a promoter and/or other regulatory sequences, as further described herein. Exemplary nucleic acids are those that are at least about 80%, 85%, 90%, 95%, 98%, 99% or 100% identical to a nucleotide sequence wildtype sequence of β-catenin, such as nucleic acid sequence encoding the protein fragments described herein. Nucleic acids may also hybridize specifically, e.g., under stringent hybridization conditions, to a nucleic acid described herein or a fragment thereof. Table 2 comprises exemplary β-catenin mRNA transcripts.

Nucleic acids, e.g., those encoding a protein described above, a functional homolog thereof, or a nucleic acid intended to inhibit the production of a protein of interest (e.g., siRNA, shRNA or antisense RNA, described in greater detail in this application) can be delivered to cells in culture, ex vivo, and in vivo. The delivery of nucleic acids can be by any technique known in the art including viral mediated gene transfer, liposome mediated gene transfer, direct injection into a target tissue, organ, or tumor, injection into vasculature which supplies a target tissue or organ. Exemplary mRNAs for GSK3 and SMAD1 can be found in Table 1.

Polynucleotides can be administered in any suitable formulations known in the art. These can be as virus particles, as naked DNA, in liposomes, in complexes with polymeric carriers, etc. Polynucleotides can be administered to the arteries which feed a tissue or tumor.

Nucleic acids can be delivered in any desired vector. These include viral or non-viral vectors, including adenovirus vectors, adeno-associated virus vectors, retrovirus vectors, lentivirus vectors, and plasmid vectors. Exemplary types of viruses include HSV (herpes simplex virus), AAV (adeno associated virus), HIV (human immunodeficiency virus), BIV (bovine immunodeficiency virus), and MLV (murine leukemia virus). Nucleic acids can be administered in any desired format that provides sufficiently efficient delivery levels, including in virus particles, in liposomes, in nanoparticles, and complexed to polymers.

A polynucleotide of interest can also be combined with a condensing agent to form a gene delivery vehicle. The condensing agent may be a polycation, such as polylysine, polyarginine, polyornithine, protamine, spermine, spermidine, and putrescine. Many suitable methods for making such linkages are known in the art.

In an alternative embodiment, a polynucleotide of interest is associated with a liposome to form a gene delivery vehicle. Liposomes are small, lipid vesicles comprised of an aqueous compartment enclosed by a lipid bilayer, typically spherical or slightly elongated structures several hundred Angstroms in diameter. Under appropriate conditions, a liposome can fuse with the plasma membrane of a cell or with the membrane of an endocytic vesicle within a cell which has internalized the liposome, thereby releasing its contents into the cytoplasm. Prior to interaction with the surface of a cell, however, the liposome membrane acts as a relatively impermeable barrier which sequesters and protects its contents, for example, from degradative enzymes. Additionally, because a liposome is a synthetic structure, specially designed liposomes can be produced which incorporate desirable features. See Stryer, Biochemistry, pp. 236-240, 1975 (W.H. Freeman, San Francisco, Calif.); Szoka et al., Biochim. Biophys. Acta 600:1, 1980; Bayer et al., Biochim. Biophys. Acta. 550:464, 1979; Rivnay et al., Meth. Enzymol. 149:119, 1987; Wang et al., PROC. NATL. ACAD. SCI. U.S.A. 84: 7851, 1987, Plant et al., Anal. Biochem. 176:420, 1989, and U.S. Pat. No. 4,762,915. Liposomes can encapsulate a variety of nucleic acid molecules including DNA, RNA, plasmids, and expression constructs comprising growth factor polynucleotides such those described herein

Liposomal preparations for use in the methods described herein include cationic (positively charged), anionic (negatively charged) and neutral preparations. Cationic liposomes have been shown to mediate intracellular delivery of plasmid DNA (Felgner et al., Proc. Natl. Acad. Sci. USA 84:7413-7416, 1987), mRNA (Malone et al., Proc. Natl. Acad. Sci. USA 86:6077-6081, 1989), and purified transcription factors (Debs et al., J. Biol. Chem. 265:10189-10192, 1990), in functional form. Cationic liposomes are readily available. For example, N[1-2,3-dioleyloxy)propyl]-N,N,N-triethylammonium (DOTMA) liposomes are available under the trademark Lipofectin, from GIBCO BRL, Grand Island, N.Y. See also Felgner et al., Proc. Natl. Acad. Sci. USA 91: 5148-5152.87, 1994. Other commercially available liposomes include Transfectace (DDAB/DOPE) and DOTAP/DOPE (Boerhinger). Other cationic liposomes can be prepared from readily available materials using techniques well known in the art. See, e.g., Szoka et al., Proc. Natl. Acad. Sci. USA 75:4194-4198, 1978; and WO 90/11092 for descriptions of the synthesis of DOTAP (1,2-bis(oleoyloxy)-3-(trimethylammonio)propane) liposomes.

Similarly, anionic and neutral liposomes are readily available, such as from Avanti Polar Lipids (Birmingham, Ala.), or can be easily prepared using readily available materials. Such materials include phosphatidyl choline, cholesterol, phosphatidyl ethanolamine, dioleoylphosphatidyl choline (DOPC), dioleoylphosphatidyl glycerol (DOPG), dioleoylphoshatidyl ethanolamine (DOPE), among others. These materials can also be mixed with the DOTMA and DOTAP starting materials in appropriate ratios. Methods for making liposomes using these materials are well known in the art.

TABLE 2
H.sapiens mRNA for beta-catenin (SEQ ID NO: 12)

AAGCCTCTCGGTCTGTGGCAGCAGCGTTGGCCCGGCCCCGGGAGCGGAGAGCGAGGGGAGGCGGAGACGG
AGGAAGGTCTGAGGAGCAGCTTCAGTCCCCGCCGAGCCGCCACCGCAGGTCGAGGACGGTCGGACTCCCG
CGGCGGGAGGAGCCTGTTCCCCTGAGGGTATTTGAAGTATACCATACAACTGTTTTGAAAATCCAGCGTG
GACAATGGCTACTCAAGCTGATTTGATGGAGTTGGACATGGCCATGGAACCAGACAGAAAAGCGGCTGTT
AGTCACTGGCAGCAACAGTCTTACCTGGACTCTGGAATCCATTCTGGTGCCACTACCACAGCTCCTTCTC
TGAGTGGTAAAGGCAATCCTGAGGAAGAGGATGTGGATACCTCCCAAGTCCTGTATGAGTGGGAACAGGG
ATTTTCTCAGTCCTTCACTCAAGAACAAGTAGCTGATATTGATGGACAGTATGCAATGACTCGAGCTCAG
AGGGTACGAGCTGCTATGTTCCCTGAGACATTAGATGAGGGCATGCAGATCCCATCTACACAGTTTGATG
CTGCTCATCCCACTAATGTCCAGCGTTTGGCTGAACCATCACAGATGCTGAAACATGCAGTIGTAAACTT
GATTAACTATCAAGATGATGCAGAACTTGCCACACGTGCAATCCCTGAACTGACAAAACTGCTAAATGAC
GAGGACCAGGTGGTGGTTAATAAGGCTGCAGTTATGGTCCATCAGCTTTCTAAAAAGGAAGCTTCCAGAC
ACGCTATCATGCGTTCTCCTCAGATGGTGTCTGCTATTGTACGTACCATGCAGAATACAAATGATGTAGA
AACAGCTCGTTGTACCGCTGGGACCTTGCATAACCTTTCCCATCATCGTGAGGGCTTACTGGCCATCTTT
AAGTCTGGAGGCATTCCTGCCCTGGTGAAAATGCTTGGTTCACCAGTGGATTCTGTGTTGTTTTATGCCA
TTACAACTCTCCACAACCTTTTATTACATCAAGAAGGAGCTAAAATGGCAGTGCGTTTAGCTGGTGGGCT
GCAGAAAATGGTTGCCTTGCTCAACAAAACAAATGTTAAATTCTTGGCTATTACGACAGACTGCCTTCAA
ATTTTAGCTTATGGCAACCAAGAAAGCAAGCTCATCATACTGGCTAGTGGTGGACCCCAAGCTTTAGTAA
ATATAATGAGGACCTATACTTACGAAAAACTACTGTGGACCACAAGCAGAGTGCTGAAGGTGCTATCTGT
CTGCTCTAGTAATAAGCCGGCTATTGTAGAAGCTGGTGGAATGCAAGCTTTAGGACTTCACCTGACAGAT
CCAAGTCAACGTCTTGTTCAGAACTGTCTTTGGACTCTCAGGAATCTTTCAGATGCTGCAACTAAACAGG
AAGGGATGGAAGGTCTCCTTGGGACTCTTGTTCAGCTTCTGGGTTCAGATGATATAAATGTGGTCACCTG
TGCAGCTGGAATTCTTTCTAACCTCACTTGCAATAATTATAAGAACAAGATGATGGTCTGCCAAGTGGGT
GGTATAGAGGCTCTTGTGCGTACTGTCCTTCGGGCTGGTGACAGGGAAGACATCACTGAGCCTGCCATCT
GTGCTCTTCGTCATCTGACCAGCCGACACCAAGAAGCAGAGATGGCCCAGAATGCAGTTCGCCTTCACTA
TGGACTACCAGTTGTGGTTAAGCTCTTACACCCACCATCCCACTGGCCTCTGATAAAGGCTACTGTTGGA
TTGATTCGAAATCTTGCCCTTTGTCCCGCAAATCATGCACCTTTGCGTGAGCAGGGTGCCATTCCACGAC
TAGTTCAGTIGCTTGTTCGTGCACATCAGGATACCCAGCGCCGTACGTCCATGGGTGGGACACAGCAGCA
ATTTGTGGAGGGGGTCCGCATGGAAGAAATAGTTGAAGGTTGTACCGGAGCCCTTCACATCCTAGCTCGG
GATGTTCACAACCGAATTGTTATCAGAGGACTAAATACCATTCCATTGTTTGTGCAGCTGCTTTATTCTC
CCATTGAAAACATCCAAAGAGTAGCTGCAGGGGTCCTCTGTGAACTTGCTCAGGACAAGGAAGCTGCAGA
AGCTATTGAAGCTGAGGGAGCCACAGCTCCTCTGACAGAGTTACTTCACTCTAGGAATGAAGGIGTGGCG
ACATATGCAGCTGCTGTTTTGTTCCGAATGTCTGAGGACAAGCCACAAGATTACAAGAAACGGCTTTCAG
TTGAGCTGACCAGCTCTCTCTTCAGAACAGAGCCAATGGCTTGGAATGAGACTGCTGATCTTGGACTTGA
TATTGGTGCCCAGGGAGAACCCCTTGGATATCGCCAGGATGATCCTAGCTATCGTTCTTTTCACTCTGGT
GGATATGGCCAGGATGCCTTGGGTATGGACCCCATGATGGAACATGAGATGGGTGGCCACCACCCTGGTG
CTGACTATCCAGTTGATGGGCTGCCAGATCTGGGGCATGCCCAGGACCTCATGGATGGGCTGCCTCCAGG
TGACAGCAATCAGCTGGCCTGGTTTGATACTGACCTGTAAATCATCCTTTAGCTGTATTGTCTGAACTTG
CATTGTGATTGGCCTGTAGAGTTGCTGAGAGGGCTCGAGGGGTGGGCTGGTATCTCAGAAAGTGCCTGAC
ACACTAACCAAGCTGAGTTTCCTATGGGAACAATTGAAGTAAACTTTTTGTTCTGGTCCTTTTTGGTCGA
GGAGTAACAATACAAATGGATTTTGGGAGTGACTCAAGAAGTGAAGAATGCACAAGAATGGATCACAAGA
TGGAATTTAGCAAACCCTAGCCTTGCTTGTTAAAATTTTTTTTTTTTTTTTTTTAAGAATATCTGTAATG
GTACTGACTTTGCTTGCTTTGAAGTAGCTCTTTTTTTTTTTTTTTTTTTTTTTTTTTGCAGTAACTGITT
TTTAAGTCTCTCGTAGTGTTAAGTTATAGTGAATACTGCTACAGCAATTTCTAATTTTTAAGAATTGAGT
AATGGTGTAGAACACTAATTAATTCATAATCACTCTAATTAATTGTAATCTGAATAAAGTGTAACAATTG
TGTAGCCTTTTTGTATAAAATAGACAAATAGAAAATGGTCCAATTAGTTTCCTTTTTAATATGCTTAAAA
TAAGCAGGTGGATCTATTTCATGTTTTTGATCAAAAACTATTTGGGATATGTATGGGTAGGGTAAATCAG
CTGCTGTGATACGATGCTTCAAGAGAAAATGCGGTTATAAAAAATGGTTCAGAATTAAACTTTTAATTCA
TT

### Polypeptides

In certain embodiments, provided herein is isolated polypeptides capable of modulating the activity of GSK3 (e.g., GSK33), SMAD1, and or β-catenin. Such polypeptides can be useful, for example, for inhibiting the activity of GSK3 (e.g., GSK33) and/or SMAD1, or activating β-catenin and for identifying and/or generating agents that specifically bind to GSK3 (e.g., GSK33), SMAD1, or β-catenin. In some embodiments, the agonist is a transcriptional co-activator of β-catenin. The CREB binding protein (CBP) and the closely related protein p300 can assemble with β-catenin and act as β-catenin binding transcriptional coactivators. For example, to generate a transcriptionally active complex, β-catenin recruits the transcriptional coactivators, CREB-binding protein (CBP) or its closely related homolog p300 (Hecht et al., EMBO J. 19:1839-50 (2000); Takemaru et al., J.2020200825 5 Feb. 2020 Cell Biol. 149:249-54 (2000)) as well as other components of the basal transcription machinery. Additional β-catenin co-activators include TBP, BRG1, and BCL9/PYG. Exemplary β-catenin pathway agonists act on one or more components of the β-catenin signaling pathway to thereby express or increase activity or levels of β-catenin. For example, suitable β-catenin pathway agonists can enhance β-catenin stability. Agents may act by reducing and/or by promoting the release of sequestered endogenous intracellular β-catenin. Exemplary β-catenin pathway agonists include, but are not limited to, for example, Wnt ligand, DSH/DVL1, 2, 3, LRP6ΔN, WNT3A, WNT5A, and WNT3A. Additional β-catenin pathway activators are reviewed in the art (Moon et al., Nature Reviews Genetics, 5: 689-699, 2004, hereby incorporated by reference in its entirety). In some embodiments, suitable β-catenin pathway agonists can include antibodies and antigen-binding fragments and peptides that specifically bind to the frizzled (Fzd) family of receptors.

In some embodiments, the polypeptides can be isolated from cells or tissue sources by an appropriate purification scheme using standard protein purification techniques. In another embodiment, polypeptides are produced by recombinant DNA techniques. Alternatively, polypeptides can be chemically synthesized using standard peptide synthesis techniques. In some embodiments, the test agent is a chimeric or fusion polypeptide. A fusion or chimeric polypeptide can be produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, Ausubel et al., eds., John Wiley & Sons: 1992). Moreover, many expression vectors are commercially available that already encode a fusion moiety.

The polypeptides described herein can be produced in prokaryotic or eukaryotic host cells by expression of polynucleotides encoding a polypeptide(s). Alternatively, such peptides can be synthesized by chemical methods. Methods for expression of heterologous polypeptides in recombinant hosts, chemical synthesis of polypeptides, and in vitro translation are well known in the art and are described further in Maniatis et al., Molecular Cloning: A Laboratory Manual (1989), 2nd Ed., Cold Spring Harbor, N.Y.; Berger and Kimmel, Methods in Enzymology, Volume 152, Guide to Molecular Cloning Techniques (1987), Academic Press, Inc., San Diego, Calif.; Merrifield, J. (1969) J. Am. Chem. Soc. 91:501; Chaiken I. M. (1981) CRC Crit. Rev. Biochem. 11:255; Kaiser et al. (1989) Science 243:187; Merrifield, B. (1986) Science 232:342; Kent, S. B. H. (1988) Annu. Rev. Biochem. 57:957; and Offord, R. E. (1980) Semisynthetic Proteins, Wiley Publishing, which are incorporated herein by reference.

### CRISPR/Gene Editing

Certain embodiments disclosed herein relate to agents and methods for treating or preventing a condition (e.g., any condition, disease, disorder, or indication disclosed herein) in a subject comprising administering an agent (e.g., a gene editing agent) that edits a gene encoding GSK (e.g. GSKbeta or SMAD1).

In some embodiments, the agent disclosed herein is an agent for genome editing (e.g., an agent used to delete at least a portion of a gene that encodes a GSK or SMAD1 protein). Deletion of DNA may be performed using gene therapy to knock-out or disrupt the target gene. As used herein, a “knock-out” can be a gene knock-down or the gene can be knocked out by a mutation such as, a point mutation, an insertion, a deletion, a frameshift, or a missense mutation by techniques known in the art, including, but not limited to, retroviral gene transfer. In some embodiments, the agent is a nuclease (e.g., a zinc finger nuclease or a TALEN). Zinc-finger nucleases (ZFNs) are artificial restriction enzymes generated by fusing a zinc finger DNA-binding domain to a DNA-cleavage domain. Zinc finger domains can be engineered to target desired DNA sequences, which enable zinc-finger nucleases to target unique sequence within a complex genome. By taking advantage of endogenous DNA repair machinery, these reagents can be used to precisely alter the genomes of higher organisms. Other technologies for genome customization that can be used to knock out genes are meganucleases and TAL effector nucleases (TALENs). A TALEN is composed of a TALE DNA binding domain for sequence-specific recognition fused to the catalytic domain of an endonuclease that introduces double-strand breaks (DSB). The DNA binding domain of a TALEN is capable of targeting with high precision a large recognition site (for instance, 17 bp). Meganucleases are sequence-specific endonucleases, naturally occurring “DNA scissors,” originating from a variety of single-celled organisms such as bacteria, yeast, algae and some plant organelles. Meganucleases have long recognition sites of between 12 and 30 base pairs. The recognition site of natural meganucleases can be modified in order to target native genomic DNA sequences (such as endogenous genes).

In another embodiment, the agent comprises a CRISPR-Cas9 guided nuclease and/or a sgRNA (Wiedenheft et al., “RNA-Guided Genetic Silencing Systems in Bacteria and Archaea,” Nature 482:331-338 (2012); Zhang et al., “Multiplex Genome Engineering Using CRISPR/Cas Systems,” Science 339(6121): 819-23 (2013); and Gaj et al., “ZFN, TALEN, and CRISPR/Cas-based Methods for Genome Engineering,” Cell 31(7):397-405 (2013), which are hereby incorporated by reference in their entirety). Like the TALENs and ZFNs, CRISPR-Cas9 interference is a genetic technique which allows for sequence-specific control of gene expression in prokaryotic and eukaryotic cells by guided nuclease double-stranded DNA cleavage. It is based on the bacterial immune system-derived CRISPR (clustered regularly interspaced palindromic repeats) pathway. In some embodiments, the agent is an sgRNA. An sgRNA combines tracrRNA and crRNA, which are separate molecules in the native CRISPR/Cas9 system, into a single RNA construct, simplifying the components needed to use CRISPR/Cas9 for genome editing. In some embodiments, the crRNA of the sgRNA has complementarity to at least a portion of a gene that encodes GSK or SMAD1 (or a fragment thereof). In some embodiments, the sgRNA may target at least a portion of a gene that encodes a GSK or SMAD1 protein.

### Methods of Identifying Modulators of GSK3, SMAD1 or β-Catenin Activity

Provided herein are methods and compositions for determining whether a test agent is an inhibitor of vascular calcification and/or osteogenesis.

The methods may comprise forming a test mixture comprising: a test agent, wherein the test agent is a polynucleotide, a small molecule, or a peptide and contacting the test mixture with cells expressing GSK3 (e.g., GSK33); wherein a test agent that inhibits the activity of or decreases the levels of GSK3 compared to the activity of or level of GSK3 in a control mixture is an inhibitor of vascular calcification and/or osteogenesis.

The methods may comprise forming a test mixture comprising: a test agent, wherein the test agent is a polynucleotide, a small molecule, or a peptide and contacting the test mixture with cells expressing SMAD1; wherein a test agent that inhibits the activity of or decreases the levels of SMAD1 compared to the activity of or level of SMAD1 in a control mixture is an inhibitor of vascular calcification and/or osteogenesis.

The methods may comprise forming a test mixture comprising: a test agent, wherein the test agent is a polynucleotide, a small molecule, or a peptide and contacting the test mixture with cells expressing β-catenin; wherein a test agent that increases the activity of β-catenin compared to the activity of or level of β-catenin in a control mixture is an inhibitor of vascular calcification and/or osteogenesis.

Provided herein are methods and compositions for determining whether a test agent is an potentiator or activator of osteoblastic-endothelial transdifferentiation. The methods may comprise forming a test mixture comprising: a test agent, wherein the test agent is a polynucleotide, a small molecule, or a peptide and contacting the test mixture with cells expressing GSK3 (e.g., GSK33); wherein a test agent that inhibits the activity of or decreases the levels of GSK3 compared to the activity of or level of GSK3 in a control mixture is an potentiator or activator of osteoblastic-endothelial transdifferentiation.

The methods may comprise forming a test mixture comprising: a test agent, wherein the test agent is a polynucleotide, a small molecule, or a peptide and contacting the test mixture with cells expressing SMAD1; wherein a test agent that inhibits the activity of or decreases the levels of SMAD1 compared to the activity of or level of SMAD1 in a control mixture is an potentiator or activator of osteoblastic-endothelial transdifferentiation.

The methods may comprise forming a test mixture comprising: a test agent, wherein the test agent is a polynucleotide, a small molecule, or a peptide and contacting the test mixture with cells expressing β-catenin; wherein a test agent that increases the activity of β-catenin compared to the activity of or level of β-catenin in a control mixture is an potentiator or activator of osteoblastic-endothelial transdifferentiation.

The test agent may be linked to a detectable moiety. As used herein, a detectable moiety may comprise a test agent or other peptide of the present invention linked to a distinct polypeptide or moiety to which it is not linked in nature. For example, the detectable moiety can be fused to the N-terminus or C-terminus of the test agent either directly, through a peptide bond, or indirectly through a chemical linker.

The GSK3, SMAD1, or β-catenin may be linked to a detectable moiety. The test agent may be a peptide, small molecule, or a polynucleotide (e.g., an inhibitory polynucleotide or a nucleotide encoding β-catenin).

In some embodiments, the control mixture is substantially identical to the test mixture except that the control mixture does not comprise a test agent. In some embodiments, the test agent is a member of a library of test agents.

Alternatively, agents may be screened for and identified as agents useful in the present application by detecting osteogenic markers and/or endothelial markers. Specifically, a reduction of osteogenic markers (e.g., Cbfa1, osterix and osteocalcin) and/or the induction of endothelial markers (e.g., CD34, VE-cadherin, CD31 and eNOS) once an agent is contacted to cells can show that the agent is useful in inhibiting vascular calcification and/or osteogenesis or increasing osteoblastic-endothelial transdifferentiation.

Agents useful in the methods of the present invention may be obtained from any available source, including systematic libraries of natural and/or synthetic compounds. Agents may also be obtained by any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann et al., 1994, J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, 1997, Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of agents may be presented in solution (e.g., Houghten, 1992, Biotechniques 13:412-421), or on beads (Lam, 1991, Nature 354:82-84), chips (Fodor, 1993, Nature 364:555-556), bacteria and/or spores, (Ladner, U.S. Pat. No. 5,223,409), plasmids (Cull et al, 1992, Proc Natl Acad Sci USA 89:1865-1869) or on phage (Scott and Smith, 1990, Science 249:386-390; Devlin, 1990, Science 249:404-406; Cwirla et al, 1990, Proc. Natl. Acad. Sci. 87:6378-6382; Felici, 1991, J. Mol. Biol. 222:301-310; Ladner, supra.).

### Pharmaceutical Compositions

In certain embodiments, provided herein is a composition, e.g., a pharmaceutical composition, containing at least one agent described herein together with a pharmaceutically acceptable carrier. In one embodiment, the composition includes a combination of multiple (e.g., two or more) agents described herein.

As described in detail below, the pharmaceutical compositions disclosed herein may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; or (2) parenteral administration, for example, by subcutaneous, intramuscular, intravenous, intrathecal, intracerebral or epidural injection as, for example, a sterile solution or suspension, or sustained-release formulation.

Methods of preparing these formulations or compositions include the step of bringing into association an agent described herein with the carrier and, optionally, one or more accessory ingredients. In general, the formulations are prepared by uniformly and intimately bringing into association an agent described herein with liquid carriers, or finely divided solid carriers, or both, and then, if necessary, shaping the product.

Pharmaceutical compositions suitable for parenteral administration comprise one or more agents described herein in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions include water, ethanol, dimethyl sulfoxide (DMSO), polyols (such as glycerol, propylene glycol, polyethylene glycol, and the like), and suitable mixtures thereof, vegetable oils, such as olive oil, and injectable organic esters, such as ethyl oleate.

Proper fluidity can be maintained, for example, by the use of coating materials, such as lecithin, by the maintenance of the required particle size in the case of dispersions, and by the use of surfactants.

Regardless of the route of administration selected, the agents provided herein, which may be used in a suitable hydrated form, and/or the pharmaceutical compositions disclosed herein, are formulated into pharmaceutically-acceptable dosage forms by conventional methods known to those of skill in the art.

### Methods

Disclosed herein are novel therapeutic methods of treatment or prevention of diseases and/or disorders associated with vascular calcification, osteoblastic-endothelial transdifferentiation, and/or osteogenesis. Such diseases include but are not limited to cardiovascular disease, chronic kidney disease, diabetes mellitus, or fibrodysplasia ossificans progressiva.

Provided herein are methods of treating or preventing vascular calcification in a subject in need thereof comprising administering to the subject one or more test agent(s) identified by any one of the methods discussed herein. Also provided herein are methods of inducing or increasing osteoblastic-endothelial transdifferentiation in a subject in need thereof, comprising administering to the subject one or more test agents identified by any one of the methods discussed herein.

In some aspects, provided herein are methods of decreasing or inhibiting osteogeneisis in a subject in need thereof, comprising administering to the subject one or more test agent(s) identified using the methods disclosed herein.

The agent and/or pharmaceutical compositions disclosed herein may be delivered by any suitable route of administration, including orally, nasally, as by, for example, a spray, rectally, intravaginal, parenterally, intracisternally and topically, as by powders, ointments or drops, including buccally and sublingually. In certain embodiments the pharmaceutical compositions are delivered generally (e.g., via oral or parenteral administration). In certain other embodiments the pharmaceutical compositions are delivered locally through injection.

The methods disclosed herein include administration of one or more (e.g., two or more, three or more, or four or more) agents to the subject. The therapeutic described herein may be administered through conjunctive therapy. Conjunctive therapy includes sequential, simultaneous and separate, and/or co-administration of the active compounds in a such a way that the therapeutic effects of the first agent administered have not entirely disappeared when the subsequent agent is administered. In certain embodiments, the second agent may be co-formulated with the first agent or be formulated in a separate pharmaceutical composition.

In certain embodiments, provided herein are therapeutic methods of treating cardiovascular disease, chronic kidney disease, diabetes mellitus, or fibrodysplasia ossificans progressiva, comprising administering to a subject, (e.g., a subject in need thereof), an effective amount of an agent described herein. A subject in need thereof may include, for example, a subject who has been diagnosed with a disease or disorder disclosed herein, a subject predisposed to a disease or disorder disclosed herein, or a subject who has been treated for a disease or disorder disclosed herein, including subjects that have been refractory to the previous treatment.

Actual dosage levels of the active ingredients in the pharmaceutical compositions may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular agent employed, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could prescribe and/or administer doses of the compounds employed in the pharmaceutical composition at levels lower than that required in order to achieve the desired therapeutic effect and gradually increase the dosage until the desired effect is achieved.

### EXEMPLIFICATION

Transitions between cell fates commonly occur in development and disease. However, shifting unwanted cell fate back to normal differentiation in order to treat disease remains an unexplored area. The data provided herein demonstrate a process to transit ill-fated cells toward normalization in vascular calcification. Vascular endothelium is known to contribute osteoprogenitors to calcification through endothelial-mesenchymal transitions, in which endothelial cells gain plasticity and differentiate into osteoblast-like cells. Provided herein is a high throughput screen which identified SB216763, an inhibitor of glycogen synthase kinase 3 (GSK3), to convert osteoblasts into endothelial-like cells. It is demonstrated that SB216763 prevents endothelial-derived osteogenic differentiation at an early stage of vascular calcification, and shows that SB216763 converts osteoblasts into endothelial-like cells to reduce late-stage calcification, where osteoblastic-lineage tracing concludes that SB216763 shifts osteoblast to endothelial differentiation. Deletion of GSK3β in osteoblasts recapitulates osteoblastic-endothelial transdifferentiation and reduces vascular calcification. In addition, SB216763 treatment has no effect on bone formation. Provided herein are methods and compositions to accomplish a switch of ill-fated cells toward normalization, and GSK3β inhibition provides a new strategy for halting the progression of vascular calcification.

### Results

### High Throughput Screen Identified GSK3 Inhibitor SB216763 to Induce Osteoblastic-Endothelial Transdifferentiation

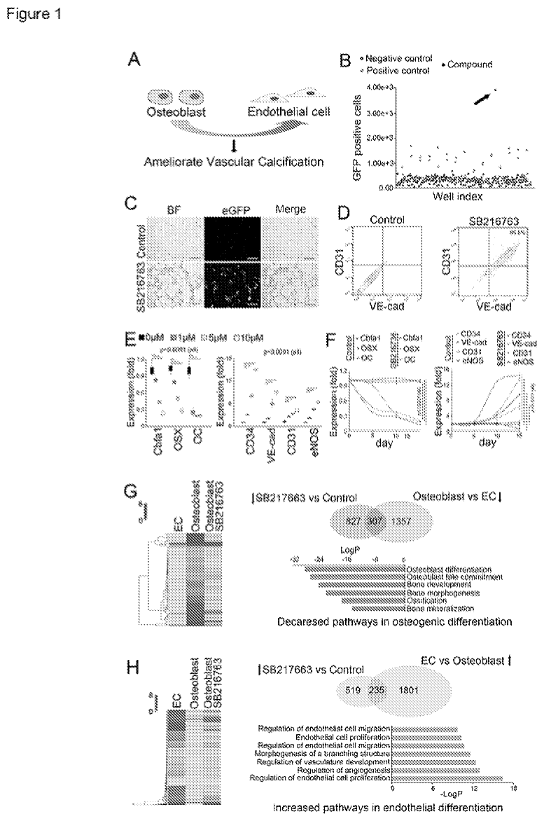

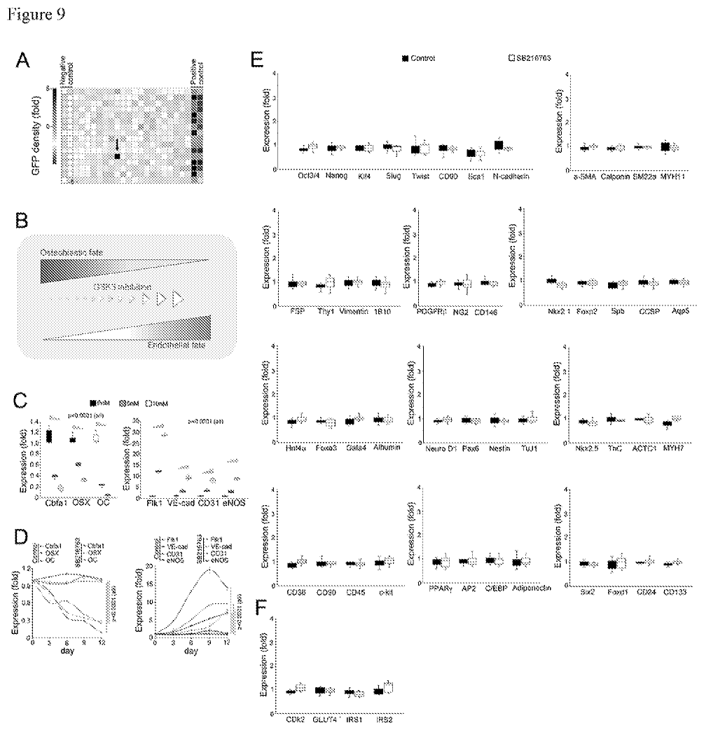

A high throughput screen was generated by modifying the mouse osteoblast line MC3T3. Fetal liver kinase 1 (Flk1) promoter-driven enhanced green fluorescent protein (eGFP) was introduced into these osteoblasts. Expression of eGFP indicated that the cells were able to undergo osteoblastic-endothelial transdifferentiation. Several libraries of small molecules, including a FDA-approved drug library, a UCLA in-house collection, and a custom set of compounds were screened. These libraries contained more than 22,000 small molecules ranging from natural products to synthesized compounds. After 14 days of treatment, high throughput screen identified GSK3 inhibitor SB216763 (FIG. 1, Part B, FIG. 9, Part A), which strongly activated eGFP expression and converted osteoblasts into EC-like morphology (FIG. 1, Part C). Flow cytometric analysis showed that more than 85% SB216763-treated osteoblasts expressed the endothelial markers CD31 and VE-cadherin (FIG. 1, Part D). Using arrange of dose (1-10 μM), it is shown that the reduction of the osteogenic markers Cbfa1, osterix and osteocalcin and the induction of the endothelial markers CD34, VE-cadherin, CD31 and eNOS occurred in a dose-dependent manner (FIG. 1, Part E). Osteoblasts were then treated with 10 μM SB216763 and examined the time-course expression of these osteogenic and endothelial markers. The results showed that a reduction of osteogenic markers preceded the induction of the endothelial markers (FIG. 1, Part F), suggesting that the osteoblasts lost their cell fate before undergoing endothelial differentiation (FIG. 9, Part B). This finding was confirmed in the human osteoblasts line hFOB 1.19, where the expression patterns of these markers were similar to the mouse osteoblasts (FIG. 9, Parts C and D), suggesting that the switch of osteoblastic fate to endothelial differentiation by GSK3 inhibition is similar in human and mouse cells. To determine if a mesenchymal stage or emerging stemness is required for SB216763 to induce osteoblastic-endothelial transdifferentiation, mesenchymal and stem cell markers were examined and found no induction in the SB216763-treated cells (FIG. 9, Part E). It was further determined the specificity of endothelial differentiation by closely examining other vascular lineage markers including smooth muscle cells (SMCs), pericytes, and fibroblasts by using real-time PCR. In addition, no-vascular lineages, such as cardiac, neuronal, hepatic, pulmonary, renal, hematopoietic and adipogenic lineages were examined. No changes were found in these markers (FIG. 9, Part E). Because GSK3 is important in glucose metabolism, the expression of genes related to glucose metabolism was determined in the SB216763-treated osteoblasts, such as casein kinase 2, glucose transporter 4, and insulin receptor substrates 1 and 2. No changes were detected (FIG. 9, Part F).

The differential expression profiles of SB216763-treated osteoblasts using RNA sequencing was examined. The results showed that SB216763 altered the transcript profile of osteoblasts and increased the similarities between the treated osteoblasts and ECs (FIG. 1, Parts G and H). Using a 4-fold differential threshold level, it was found that 827 genes decreased in expression in SB21673-treated osteoblasts (FIG. 1, Part G). These overlapped with 1357 genes identified as low expression when osteoblast vs EC was compared, and a 307-gene cohort with decreased expression in both analyses was identified (FIG. 1, Part G). Gene Ontology (GO) termed these genes enriched for the signal pathway of osteoblastic differentiation and bone development (FIG. 1, Part G). Similarly, 519 genes with increased expression after SB21673 treatment were found (FIG. 1, Part H). These overlapped with 1801 genes with high expression when EC vs osteoblast was compared, and another cohort of 235 genes that showed increased expression in both analyses was identified (FIG. 1, Part H). GO term revealed these genes to be enriched in the signal pathway of endothelial differentiation and vascular formation (FIG. 1, Part H).

### SB216763 Rendered Osteoblasts to Lose the Capacity for Bone Formation but Gain Endothelial Function

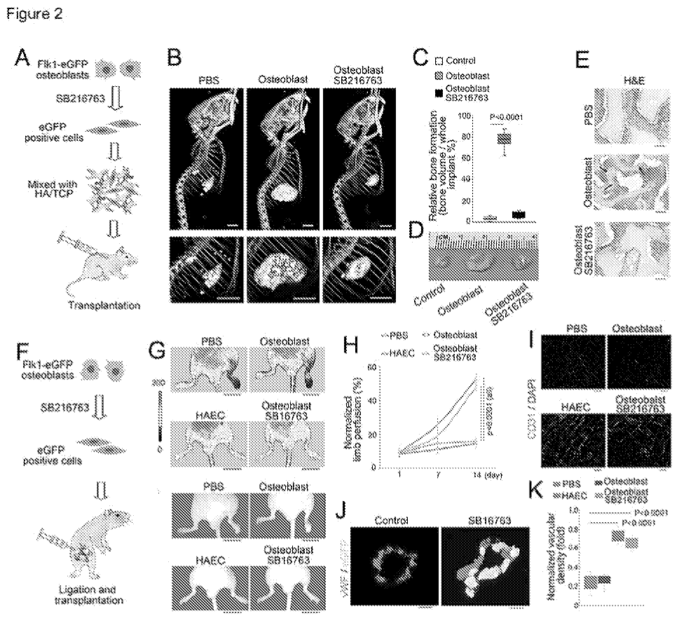

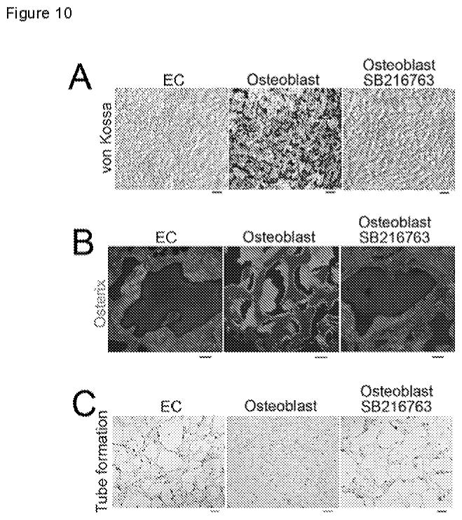

To assess the functional capacity of SB216763-treated osteoblasts, they were treated with osteogenic induction media in vitro. Von Kossa staining showed a lack of mineralization in the SB216763-treated cells (FIG. 10, Part A). A transplantation experiment was then performed to examine the capacity for bone formation using a well-established bone formation assay in vivo. This assay was adapted and eGFP positive cells were used, which were isolated from SB216763-treated Flk1-eGFP osteoblasts (FIG. 2, Part A). Micro-computed tomography (microCT) showed smaller size of ectopic bone, less trabecular formation and less bone volume in the implants with eGFP positive cells than the osteoblast controls (FIG. 2, Part B-D). Histology and immunostaining both confirmed a lack of osteocytes in the implants with the eGFP positive cells (FIG. 2, Part E, FIG. 10, Part B).

eGFP positive cells in tube formation assays in vitro were also tested, which showed a robust tube formation (FIG. 10, Part C). Then, same cells in a model of hindlimb ischemia in nude mice to evaluate the capacity of eGFP positive cells in vascular repair were tested. After ligation of the proximal and distal femoral artery, eGFP positive cells were transplanted (FIG. 2, Part F). Human aortic endothelial cells (HAECs) and osteoblasts were used as controls. Laser Doppler perfusion imaging demonstrated significantly higher limb blood flow in the mice with transplanted eGFP positive cells compared to osteoblasts (FIG. 2, Part G-H). Histology and immunostaining showed that the eGFP positive cells directly contributed to an increase in vascular density (FIG. 2, Part I-K), altogether suggesting that SB216763-treated cells gained endothelial functional in vascular reparation.

Alteration of SMAD1 and β-Catenin were Responsible for Osteoblastic-Endothelial Transdifferentiation

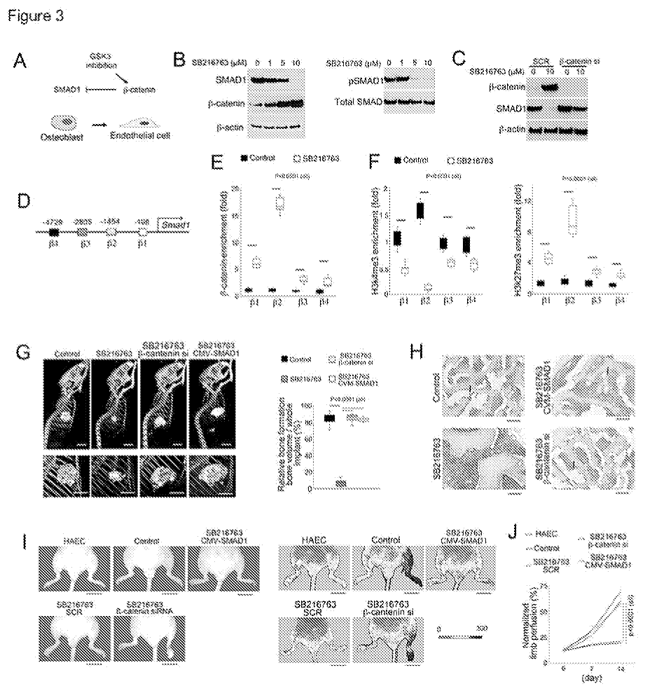

The expression of transcription factors that are known to be involved in bone formation or vascular development were screened. A dose-dependent decrease of expression and phosphorylation of SMAD1 with a robust increase of β-catenin in SB216763-treated osteoblasts was determined by immunoblotting, and it was hypothesized that the protein levels of SMAD1 and β-catenin were modulated by GSK3 inhibition and constituted key factors in the osteoblastic-endothelial transdifferentiation (FIG. 3, Parts A-B). GSK3-mediated phosphorylation of β-catenin directly caused the destabilization and degradation and the increase of β-catenin in SB216763-treated osteoblasts just confirmed these studies. To determine if excess β-catenin affected SMAD1 expression, β-catenin in SB216763-treated osteoblasts was depleted by using siRNA. The result showed that the depletion of β-catenin prevented decrease of SMAD1 (FIG. 3, Part C), suggesting that SMAD1 was downstream targeted by β-catenin for suppression. DNA-binding sites of β-catenin by locating the binding motifs in Smad1 gene was explored, and identified four individual sites located at −196 bp, −1454 bp, −2805 bp and −4729 bp upstream of exon1 of Smad1 gene (FIG. 3, Part D). ChIP assay confirmed more abundance of β-catenin around these sites in SB216763-treated osteoblasts than non-treated controls (FIG. 3, Part E). Then, transcriptional status of Smad1 was determined by examining the histone modification, including trimethylated histone H3 lysine 4 (H3K4me3) associated with active transcription and H3K27me3 a closed chromatin mark. The results showed lower abundance of H3K4me3 with higher abundance H3K27me3 around the same DNA-binding sites of β-catenin in the promoter region of Smad1 gene (FIG. 3, Part F). Together, the results suggested that the increase of β-catenin directly targeted transcriptional regulation of Smad1 and suppressed its expression.

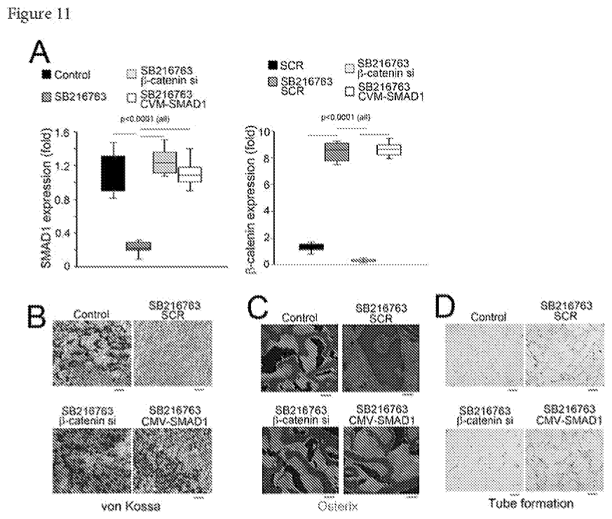

SMAD1 in SB216763-treated osteoblasts using lentiviral vectors containing CMV promotor-driven SMAD1 cDNA or depleted β-catenin using lentiviral vectors containing β-catenin-specific siRNA was explored (FIG. 11, Part A). β-catenin-specific siRNA efficiently lowered down β-catenin also prevented decrease of SMAD1, while restored SMAD1 had no effects on β-catenin level, again showed β-catenin is an upstream regulator of SMAD1 expression (FIG. 11, Part A). The cells were then subjected to osteogenic induction media. Von Kossa staining showed clear mineralization after restoring SMAD1 or depletion of β-catenin in the SB216763-treated cells (FIG. 11, Part B). Cells was transplanted into nude mice to test ectopic bone formation. MicroCT showed similar size of ectopic bone and bone volume between osteoblast controls and SB216763-treated cells with SMAD1 overexpression or knockdown of β-catenin (FIG. 3G). Histology and immunostaining confirmed the presence of osteocytes in these implants (FIG. 3H, FIG. 11, Part C). The results suggested that restoring SMAD1 or knockdown of β-catenin in SB216763-treated osteoblasts prevented the loss of the capacity of bone formation.

Tube formation assays in vitro was then tested. The results showed a decrease of tube formation only in SB216763-treated cells with β-catenin depletion (FIG. 11, Part D). Cells into hindlimb ischemia mouse model were transplanted. Laser Doppler perfusion imaging showed less blood flow in the mice transplanted with SB216763-treated osteoblasts with depletion of β-catenin than cells without depletion (FIG. 3, Parts I-J). No difference was observed between cells with restoring SMAD1 and controls (FIG. 3, Parts I-J). The results suggested that depletion of β-catenin decreased the SB216763-induced the capacity of vascular repair.

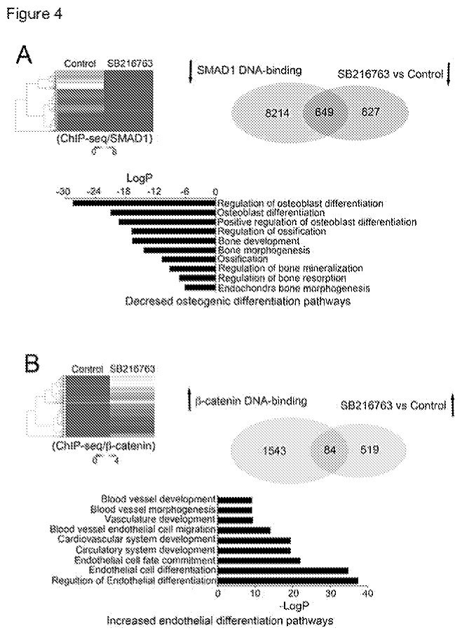

A chromatin immunoprecipitation was performed with massively parallel DNA sequencing (ChIP-seq) to examine potential alterations of SMAD1 or β-catenin DNA-binding in SB216763-treated osteoblasts. Homer tool detected significant alterations of the SMAD1 and β-catenin enrichment peaks (FIG. 4, Parts A-B). 8214 genes were identified, where the DNA-binding of SMAD1 had decreased in the regulatory regions (FIG. 4, Part A). The search to determine if there was any overlap between these genes and the 827 genes suppressed by SB216763 in the osteoblasts was next determined (FIG. 1, Part H), and a new group of 649 genes was identified with a decrease in both gene expression and SMAD1 DNA-binding (FIG. 4, Part A). GO analysis termed these genes as involved in the signaling pathways of osteoblastic differentiation and bone formation (FIG. 4, Part A).

ChIP-seq also showed 1543 genes with increased β-catenin DNA-binding in the regulatory regions. Extended searches were conducted for potential overlaps between these genes and the cohort of 519 genes induced by SB216763 in osteoblasts (FIG. 4, Part B). A new group of 84 genes was an identified with an increase in expression and β-catenin DNA-binding (FIG. 4, Part B). GO analysis showed that these genes play roles in signaling pathways of endothelial differentiation and vessel development (FIG. 4, Part B). Collectively, the results revealed that SB216763 decreased SMAD1 and its transcriptional activity, which resulted in the loss of osteoblastic fate, and increased β-catenin and its transcriptional activity leading to endothelial differentiation.

### SB216763 Prevents Osteogenesis to Reduce Vascular Calcification

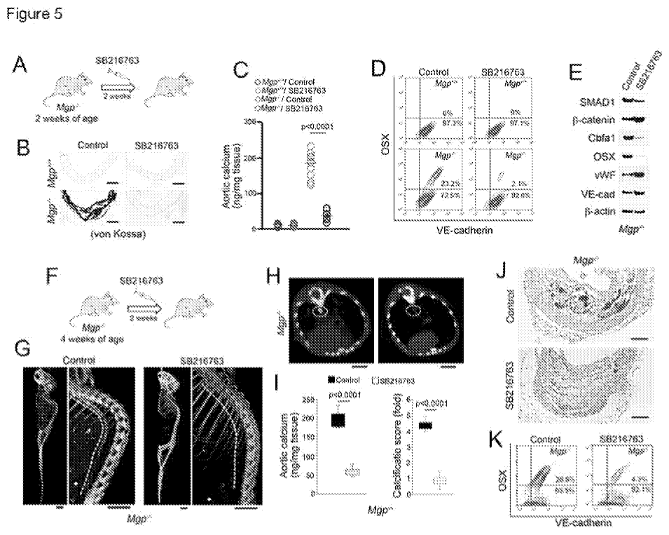

To examine whether SB216763 could be used for treatment of vascular calcification, the Mgp^{-/-} mouse was chosen, a well-known model of calcification. Mgp^{-/-} mice develop arterial calcification as early as postnatal day 14. At 4 weeks of age, the entire arterial vasculature is severely calcified. Two independent experiments were performed to test if SB216763 decreased calcification in Mgp^{-/-} mice. First, young Mgp^{-/-} mice at 2 weeks of age with SB216763 were treated (5 ug/g daily) for 2 weeks to determine if SB216763 prevented osteogenesis in the Mgp^{-/-} aortas (FIG. 5, Part A). After treatment, von Kossa staining showed a dramatic decrease in aortic mineralization (FIG. 5, Part B). The decrease in calcium deposition was confirmed by the determination of total aortic calcium in SB216763-treated Mgp^{-/-} mice (FIG. 5, Part C). Aortic cell populations expressing CD31 were isolated from mice and examined by fluorescence-activated cell sorting (FACS), which showed a decreased number of cells that co-expressed VE-cadherin and osterix but an increased number of cells that only expressed VE-cadherin in SB216763-treated Mgp^{-/-} mice (FIG. 5, Part D). Immunoblotting of aortic tissues also showed decreased Cbfa1 and osterix, increased von Willebrand factor (vWF) and VE-cadherin and altered SMAD1 and β-catenin in aortas of SB216763-treated Mgp^{-/-} mice (FIG. 5, Part E). Thus, the results demonstrated that SB216763 ameliorated vascular calcification by limiting EC-derived osteogenesis.

### SB216763 Reverses Osteogenesis Toward Endothelial Differentiation to Reduce Vascular Calcification

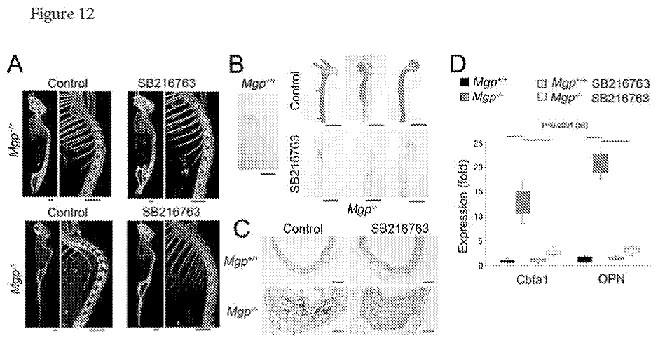

It was next examined if SB216763 reversed osteogenesis in vascular calcification by treating Mgp^{-/-} mice at 4 weeks of age for 2 weeks (FIG. 5, Part F). MicroCT imaging showed a reduction of aortic calcification with a decrease of total aortic calcium and mineral deposition in Mgp^{-/-} mice after SB216763 treatment (FIG. 5, Part G-I, FIG. 12, Part A). Alizarin red staining of whole aortic tissues also showed a reduction of mineralization in SB216763-treated Mgp^{-/-} mice (FIG. 12, Part B). Histology results demonstrated a change in morphology in calcified areas of the aortas (FIG. 5, Part J, FIG. 12, Part C). FACS showed a decrease of CD31-positive cells that co-expressed VE-cadherin and osterix, but an increase of CD31-positive cells that expressed VE-cadherin only after SB216763 treatment (FIG. 5, Part K), suggesting the osteoblastic-endothelial transdifferentiation. Again, real-time PCR showed a decrease of osteogenic markers in aortic tissue of the SB16763-treated Mgp^{-/-} mice compared to the non-treated mice (FIG. 12, Part D).

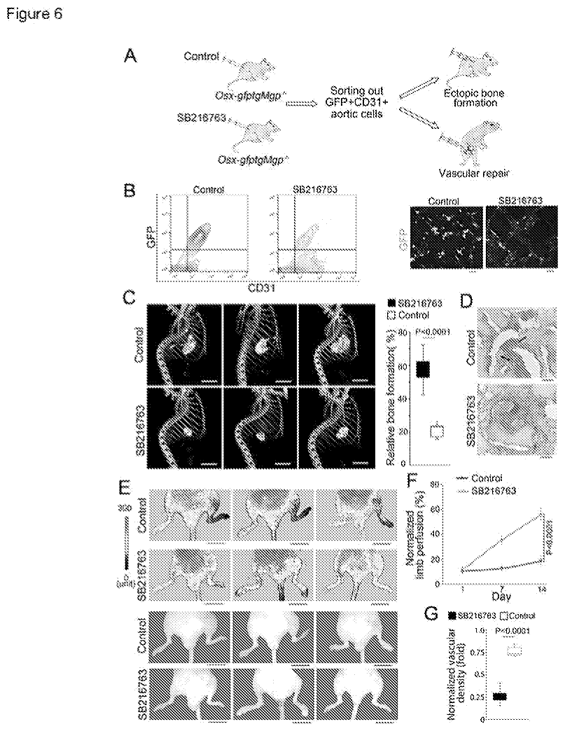

Endothelial-lineage tracing has shown the expression of osterix in osteoblast-like cells derived from labeled founder ECs in calcified aortic tissue. These osteoblast-like cells expressed both osterix and endothelial marker CD31. To further determine the shift of osteoblast-like cells toward endothelial differentiation by SB216763, osteoblastic-lineage tracing was next performed by using osterix-Gfp transgenic (Osx-Gfptg) mouse (FIG. 6, Part A), which has been broadly used to label the founder osteoblasts. Osx-GfptgMgp^{-/-} mice were bred and showed GFP and CD31 co-expression in aortic cells to confirm EC-derived osteoblast-like cell population in calcified aortic tissue (FIG. 6, Part B). Then, mice were treated with or without SB216763 for 2 weeks. FACS results showed a decreased number of cells that co-expressed GFP and CD31 but an increased number of cells that only expressed CD31 in aortic tissue of SB216763-treated group (FIG. 6, Part B), suggesting a shift of osteoblast-like cells to endothelial differentiation. Then, the alteration of GFP and CD31 double positive cells (GFP+CD31+) were characterized after SB216763 treatment. GFP+CD31+ aortic cells was isolated from Osx-GfptgMgp^{-/-} mice treated with or without SB216763 by using FACS sorting and confirmed the GFP expression (FIG. 6, Part B). Same numbers of cells of each group were used to perform a transplantation experiment to examine the capacity of bone formation by using bone formation assay in vivo. Micro-CT showed smaller size of ectopic bone, less trabecular formation and less bone volume in the implants with SB216763-treated group than controls (FIG. 6, Part C). Histology confirmed a lack of osteocytes in the implants with SB216763-treated group (FIG. 6, Part D), suggesting that osteoblast-like cells lost osteogenic capacity in aortic tissues after SB216763 treatment.

The capacity of GFP+CD31+ aortic cells for vascular repair was examined by using hindlimb ischemia model. Laser Doppler perfusion imaging showed higher limb blood flow in the mice transplanted with the cells of SB216763-treated group (FIG. 6, Part E-F). Histology analysis showed an increase in vascular density in the same group (FIG. 6, Part G), suggesting that osteoblast-like cells gain endothelial function after SB216763 treatment.

Together, the characterization of the shift of aortic osteoblast-like cells toward endothelial differentiation concluded that SB216763 induced osteoblastic-endothelial transdifferentiation to decrease vascular calcification.

### Inhibition of GSK3β Recapitulates Osteoblastic-Endothelial Transdifferentiation and Reduces Vascular Calcification

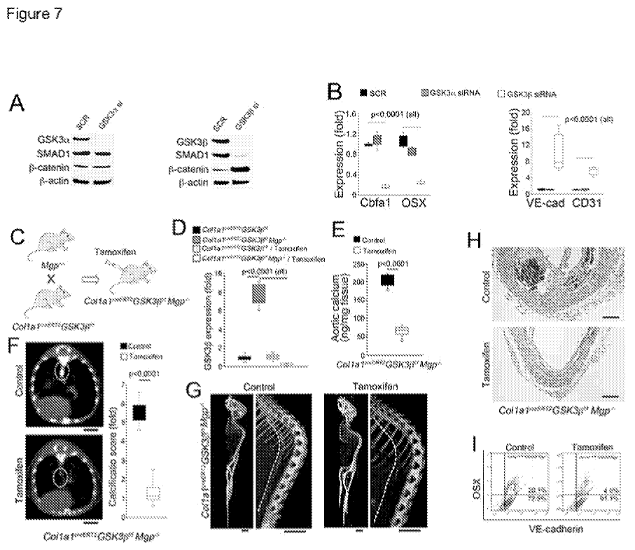

GSK3 has two isoforms GSK3α and GSK33. SB216763 specifically inhibits the activity of these GSK3 isoforms in an ATP competitive manner. To determine which isoform of GSK3 was responsible for the osteoblastic-endothelial transdifferentiation, depleted GSK3α and GSK3β was individually depleted in mouse osteoblasts using specific siRNAs (FIG. 7, Part A). The results showed a decrease of SMAD1 and osteogenic markers with an increase of β-catenin and endothelial markers only in GSK33-depleted osteoblasts (FIG. 7, Part A-B), suggesting that inhibition of GSK3β caused the osteoblastic-endothelial transdifferentiation.

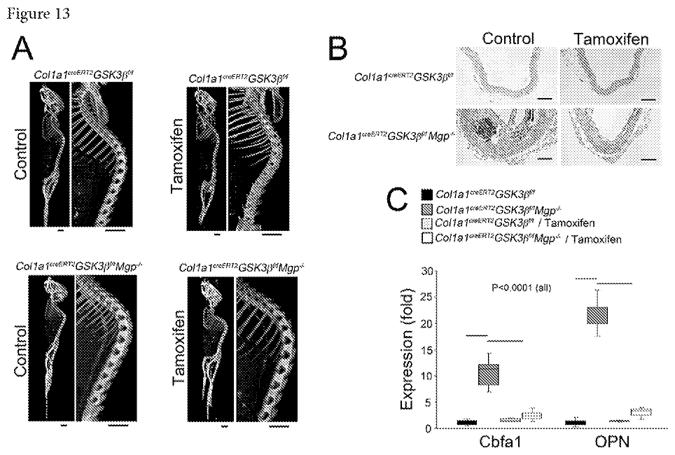

To deplete GSK3β in calcified vessels, Col1α1^{creERT2}GSK3β^{flox/flox}Mgp^{-/-} mice were generated, where tamoxifen could successfully induce Cre expression driven by the Col1α1 promoter to specifically delete GSK3β in osteoblasts. At 4 weeks of age, mice were injected with tamoxifen confirmed the reduction of GSK3β in aortic tissues (FIG. 7, Part C-D). MicroCT imaging showed a decrease of aortic calcification and total aortic calcium deposition in Col1α1^{creERT2}GSK3β^{flox/flox}Mgp^{-/-} mice after deletion of GSK3β (FIG. 7, Part E-G, FIG. 13, Part A). Histology also showed that a morphology transformation occurred in osteoblast-like cells and FACS showed an increase of endothelial cells without expression of osteogenic markers after deletion of GSK3β (FIG. 7, Part H-I, FIG. 13, Part B). Real-time PCR further revealed that deletion of GSK3β decreased osteogenic markers in aortic tissues (FIG. 13, Part C).

### Treatment of SB216763 has No Effects on Bone Formation

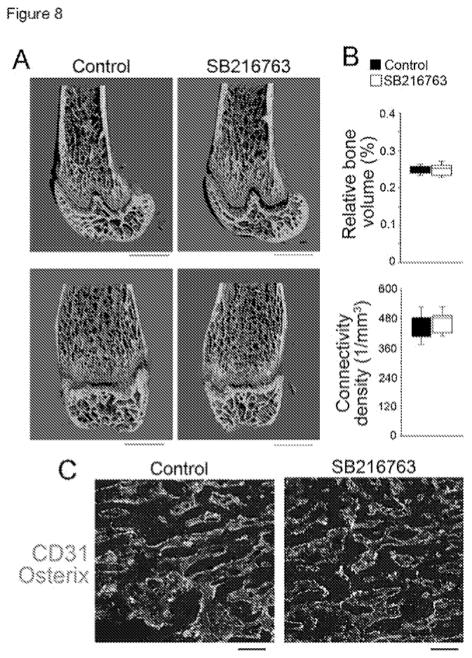

To determine if long-treatment of SB216763 has any effects on bone formation, wild type mice were treated at 8 weeks of age with SB216763 (5 ug/g daily) for 8 weeks. After treatment, the bone tissue was examined by micro-CT imaging and immunostaining. Micro-CT imaging showed no differences in relative bone volumes and connectivity densities between SB216763-treated and non-treated mice (FIG. 8, Part A-B). Immunostaining of endothelial marker CD31 and osteogenic marker osterix showed no changes in the microstructure of bone tissues and vasculature (FIG. 7, Part C). The results also had not detected any hemorrhage in the bone tissue (FIG. 8, Part C), suggesting that the treatment of SB216763 had no effects on bone formation.

In conclusion, the GSK3β inhibitor SB216763 directly switches osteoblastic fate to endothelial differentiation and reverses ectopic bone formation to ameliorates vascular calcification.

### Discussion

GSK3 inhibition switches the osteoblastic fate for endothelial differentiation by modulating SMAD1 and β-catenin, and this switch of cell fates improves vascular calcification. The results provide a new concept of osteoblastic-endothelial transdifferentiation and new information regarding the roles of GSK3 in balancing osteogenic and endothelial differentiation. The identified compound 216763 is also a new approach for the treatment of vascular calcification.

### Switch of Osteogenesis

Vascular calcification is a severe complication that increases all-cause mortality of cardiovascular disease but lacks primary medical therapy. Previously considered to be a passive process of mineral precipitation, vascular calcification is now known as an active process that involves ectopic bone formation. In this process, dysregulated systemic and local factors force vascular cells to switch cell fates for osteogenic differentiation. In diabetes mellitus, elevated by hyperglycemia, bone morphogenetic protein (BMP) signals drive vascular cells to transdifferentiate into osteoblastic-like cells causing arterial calcification. The role of endothelium in vascular calcification is not limited to be a source of osteoinductive factors responding to hyperglycemia, oscillatory shear stress or hyperlipidemia. It also directly contributes osteoprogenitor cells to calcifying process. Osteoblast-like cells with EC-origin are detected in calcified lesions of diabetic aortic tissues and atherosclerotic plaques. The studies show that, driven by endothelial-mesenchymal transition, endothelium gains plasticity for osteogenesis in vascular calcification. However, switching osteogenesis in vascular calcification has never been addressed, and is a new field of investigation. It is shown herein that it is possible to operate the switch of osteoblastic fate for endothelial differentiation and open a new direction for generating treatment strategies of calcification. The studies will benefit the patients with different types of vascular calcification or a rare disease called fibrodysplasia ossificans progressiva, in which endothelium contributes cells for osteogenesis in fibrous tissues.

With a number of advantages, small molecules have been used as a valuable tool for modulating or directing cell differentiation. Small molecules can directly modify protein or DNA to change cell differentiation and outcome phenotypes. After rationally designed screening, specific small molecules have been found to manipulate stem cell to differentiate into multiple lineages such as cardiomyocytes, neuron and hematopoietic stem cells. Treatment of small molecules also can induce pluripotency in mature cells or transdifferentiation between mature cells. Small molecules are commonly used as the approaches for mechanism studies, and expected for clinical translations. High throughput screening with a lineage reporter created a novel approach that identifies the small molecule to induce lineage transdifferentiation. This approach provides a new way to screen the candidates for correcting cell differentiation in diseases, and accelerate the identification of small molecules for translational research.

### The Role of GSK3β in Osteogenic and Endothelial Differentiation

GSK3 is a serine/threonine kinase and constitutively activated in unstimulated cells. Activity of GSK3 is regulated by serine phosphorylation in response to extracellular signals. GSK3 plays different roles in osteogenic and endothelial differentiation. GSK3 promotes the osteogenic differentiation, and GSK3 deficiency disrupts the maturation of osteoblasts resulting in the reduction of bone formation. In contrast, GSK3 prevents endothelial differentiation, and inhibition of GSK3 promotes the differentiation, proliferation and migration of ECs. GSK3 has two isoforms GSK3α and GSK3β. SB216763 is a small molecule compound that specifically inhibits the activity of GSK3 isoforms in an ATP competitive manner. SB216763 has been commonly used to probe the functions of GSK3 inhibition.

SMADs are the transcriptional factors, and have eight family members SMAD1-8. After activated by TGFβ/BMP signals, phosphorylated SMADs are translocated into nuclei to regulate the transcription of target genes. Being a critical mediator of BMP signals, the level of SMAD1 is essential for osteoblastic differentiation. Increase of SMAD1 activity promotes osteoblastic differentiation, while decrease of SMAD1 reduces osteoblastic differentiation of osteoprogenitor cells. SMAD1 protein levels are found to be regulated by GSK3 activity in sensory axon regeneration. β-catenin is a member of catenin protein family and expressed in many tissues. β-catenin is a mediator of canonical Wnt signal pathway, which is essential for EC differentiation. β-catenin also directly interacts with Notch to regulate EC specification. Because GSK3-mediated phosphorylation of β-catenin directly causes the destabilization and degradation, the activity of GSK3 is critical for modulating β-catenin level. GSK3 inhibition modulates SMAD1 and β-catenin so as to change their transcriptional activity to cause osteoblastic-endothelial transdifferentiation and reveal how GSK3 balances the transcriptional landscapes for osteogenic and endothelial differentiation.

### Methods

### Animals

Mgp^{+/-} (B6.129S7-Mgptm1Kry/KbosJ), Col1α1^{CreERT2} (B6.Cg-Tg(Col1α1-cre/ERT2)1Crm/J), GSK3β^{flox/flox} (B6.129(Cg)-Gsk3btm2Jrw/J) and Osx-Gfptg (B6.Cg-Tg(Sp7-tTA,tetO-EGFP/cre)1Amc/J) mice on C57BL/6J background were purchased from the Jackson Laboratory. Genotypes were confirmed by PCR, and experiments were performed with generation F4-F6. Littermates were used as wild type controls. All mice were fed a standard chow diet. The studies were reviewed and approved by the Institutional Review Board and conducted in accordance with the animal care guidelines set by the University of California, Los Angeles (UCLA). The investigation conformed to the National Research Council, Guide for the Care and Use of Laboratory Animals, Eighth Edition (Washington, D.C.: The National Academies Press, 2011).

### Tissue Culture

The osteoblast cell line MC3T3 was purchased from American Type Culture Collection (ATCC, CRL-2593) and cultured as per the manufacturer's protocol. SB216763 (Sigma-Aldrich, S3442) treatment was performed as described in the main text. Lentiviral vectors containing CMV-SMAD1, SMAD1 siRNA, CMV-β-catenin or β-catenin siRNA were all purchased from GeneCopeia™ and applied to the cells as per the manufacturer's protocols.

### MicroCT Imaging

MicroCT imaging was performed at the Crump Imaging Center at UCLA. All the samples were scanned on a high-resolution, volumetric microCT scanner (CT125). The image data were acquired with the following parameters: 10 μm isotropic voxel resolution; 200 ms exposure time; 2,000 views and 5 frames per view. The microCT-generated DICOM files were used to analyze the samples and to create volume renderings of the regions of interest. The raw data files were viewed using the MicroView 3-D volume viewer and analysis tool (GE Healthcare) and AltaViewer™ Software. Additionally, images of the samples were generated using SCIRun (Scientific Computing and Imaging Institute).

### Laser Doppler Perfusion Image

Laser Doppler perfusion imaging was performed using real-time microcirculation imaging system (Perimed). The imaging was conducted under normal ambient room lighting. 20×27 mm high resolution model was used with 1388×1038 pixels measurement camera and 752×580 pixels documentation camera as one image per second. The image resolution was set up as 20 μm/pixel and 21 images per frame until stopped. Windows based PIMSoft software (Perimed) was used to process the data.

### Mouse Surgery

All the surgeries were performed on a heated pad with a connection to a continuous flow of isoflurane. Ectopic bone formation was performed as previously described: 5×10⁵ cells were mixed with 40 mg hydroxyapatite/tricalcium phosphate power (SALVIN™, ORASTRUCT-0.5 CC) and incubated in a 1 ml syringe at 37° C. in 5% CO₂ overnight. After disinfection with 70% ethanol, a skin incision was made on the back of mouse. A subcutaneous pouch was formed by blunt dissection. The mixture of cells and hydroxyapatite/tricalcium phosphate was transplanted into the pouch and the incision was closed. The implants were examined by microCT imaging and histology 12 weeks after transplantation.

The murine model of hindlimb ischemia was performed as previously described. A 10 mm long incision of the skin was made towards the medial thigh. The femoral artery was exposed and separated from femoral vein and nerve. Silk sutures were used to tie the proximal and the distal end of femoral artery with double knots. The cells (5×10⁵) were transplanted into the surgical area and the incision was closed. Laser Doppler perfusion imaging was used to monitor the blood flow at different time points. Histology and immunostaining were used to examine the vascularization 2 weeks after transplantation.

### RNA Analysis

Real-time PCR analysis was performed as previously described. Glyceraldehyde 3-phosphate dehydrogenase (GAPDH) was used as a control gene. Primers and probes for mouse and human Cbfa1, ostrix, osteocalcin, CD34, VE-cadherin, CD31, eNOS, SMAD1, and β-catenin, were obtained from Applied Biosystems as part of Tagman® Gene Expression Assays.

### Fluorescence-Activated Cell Sorting (FACS)

FACS analysis was performed as previously described. The cells were stained with fluorescein isothiocyanate (FITC)-, phycoerythrin (PE)-, or Alexa Fluor 488 (AF-488)-conjugated antibodies against CD31 and VE-cadherin (all from BD biosciences, 550274 and 562243), osterix (Santa Cruz Biotechnology, sc-22536). Nonspecific fluorochrome- and isotype-matched IgGs (BD Pharmingen) served as controls.

### Immunoblotting and Immunofluorescence

Immunoblotting was performed as previously described. Equal amounts of tissue lysates were used for immunoblotting. Blots were incubated with specific antibodies to SMAD1, GSK3α and GSK3β (all from Cell Signaling Technology, 9743, 433T and 93115), β-catenin and cbfa1 (all from R&D system, AF1329 and MAB2006), osterix (Santa Cruz Biotechnology, sc-22536), Flk1 and VE-cadherin (all from BD Bioscience, 55307 and 562242), vWF (Dako, A0082). 3-Actin (Sigma-Aldrich, A2228) was used as a loading control. Immunofluorescence was performed as previously described in detail. Specific antibodies to CD31 (BD Bioscience, 553370), osterix (Santa Cruz Biotechnology, sc-22536) and vWF (Dako, A0082) were used. The nuclei were stained with 4′,6-diamidino-2-phenylindole (DAPI, Sigma-Aldrich, D9564).

### RNA-seq, ChIP-seq and ChIP-Assay

For RNA sequencing, osteoblasts were treated with 10 μM SB216763 for 14 days, and RNA was isolated for library preparation. The sequencing was conducted by the Pathology Research Services at UCLA. Spliced Transcripts Alignment to Reference (STAR) was used for the read alignment. Cufflinks was used to assemble transcripts, estimate their abundances, and assess the differential expression. GO analysis and pathway enrichment of the identified genes were performed.

For ChIP-seq, specific antibodies were used to enrich the genomic DNA as described before. ChIP DNA were sequenced by the Pathology Research Services at UCLA. Reads from each sample were mapped to the mouse genome using Bowtie2. Homer tool was used to detect significant enrichment of peaks with 5% false discovery rate and >4-fold over input. Motif occurrences in peaks were identified by the Homer motif discovery function. Peak annotation was performed to associate peaks with nearby genes and calculate tag densities. GO analysis and pathway enrichment of the identified genes were also performed. Specific antibodies for SMAD1 (Cell Signaling Technology, 9743) and β-catenin (R&D System, MAB2006) were used. The data were deposited in Gene Expression Omnibus (GEO) database with access number (GSE147374).

βChIP assays were performed as previously described. Specific antibodies for β-catenin were used (R&D System, MAB2006), H3k4me3 (Abcam, ab8580) and H3k27me3 (Abcam, ab6002). The primers for the real-time PCR: 5′GAAAATAACACAGGCTTTG3′ (SEQ ID NO: 13) and 5′ GCTCCCCGAGCCTGGATT 3′ (SEQ ID NO: 14); 5′ GGACAGAGGCTCTCATTCC 3′ (SEQ ID NO: 15) and 5′CAATTCTTGGATCTCATCTTA3′ (SEQ ID NO: 16) and 5′GGGTGACCAAGCATGCTAGC3′ (SEQ ID NO: 17) 5′CCTGGCCACCTCCATCTTGC3′ (SEQ ID NO: 18); 5′GGAGAGGCCATGTTGAGGAC3′ (SEQ ID NO: 19) and 5′CCTAGCGTCTACACTGGGTAG3′ (SEQ ID NO: 20).

### High Throughput System for the Compound Screen

MC3T3 cells were stably infected with Flk1 promoter-driven eGFP by using Flk1-eGFP lentivirus (GeneCopoeia™). The plates were coated with laminin (20 ug/ml) and washed with PBS twice using an ELx 405 plate washer (Bio-Tek Instruments). Cells in 25 μl medium per well were loaded by Multidrop 384 (Thermo LabSystems), and the chemical compounds were pinned to the plates with media. GFP positive cells (positive controls) and wild type cells (negative controls) were also seeded. The plates were transferred to a STX 220 CO₂ plate incubator (Liconic Instruments) and incubated. The plates were transferred and delivered by a Thermo Scientific™ Spinnaker™ Robot (ThermoFisher Scientific). eGFP expression was determined and imaged using a FlexStation II and Victor 3V (Perkin Elmer) every day for two weeks.

### Statistical Analysis

The analyses were performed using GraphPad Instat®, version 3.0 (GraphPad Software). Data were analyzed by either unpaired 2-tailed Student's t test or one-way ANOVA with Tukey's multiple-comparisons test for statistical significance.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments are described herein.

Such equivalents are intended to be encompassed by the following claims.

1. A method of treating or preventing vascular calcification inducing or increasing osteoblastic-endothelial transdifferentiation, or inhibiting or decreasing osteogenesis in a subject in need thereof, comprising administering to the subject an agent that inhibits the activity of or decreases the levels of glycogen synthase kinase 3 (GSK3).

2-3. (canceled)

4. The method of claim 1, wherein the GSK3 is GSK3β.

5. A method of treating or preventing vascular calcification, inducing or increasing osteoblastic-endothelial transdifferentiation, or inhibiting or decreasing osteogenesis in a subject in need thereof, comprising administering to the subject an agent that inhibits the activity of or decreases the levels of mothers against decapentaplegic homolog 1 (SMAD1).

6-7. (canceled)

8. A method of treating or preventing vascular calcification, inducing or increasing osteoblastic-endothelial transdifferentiation, or inhibiting or decreasing osteogenesis in a subject in need thereof, comprising administering to the subject an agent that activates or increases the levels of β-catenin.

9-10. (canceled)

11. The method of claim 1, wherein the agent is a small molecule.

12. The method of claim 11, wherein the small molecule is SB216763.

13. The method of claim 1, wherein the agent is a polypeptide.

14. The method of claim 1, wherein the agent is an inhibitory polynucleotide specific for aft GSK3 protein.

15. The method of claim 14, wherein the GSK3 is GSK33.

16. The method of claim 14, wherein the inhibitory polynucleotide is selected from siRNA, shRNA, and an antisense RNA molecule, or a polynucleotide that encodes a molecule selected from siRNA, shRNA, and/or an antisense RNA molecule.

17. The method of claim 5, wherein the agent is an inhibitory polynucleotide specific for an SMAD1 protein.

18. The method of claim 17, wherein the inhibitory polynucleotide is selected from siRNA, shRNA, and an antisense RNA molecule, or a polynucleotide that encodes a molecule selected from siRNA, shRNA, and/or an antisense RNA molecule.

19. The method of claim 8, wherein the agent is a polynucleotide encoding a β-catenin protein.

20-52. (canceled)

53. The method of claim 1, wherein the subject is afflicted with cardiovascular disease.

54. The method of claim 1, wherein the subject is afflicted with chronic kidney disease.

55. The method of claim 1, wherein the subject is afflicted with diabetes mellitus.

56. The method of claim 1, wherein the subject is afflicted with fibrodysplasia ossificans progressiva.


SEQUENCE LISTING (20 sequences)

SEQ ID NO: 1 - DNA, 7743 bp, Homo sapiens
acacagagcagctcctgacccgggcgaatgcgggcttgtgccgccgccgccgccgccgccgcccgggccaagtgacaaaggaaggaaggaagcgaggaggagccggccccgcagccgctgacagggctctgggctggggcaaagcgcggacacttcctgagcgggcaccgagcagagccgaggggcgggagggcggccgagctgttgccgcggacgggggagggggccccgagggacggaagcggttgccgggttcccatgtccccggcgaatggggaacagtcgaggagccgctgcctggggtctgaagggagctgcctccgccaccgccatggccgctggatccagccgccgcctgcagctgctcctggcgcaatgaggagaggagccgccgccaccgccaccgcccgcctctgactgactcgcgactccgccgccctctagttcgccgggcccctgccgtcagcccgccggatcccgcggcttgccggagctgcagcgtttcccgtcgcatctccgagccaccccctccctccctctccctccctcctacccatccccctttctcttcaagcgtgagactcgtgatccttccgccgcttcccttcttcattgactcggaaaaaaaatccccgaggaaaatataatattcgaagtactcattttcaatcaagtatttgcccccgtttcacgtgatacatatttttttaggatttgccctctcttttctctcctcccaggaaagggaggggaaagaattgtattttttcccaagtcctaaatcatctatatgttaaatatccgtgccgatctgtcttgaaggagaaatatatcgcttgttttgttttttatagtatacaaaaggagtgaaaagccaagaggacgaagtctttttctttttcttctgtgggagaacttaatgctgcatttatcgttaacctaacaccccaacataaagacaaaaggaagaaaaggaggaaggaaggaaaaggtgattcgcgaagagagtgatcatgtcagggcggcccagaaccacctcctttgcggagagctgcaagccggtgcagcagccttcagcttttggcagcatgaaagttagcagagacaaggacggcagcaaggtgacaacagtggtggcaactcctgggcagggtccagacaggccacaagaagtcagctatacagacactaaagtgattggaaatggatcatttggtgtggtatatcaagccaaactttgtgattcaggagaactggtcgccatcaagaaagtattgcaggacaagagatttaagaatcgagagctccagatcatgagaaagctagatcactgtaacatagtccgattgcgttatttcttctactccagtggtgagaagaaagatgaggtctatcttaatctggtgctggactatgttccggaaacagtatacagagttgccagacactatagtcgagccaaacagacgctccctgtgatttatgtcaagttgtatatgtatcagctgttccgaagtttagcctatatccattcctttggaatctgccatcgggatattaaaccgcagaacctcttgttggatcctgatactgctgtattaaaactctgtgactttggaagtgcaaagcagctggtccgaggagaacccaatgtttcgtatatctgttctcggtactatagggcaccagagttgatctttggagccactgattatacctctagtatagatgtatggtctgctggctgtgtgttggctgagctgttactaggacaaccaatatttccaggggatagtggtgtggatcagttggtagaaataatcaaggtcctgggaactccaacaagggagcaaatcagagaaatgaacccaaactacacagaatttaaattccctcaaattaaggcacatccttggactaaggtcttccgaccccgaactccaccggaggcaattgcactgtgtagccgtctgctggagtatacaccaactgcccgactaacaccactggaagcttgtgcacattcattttttgatgaattacgggacccaaatgtcaaactaccaaatgggcgagacacacctgcactcttcaacttcaccactcaagaactgtcaagtaatccacctctggctaccatccttattcctcctcatgctcggattcaagcagctgcttcaacccccacaaatgccacagcagcgtcagatgctaatactggagaccgtggacagaccaataatgctgcttctgcatcagcttccaactccacctgaacagtcccgagcagccagctgcacaggaaaaaccaccagttacttgagtgtcactcagcaacactggtcacgtttggaaagaatattaaaaagagaaaaaaatcctgttcattttagtgttcaatttttttattattattgttgttcttatttaaccttgtaaaatatctataaatacaaaccaatttcattgtattctcactttgagggagatccagggggtgggaggggttgtggggagggggaaagcggagcactagaacatacaatctctctcccacgacaatctttttttattaaaagtctgctgttgtatactttaaaaacaggactcctgcctcatgccccttccacaaaagaagaaaacctttttctgtgctgatgggtttttttgaactttgttttcttttaaagtctagtgtgagactttggtatagtgcacagcttgaaattggttgggagcttagcaggtataactcaacggggacttaaatgtcacttgtaaaattaatccatatcttcgggtatttatagacttgcctttggcatgttggtggcaggtgtggcagacaaagaaatgtgtatcattcgtaacccagggaggtcaataaagtttggaactctacagggaagattcttagtagatttgttaaggttttgttttgctctcagttagtgctagtgatgtagaggcttgtacaggaggctgccagaggggaagcagcaagcaagactcaggcacacatgctctacaggtggctctttgtttgcctgaccaaagttctttgcaaatcttagcacagtttcaaactagtgacctgggaggagatggaaggggtgttgagcaggctgagctagctgctgaggtcaaaggctgatgagcccagaggaaggggacaggtcagggatacatctcaccactgtgaataagtttgtccagatttttttctaaagttacttcccttggaaagatacacttgagaggacattgtagttaaataatgtgaactgtaacagtcatctactggtttatttttcatattttttaattgaaaattgagcttgcagaaatagccacattctacacatagttctaattttaaatccaaatctagaatctgtatttaatttgttttttaacctcatgctttttacatttatttattgatgcatgtcagatggtagaaatattaaaaactacacatcagaatgatacagtcacttatacctgctgactttataggaaagctgatgatataaatgtgtgtatatatgttatatatacatatattcaatactgcctttttttttgtctacagtatcaaaattgactggttgaagcatgagaagaatgtttcccccacacccagttaagagtttttgtgtctgttttctttgtgtatcagtgaacgatgttaagaatcagtctctctttttgaagaaaaagcaatattccttggaaagcaaggagaattgaaggactatgtttgccgtgaggaaatagattttcatgactagtttgttttatacttttaaggttggcatctatgtgggccttatatactctaaaatgaactttagtcaccttggtgcttatgggccattacttgacctatgaatctttaaggcacaatcagttgtactttacatttaaagatcacttgagtgatggccgcctttccctcctacccgctccttccccacatgccttccaaggttagctggtaactgtagggctgcagagctgagcccatggttgtgtgtaacttgccctcaccctcctcattgccaccttaggtcactttatgggtctcgtcctccagagggttcggaagtggagtctgttggcagccctcctgcaggccctagcaccctgtcctgctccttaactgtgtgtgtgactctccaagagagttgtcctgcctgctgaagtgaaccagtacccagaaagacaactgtgagccatcttggttttcactcgctgtttagctgaggtcttgggccacaaaaggggtttcacaaacctctggatatatcagagtttatgagaaaggaaacatgctcagtcaaaccaaatcaaacaaattgaattttatgttttataaagtgcttctgaaagctaagatttgaaagaagtctgaaatcaaagtatttggcagcataactccttaaaggtagtggcgttgatagaccattttcagacagaatttataaagaatctgaaaaggcaggtctgtgatagagaaatggacctgcattcagatccaactgcccagcaagcgtttggatgcagacactgctctggacgtggtatactccccagagtccataaaaatcagtgcttattttaggaaacaggttgccccccacaactggggtaaaagaagagagaaaagtcacgcttttctctcatttcattgtgtgtgcatgtgtgcgtgtgtgtgtgtgtgtgtgtgtgctgagatgtgtgatttttctttctcaaggatcatggtgggatcacagaactcttttatacaagtgagatccaggtctctgaatatctttttgtatataataataataaaaagctcctcaccaaattcaagcttgtacattatattttctttctgtgtttttaaatttaagttttattgttttgtatgtaaatatgtggacccaggaactgttattaatgagcaaaaagttactgttcagggcagtgattctgtttaataatcagacaaaatgtagacgagctttttaaagccatatagttttaactctgtacagtaggtaccggcctgtattattgtaacaataactctagcaatgtatagtgtatctatatagtttggagtgccttcgcttccatgtgtttttttttttaatttgttcttttttaaattttaattggtttcctttatccatgtctccctgtccaccccctttccctttgaaataataactcactcataacagtatctttgccccttccacagttaagtttcagtgataccatactcaggagtgggaagaggaaatcatattcgtaatttcatttcgttgaagccctgcctttgttttggttctgaatgtctttcctcctcggtagcagtgagaccggtttcatttcatacttagtccattcagggacttagtgtagcaccagggagccctagagctggaggatatcgaatagattaaattttgctcgtctcttccacaagccctaaccatgggtcttaaaaacagcagattctgggagccttccatgctctctctctctcctcttttatctacttccctcccaaatgagagagtgacagagaattgtttttttataaatcgaagtttcttaatagtatcaggttttgatacgtcagtggtctaaaatgctatagtgcaattactagcagttactgcacggagtgccaccgtgccaatagaggactgttgttttaacaagggaactcttagcccatttcctccctcccgccatctctacccttgctcaatgaaatatcattttaatttcttttaaaaaaaatcagtttaattcttactgtgtgcccaacacgaaggccttttttgaaagaaaaatagaatgttttgcctcaaagtagtccatataaaatgtcttgaatagaagaaaaaactaccaaaccaaaggttactatttttgaaacatcgtgtgttcattccagcaaggcagaagactgcaccttctttccagtgacatgctgtgtcattttttttaagtcctcttaatttttagacacatttttggtttatgttttaacaatgtatgcctaaccagtcatcttgtctgcaccaatgcaaaggtttctgagaggagtattctctatccctgtggatatgaagacactggcatttcatctatttttccctttcctttttaaaggatttaactttggaatcttccaaaggaagtttggccaatgccagatccccaggaatttggggggttttctttcttttcaactgaaattgtatctgattcctactgttcatgttagtgatcatctaatcacagagccaaacacttttctcccctgtgtggaaaagtaggtatgctttacaataaaatctgtcttttctggtagaaacctgagccactgaaaataaaagagacaactagaagcacagtagagtcccagactgagatctacctttgagaggctttgaaagtaatccctggggtttggattattttcacaagggttatgccgttttattcaagtttgttgctccgttttgcacctctgcaataaaagcaaaatgacaaccagtacataaggggttagcttgacaaagtagacttccttgtgttaatttttaagtttttttttccttaactatatctgtctacaggcagatacagatagttgtatgaaaatctgcttgcctgtaaaatttgcatttataaatgtgttgccgatggatcacttgggcctgtacacataccaattagcgtgaccacttccatcttaaaaacaaacctaaaaaacaaaatttattatatatatatatatatatatataaaggactgtgggttgtatacaaactattgcaaacacttgtgcaaatctgtcttgatataaaggaaaagcaaaatctgtataacattattactacttgaatgcctctgtgactgatttttttttcattttaaatataaacttttttgtgaaaagtatgctcaatgttttttttccctttccccattcccttgtaaatacattttgttctatgtgacttggtttggaaatagttaactggtactgtaatttgcattaaataaaaagtaggttagcctggaaatgaaattaaaattcacaagtgtgtggtctttatttcagtacccaaccctcttccttcaccctactattttgccgctgcaatatgtagtcacatcaccatttccattcctctaattagggaaacattaatctttgttatacagaacaagatatcaataccacttcttgttctttccaatgattttattccattgtgtagccccaagaggtgcagcttccaccttggaaacctttggatttgatgtagaggaagctttgcagacactgcttagaaaagaaagaaaacaactctgaaagggacagtttttaaatgtgtataagctgctgtctttgattactgtgttcatgatttggtgtggctgtattttcttttaactttcatcctattagtaatggtctttgggggtctctgtaaaatatatggacaccacgaacagtggggctgtacctcccaggtaaccaacacatgttgtgtttgagtctgctcatttccaatactggatgatgtatgtaaacatgttatgtctcttagtgcaaaaagaaacatcatttttttagggctggctcactctgtcaggcctaatctaaaggctagatataaggtcatgtgactgctgcttcaataaaaacaaatttatattcga

SEQ ID NO: 2 - DNA, 7782 bp, Homo sapiens
acacagagcagctcctgacccgggcgaatgcgggcttgtgccgccgccgccgccgccgccgcccgggccaagtgacaaaggaaggaaggaagcgaggaggagccggccccgcagccgctgacagggctctgggctggggcaaagcgcggacacttcctgagcgggcaccgagcagagccgaggggcgggagggcggccgagctgttgccgcggacgggggagggggccccgagggacggaagcggttgccgggttcccatgtccccggcgaatggggaacagtcgaggagccgctgcctggggtctgaagggagctgcctccgccaccgccatggccgctggatccagccgccgcctgcagctgctcctggcgcaatgaggagaggagccgccgccaccgccaccgcccgcctctgactgactcgcgactccgccgccctctagttcgccgggcccctgccgtcagcccgccggatcccgcggcttgccggagctgcagcgtttcccgtcgcatctccgagccaccccctccctccctctccctccctcctacccatccccctttctcttcaagcgtgagactcgtgatccttccgccgcttcccttcttcattgactcggaaaaaaaatccccgaggaaaatataatattcgaagtactcattttcaatcaagtatttgcccccgtttcacgtgatacatatttttttaggatttgccctctcttttctctcctcccaggaaagggaggggaaagaattgtattttttcccaagtcctaaatcatctatatgttaaatatccgtgccgatctgtcttgaaggagaaatatatcgcttgttttgttttttatagtatacaaaaggagtgaaaagccaagaggacgaagtctttttctttttcttctgtgggagaacttaatgctgcatttatcgttaacctaacaccccaacataaagacaaaaggaagaaaaggaggaaggaaggaaaaggtgattcgcgaagagagtgatcatgtcagggcggcccagaaccacctcctttgcggagagctgcaagccggtgcagcagccttcagcttttggcagcatgaaagttagcagagacaaggacggcagcaaggtgacaacagtggtggcaactcctgggcagggtccagacaggccacaagaagtcagctatacagacactaaagtgattggaaatggatcatttggtgtggtatatcaagccaaactttgtgattcaggagaactggtcgccatcaagaaagtattgcaggacaagagatttaagaatcgagagctccagatcatgagaaagctagatcactgtaacatagtccgattgcgttatttcttctactccagtggtgagaagaaagatgaggtctatcttaatctggtgctggactatgttccggaaacagtatacagagttgccagacactatagtcgagccaaacagacgctccctgtgatttatgtcaagttgtatatgtatcagctgttccgaagtttagcctatatccattcctttggaatctgccatcgggatattaaaccgcagaacctcttgttggatcctgatactgctgtattaaaactctgtgactttggaagtgcaaagcagctggtccgaggagaacccaatgtttcgtatatctgttctcggtactatagggcaccagagttgatctttggagccactgattatacctctagtatagatgtatggtctgctggctgtgtgttggctgagctgttactaggacaaccaatatttccaggggatagtggtgtggatcagttggtagaaataatcaaggtcctgggaactccaacaagggagcaaatcagagaaatgaacccaaactacacagaatttaaattccctcaaattaaggcacatccttggactaaggattcgtcaggaacaggacatttcacctcaggagtgcgggtcttccgaccccgaactccaccggaggcaattgcactgtgtagccgtctgctggagtatacaccaactgcccgactaacaccactggaagcttgtgcacattcattttttgatgaattacgggacccaaatgtcaaactaccaaatgggcgagacacacctgcactcttcaacttcaccactcaagaactgtcaagtaatccacctctggctaccatccttattcctcctcatgctcggattcaagcagctgcttcaacccccacaaatgccacagcagcgtcagatgctaatactggagaccgtggacagaccaataatgctgcttctgcatcagcttccaactccacctgaacagtcccgagcagccagctgcacaggaaaaaccaccagttacttgagtgtcactcagcaacactggtcacgtttggaaagaatattaaaaagagaaaaaaatcctgttcattttagtgttcaatttttttattattattgttgttcttatttaaccttgtaaaatatctataaatacaaaccaatttcattgtattctcactttgagggagatccagggggtgggaggggttgtggggagggggaaagcggagcactagaacatacaatctctctcccacgacaatctttttttattaaaagtctgctgttgtatactttaaaaacaggactcctgcctcatgccccttccacaaaagaagaaaacctttttctgtgctgatgggtttttttgaactttgttttcttttaaagtctagtgtgagactttggtatagtgcacagcttgaaattggttgggagcttagcaggtataactcaacggggacttaaatgtcacttgtaaaattaatccatatcttcgggtatttatagacttgcctttggcatgttggtggcaggtgtggcagacaaagaaatgtgtatcattcgtaacccagggaggtcaataaagtttggaactctacagggaagattcttagtagatttgttaaggttttgttttgctctcagttagtgctagtgatgtagaggcttgtacaggaggctgccagaggggaagcagcaagcaagactcaggcacacatgctctacaggtggctctttgtttgcctgaccaaagttctttgcaaatcttagcacagtttcaaactagtgacctgggaggagatggaaggggtgttgagcaggctgagctagctgctgaggtcaaaggctgatgagcccagaggaaggggacaggtcagggatacatctcaccactgtgaataagtttgtccagatttttttctaaagttacttcccttggaaagatacacttgagaggacattgtagttaaataatgtgaactgtaacagtcatctactggtttatttttcatattttttaattgaaaattgagcttgcagaaatagccacattctacacatagttctaattttaaatccaaatctagaatctgtatttaatttgttttttaacctcatgctttttacatttatttattgatgcatgtcagatggtagaaatattaaaaactacacatcagaatgatacagtcacttatacctgctgactttataggaaagctgatgatataaatgtgtgtatatatgttatatatacatatattcaatactgcctttttttttgtctacagtatcaaaattgactggttgaagcatgagaagaatgtttcccccacacccagttaagagtttttgtgtctgttttctttgtgtatcagtgaacgatgttaagaatcagtctctctttttgaagaaaaagcaatattccttggaaagcaaggagaattgaaggactatgtttgccgtgaggaaatagattttcatgactagtttgttttatacttttaaggttggcatctatgtgggccttatatactctaaaatgaactttagtcaccttggtgcttatgggccattacttgacctatgaatctttaaggcacaatcagttgtactttacatttaaagatcacttgagtgatggccgcctttccctcctacccgctccttccccacatgccttccaaggttagctggtaactgtagggctgcagagctgagcccatggttgtgtgtaacttgccctcaccctcctcattgccaccttaggtcactttatgggtctcgtcctccagagggttcggaagtggagtctgttggcagccctcctgcaggccctagcaccctgtcctgctccttaactgtgtgtgtgactctccaagagagttgtcctgcctgctgaagtgaaccagtacccagaaagacaactgtgagccatcttggttttcactcgctgtttagctgaggtcttgggccacaaaaggggtttcacaaacctctggatatatcagagtttatgagaaaggaaacatgctcagtcaaaccaaatcaaacaaattgaattttatgttttataaagtgcttctgaaagctaagatttgaaagaagtctgaaatcaaagtatttggcagcataactccttaaaggtagtggcgttgatagaccattttcagacagaatttataaagaatctgaaaaggcaggtctgtgatagagaaatggacctgcattcagatccaactgcccagcaagcgtttggatgcagacactgctctggacgtggtatactccccagagtccataaaaatcagtgcttattttaggaaacaggttgccccccacaactggggtaaaagaagagagaaaagtcacgcttttctctcatttcattgtgtgtgcatgtgtgcgtgtgtgtgtgtgtgtgtgtgtgctgagatgtgtgatttttctttctcaaggatcatggtgggatcacagaactcttttatacaagtgagatccaggtctctgaatatctttttgtatataataataataaaaagctcctcaccaaattcaagcttgtacattatattttctttctgtgtttttaaatttaagttttattgttttgtatgtaaatatgtggacccaggaactgttattaatgagcaaaaagttactgttcagggcagtgattctgtttaataatcagacaaaatgtagacgagctttttaaagccatatagttttaactctgtacagtaggtaccggcctgtattattgtaacaataactctagcaatgtatagtgtatctatatagtttggagtgccttcgcttccatgtgtttttttttttaatttgttcttttttaaattttaattggtttcctttatccatgtctccctgtccaccccctttccctttgaaataataactcactcataacagtatctttgccccttccacagttaagtttcagtgataccatactcaggagtgggaagaggaaatcatattcgtaatttcatttcgttgaagccctgcctttgttttggttctgaatgtctttcctcctcggtagcagtgagaccggtttcatttcatacttagtccattcagggacttagtgtagcaccagggagccctagagctggaggatatcgaatagattaaattttgctcgtctcttccacaagccctaaccatgggtcttaaaaacagcagattctgggagccttccatgctctctctctctcctcttttatctacttccctcccaaatgagagagtgacagagaattgtttttttataaatcgaagtttcttaatagtatcaggttttgatacgtcagtggtctaaaatgctatagtgcaattactagcagttactgcacggagtgccaccgtgccaatagaggactgttgttttaacaagggaactcttagcccatttcctccctcccgccatctctacccttgctcaatgaaatatcattttaatttcttttaaaaaaaatcagtttaattcttactgtgtgcccaacacgaaggccttttttgaaagaaaaatagaatgttttgcctcaaagtagtccatataaaatgtcttgaatagaagaaaaaactaccaaaccaaaggttactatttttgaaacatcgtgtgttcattccagcaaggcagaagactgcaccttctttccagtgacatgctgtgtcattttttttaagtcctcttaatttttagacacatttttggtttatgttttaacaatgtatgcctaaccagtcatcttgtctgcaccaatgcaaaggtttctgagaggagtattctctatccctgtggatatgaagacactggcatttcatctatttttccctttcctttttaaaggatttaactttggaatcttccaaaggaagtttggccaatgccagatccccaggaatttggggggttttctttcttttcaactgaaattgtatctgattcctactgttcatgttagtgatcatctaatcacagagccaaacacttttctcccctgtgtggaaaagtaggtatgctttacaataaaatctgtcttttctggtagaaacctgagccactgaaaataaaagagacaactagaagcacagtagagtcccagactgagatctacctttgagaggctttgaaagtaatccctggggtttggattattttcacaagggttatgccgttttattcaagtttgttgctccgttttgcacctctgcaataaaagcaaaatgacaaccagtacataaggggttagcttgacaaagtagacttccttgtgttaatttttaagtttttttttccttaactatatctgtctacaggcagatacagatagttgtatgaaaatctgcttgcctgtaaaatttgcatttataaatgtgttgccgatggatcacttgggcctgtacacataccaattagcgtgaccacttccatcttaaaaacaaacctaaaaaacaaaatttattatatatatatatatatatatataaaggactgtgggttgtatacaaactattgcaaacacttgtgcaaatctgtcttgatataaaggaaaagcaaaatctgtataacattattactacttgaatgcctctgtgactgatttttttttcattttaaatataaacttttttgtgaaaagtatgctcaatgttttttttccctttccccattcccttgtaaatacattttgttctatgtgacttggtttggaaatagttaactggtactgtaatttgcattaaataaaaagtaggttagcctggaaatgaaattaaaattcacaagtgtgtggtctttatttcagtacccaaccctcttccttcaccctactattttgccgctgcaatatgtagtcacatcaccatttccattcctctaattagggaaacattaatctttgttatacagaacaagatatcaataccacttcttgttctttccaatgattttattccattgtgtagccccaagaggtgcagcttccaccttggaaacctttggatttgatgtagaggaagctttgcagacactgcttagaaaagaaagaaaacaactctgaaagggacagtttttaaatgtgtataagctgctgtctttgattactgtgttcatgatttggtgtggctgtattttcttttaactttcatcctattagtaatggtctttgggggtctctgtaaaatatatggacaccacgaacagtggggctgtacctcccaggtaaccaacacatgttgtgtttgagtctgctcatttccaatactggatgatgtatgtaaacatgttatgtctcttagtgcaaaaagaaacatcatttttttagggctggctcactctgtcaggcctaatctaaaggctagatataaggtcatgtgactgctgcttcaataaaaacaaatttatattcga

SEQ ID NO: 3 - DNA, 7644 bp, Homo sapiens
acacagagcagctcctgacccgggcgaatgcgggcttgtgccgccgccgccgccgccgccgcccgggccaagtgacaaaggaaggaaggaagcgaggaggagccggccccgcagccgctgacagggctctgggctggggcaaagcgcggacacttcctgagcgggcaccgagcagagccgaggggcgggagggcggccgagctgttgccgcggacgggggagggggccccgagggacggaagcggttgccgggttcccatgtccccggcgaatggggaacagtcgaggagccgctgcctggggtctgaagggagctgcctccgccaccgccatggccgctggatccagccgccgcctgcagctgctcctggcgcaatgaggagaggagccgccgccaccgccaccgcccgcctctgactgactcgcgactccgccgccctctagttcgccgggcccctgccgtcagcccgccggatcccgcggcttgccggagctgcagcgtttcccgtcgcatctccgagccaccccctccctccctctccctccctcctacccatccccctttctcttcaagcgtgagactcgtgatccttccgccgcttcccttcttcattgactcggaaaaaaaatccccgaggaaaatataatattcgaagtactcattttcaatcaagtatttgcccccgtttcacgtgatacatatttttttaggatttgccctctcttttctctcctcccaggaaagggaggggaaagaattgtattttttcccaagtcctaaatcatctatatgttaaatatccgtgccgatctgtcttgaaggagaaatatatcgcttgttttgttttttatagtatacaaaaggagtgaaaagccaagaggacgaagtctttttctttttcttctgtgggagaacttaatgctgcatttatcgttaacctaacaccccaacataaagacaaaaggaagaaaaggaggaaggaaggaaaaggtgattcgcgaagagagtgatcatgtcagggcggcccagaaccacctcctttgcggagagctgcaagccggtgcagcagccttcagcttttggcagcatgaaagttagcagagacaaggacggcagcaaggtgacaacagtggtggcaactcctgggcagggtccagacaggccacaagaagtcagctatacagacactaaagtgattggaaatggatcatttggtgtggtatatcaagccaaactttgtgattcaggagaactggtcgccatcaagaaagtattgcaggacaagagatttaagaatcgagagctccagatcatgagaaagctagatcactgtaacatagtccgattgcgttatttcttctactccagtggtgagaagaaagatgaggtctatcttaatctggtgctggactatgttccggaaacagtatacagagttgccagacactatagtcgagccaaacagacgctccctgtgatttatgtcaagttgtatatgtatcagctgttccgaagtttagcctatatccattcctttggaatctgccatcgggatattaaaccgcagaacctcttgttggatcctgatactgctgtattaaaactctgtgactttggaagtgcaaagcagctggtccgaggagaacccaatgtttcgtatatctgttctcggtactatagggcaccagagttgatctttggagccactgattatacctctagtatagatgtatggtctgctggctgtgtgttggctgagctgttactaggacaaccaatatttccaggggatagtggtgtggatcagttggtagaaataatcaaggtcctgggaactccaacaagggagcaaatcagagaaatgaacccaaactacacagaatttaaattccctcaaattaaggcacatccttggactaaggtcttccgaccccgaactccaccggaggcaattgcactgtgtagccgtctgctggagtatacaccaactgcccgactaacaccactggaagcttgtgcacattcattttttgatgaattacgggacccaaatgtcaaactaccaaatgggcgagacacacctgcactcttcaacttcaccactcaagatgctaatactggagaccgtggacagaccaataatgctgcttctgcatcagcttccaactccacctgaacagtcccgagcagccagctgcacaggaaaaaccaccagttacttgagtgtcactcagcaacactggtcacgtttggaaagaatattaaaaagagaaaaaaatcctgttcattttagtgttcaatttttttattattattgttgttcttatttaaccttgtaaaatatctataaatacaaaccaatttcattgtattctcactttgagggagatccagggggtgggaggggttgtggggagggggaaagcggagcactagaacatacaatctctctcccacgacaatctttttttattaaaagtctgctgttgtatactttaaaaacaggactcctgcctcatgccccttccacaaaagaagaaaacctttttctgtgctgatgggtttttttgaactttgttttcttttaaagtctagtgtgagactttggtatagtgcacagcttgaaattggttgggagcttagcaggtataactcaacggggacttaaatgtcacttgtaaaattaatccatatcttcgggtatttatagacttgcctttggcatgttggtggcaggtgtggcagacaaagaaatgtgtatcattcgtaacccagggaggtcaataaagtttggaactctacagggaagattcttagtagatttgttaaggttttgttttgctctcagttagtgctagtgatgtagaggcttgtacaggaggctgccagaggggaagcagcaagcaagactcaggcacacatgctctacaggtggctctttgtttgcctgaccaaagttctttgcaaatcttagcacagtttcaaactagtgacctgggaggagatggaaggggtgttgagcaggctgagctagctgctgaggtcaaaggctgatgagcccagaggaaggggacaggtcagggatacatctcaccactgtgaataagtttgtccagatttttttctaaagttacttcccttggaaagatacacttgagaggacattgtagttaaataatgtgaactgtaacagtcatctactggtttatttttcatattttttaattgaaaattgagcttgcagaaatagccacattctacacatagttctaattttaaatccaaatctagaatctgtatttaatttgttttttaacctcatgctttttacatttatttattgatgcatgtcagatggtagaaatattaaaaactacacatcagaatgatacagtcacttatacctgctgactttataggaaagctgatgatataaatgtgtgtatatatgttatatatacatatattcaatactgcctttttttttgtctacagtatcaaaattgactggttgaagcatgagaagaatgtttcccccacacccagttaagagtttttgtgtctgttttctttgtgtatcagtgaacgatgttaagaatcagtctctctttttgaagaaaaagcaatattccttggaaagcaaggagaattgaaggactatgtttgccgtgaggaaatagattttcatgactagtttgttttatacttttaaggttggcatctatgtgggccttatatactctaaaatgaactttagtcaccttggtgcttatgggccattacttgacctatgaatctttaaggcacaatcagttgtactttacatttaaagatcacttgagtgatggccgcctttccctcctacccgctccttccccacatgccttccaaggttagctggtaactgtagggctgcagagctgagcccatggttgtgtgtaacttgccctcaccctcctcattgccaccttaggtcactttatgggtctcgtcctccagagggttcggaagtggagtctgttggcagccctcctgcaggccctagcaccctgtcctgctccttaactgtgtgtgtgactctccaagagagttgtcctgcctgctgaagtgaaccagtacccagaaagacaactgtgagccatcttggttttcactcgctgtttagctgaggtcttgggccacaaaaggggtttcacaaacctctggatatatcagagtttatgagaaaggaaacatgctcagtcaaaccaaatcaaacaaattgaattttatgttttataaagtgcttctgaaagctaagatttgaaagaagtctgaaatcaaagtatttggcagcataactccttaaaggtagtggcgttgatagaccattttcagacagaatttataaagaatctgaaaaggcaggtctgtgatagagaaatggacctgcattcagatccaactgcccagcaagcgtttggatgcagacactgctctggacgtggtatactccccagagtccataaaaatcagtgcttattttaggaaacaggttgccccccacaactggggtaaaagaagagagaaaagtcacgcttttctctcatttcattgtgtgtgcatgtgtgcgtgtgtgtgtgtgtgtgtgtgtgctgagatgtgtgatttttctttctcaaggatcatggtgggatcacagaactcttttatacaagtgagatccaggtctctgaatatctttttgtatataataataataaaaagctcctcaccaaattcaagcttgtacattatattttctttctgtgtttttaaatttaagttttattgttttgtatgtaaatatgtggacccaggaactgttattaatgagcaaaaagttactgttcagggcagtgattctgtttaataatcagacaaaatgtagacgagctttttaaagccatatagttttaactctgtacagtaggtaccggcctgtattattgtaacaataactctagcaatgtatagtgtatctatatagtttggagtgccttcgcttccatgtgtttttttttttaatttgttcttttttaaattttaattggtttcctttatccatgtctccctgtccaccccctttccctttgaaataataactcactcataacagtatctttgccccttccacagttaagtttcagtgataccatactcaggagtgggaagaggaaatcatattcgtaatttcatttcgttgaagccctgcctttgttttggttctgaatgtctttcctcctcggtagcagtgagaccggtttcatttcatacttagtccattcagggacttagtgtagcaccagggagccctagagctggaggatatcgaatagattaaattttgctcgtctcttccacaagccctaaccatgggtcttaaaaacagcagattctgggagccttccatgctctctctctctcctcttttatctacttccctcccaaatgagagagtgacagagaattgtttttttataaatcgaagtttcttaatagtatcaggttttgatacgtcagtggtctaaaatgctatagtgcaattactagcagttactgcacggagtgccaccgtgccaatagaggactgttgttttaacaagggaactcttagcccatttcctccctcccgccatctctacccttgctcaatgaaatatcattttaatttcttttaaaaaaaatcagtttaattcttactgtgtgcccaacacgaaggccttttttgaaagaaaaatagaatgttttgcctcaaagtagtccatataaaatgtcttgaatagaagaaaaaactaccaaaccaaaggttactatttttgaaacatcgtgtgttcattccagcaaggcagaagactgcaccttctttccagtgacatgctgtgtcattttttttaagtcctcttaatttttagacacatttttggtttatgttttaacaatgtatgcctaaccagtcatcttgtctgcaccaatgcaaaggtttctgagaggagtattctctatccctgtggatatgaagacactggcatttcatctatttttccctttcctttttaaaggatttaactttggaatcttccaaaggaagtttggccaatgccagatccccaggaatttggggggttttctttcttttcaactgaaattgtatctgattcctactgttcatgttagtgatcatctaatcacagagccaaacacttttctcccctgtgtggaaaagtaggtatgctttacaataaaatctgtcttttctggtagaaacctgagccactgaaaataaaagagacaactagaagcacagtagagtcccagactgagatctacctttgagaggctttgaaagtaatccctggggtttggattattttcacaagggttatgccgttttattcaagtttgttgctccgttttgcacctctgcaataaaagcaaaatgacaaccagtacataaggggttagcttgacaaagtagacttccttgtgttaatttttaagtttttttttccttaactatatctgtctacaggcagatacagatagttgtatgaaaatctgcttgcctgtaaaatttgcatttataaatgtgttgccgatggatcacttgggcctgtacacataccaattagcgtgaccacttccatcttaaaaacaaacctaaaaaacaaaatttattatatatatatatatatatatataaaggactgtgggttgtatacaaactattgcaaacacttgtgcaaatctgtcttgatataaaggaaaagcaaaatctgtataacattattactacttgaatgcctctgtgactgatttttttttcattttaaatataaacttttttgtgaaaagtatgctcaatgttttttttccctttccccattcccttgtaaatacattttgttctatgtgacttggtttggaaatagttaactggtactgtaatttgcattaaataaaaagtaggttagcctggaaatgaaattaaaattcacaagtgtgtggtctttatttcagtacccaaccctcttccttcaccctactattttgccgctgcaatatgtagtcacatcaccatttccattcctctaattagggaaacattaatctttgttatacagaacaagatatcaataccacttcttgttctttccaatgattttattccattgtgtagccccaagaggtgcagcttccaccttggaaacctttggatttgatgtagaggaagctttgcagacactgcttagaaaagaaagaaaacaactctgaaagggacagtttttaaatgtgtataagctgctgtctttgattactgtgttcatgatttggtgtggctgtattttcttttaactttcatcctattagtaatggtctttgggggtctctgtaaaatatatggacaccacgaacagtggggctgtacctcccaggtaaccaacacatgttgtgtttgagtctgctcatttccaatactggatgatgtatgtaaacatgttatgtctcttagtgcaaaaagaaacatcatttttttagggctggctcactctgtcaggcctaatctaaaggctagatataaggtcatgtgactgctgcttcaataaaaacaaatttatattcga

SEQ ID NO: 4 - DNA, 3002 bp, Homo sapiens
agatcaatccaggctccaggagaaagcaggcgggcgggcggagaaaggagaggccgagcggctcaacccgggccgaggctcggggagcggagagtggcgcagcgcccggccgtccggacccgggccgcgagaccccgctcgcccggccactcgtgctcccacacggacgggcgcgccgccaacccggtgctgactgggttacttttttaaacactaggaatggtaatttctactcttctggacttcaaactaagaagttaaagagacttctctgtaaataaacaaatctcttctgctgtccttttgcatttggagacagctttatttcaccatatccaaggagtataactagtgctgtcattatgaatgtgacaagtttattttcctttacaagtccagctgtgaagagacttcttgggtggaaacagggcgatgaagaagaaaaatgggcagagaaagctgttgatgctttggtgaaaaaactgaagaaaaagaaaggtgccatggaggaactggaaaaggccttgagctgcccagggcaaccgagtaactgtgtcaccattccccgctctctggatggcaggctgcaagtctcccaccggaagggactgcctcatgtcatttactgccgtgtgtggcgctggcccgatcttcagagccaccatgaactaaaaccactggaatgctgtgagtttccttttggttccaagcagaaggaggtctgcatcaatccctaccactataagagagtagaaagccctgtacttcctcctgtgctggttccaagacacagcgaatataatcctcagcacagcctcttagctcagttccgtaacttaggacaaaatgagcctcacatgccactcaacgccacttttccagattctttccagcaacccaacagccacccgtttcctcactctcccaatagcagttacccaaactctcctgggagcagcagcagcacctaccctcactctcccaccagctcagacccaggaagccctttccagatgccagctgatacgcccccacctgcttacctgcctcctgaagaccccatgacccaggatggctctcagccgatggacacaaacatgatggcgcctcccctgccctcagaaatcaacagaggagatgttcaggcggttgcttatgaggaaccaaaacactggtgctctattgtctactatgagctcaacaatcgtgtgggtgaagcgttccatgcctcctccacaagtgtgttggtggatggtttcactgatccttccaacaataagaaccgtttctgccttgggctgctctccaatgttaaccggaattccactattgaaaacaccaggcggcatattggaaaaggagttcatctttattatgttggaggggaggtgtatgccgaatgccttagtgacagtagcatctttgtgcaaagtcggaactgcaactaccatcatggatttcatcctactactgtttgcaagatccctagtgggtgtagtctgaaaatttttaacaaccaagaatttgctcagttattggcacagtctgtgaaccatggatttgagacagtctatgagcttacaaaaatgtgtactatacgtatgagctttgtgaagggctggggagcagaataccaccgccaggatgttactagcaccccctgctggattgagatacatctgcacggccccctccagtggctggataaagttcttactcaaatgggttcacctcataatcctatttcatctgtatcttaaatggccccaggcatctgcctctggaaaactattgagccttgcatgtacttgaaggatggatgagtcagacacgattgagaactgacaaaggagccttgataatacttgacctctgtgaccaactgttggattcagaaatttaaacaaaaaaaaaaaaaaacacacacaccttggtaacatactgttgatatcaagaacctgtttagtttacattgtaacattctattgtaaaatcaactaaaattcagacttttagcaggactttgtgtacagttaaaggagagatggccaagccagggacaaattgtctattagaaaacggtcctaagagattctttggtgtttggcactttaaggtcatcgttgggcagaagtttagcattaatagttgttctgaaacgtgttttatcaggtttagagcccatgttgagtcttcttttcatgggttttcataatattttaaaactatttgtttagcgatggttttgttcgtttaagtaaaggttaatcttgatgatatacataataatctttctaaaattgtatgctgaccatacttgctgtcagaataatgctaggcatatgctttttgctaaatatgtatgtacagagtatttggaagttaagaattgattagactagtgaatttaggagtatttgaggtgggtggggggaagagggaaatgacaactgcaaatgtagactatactgtaaaaattcagtttgttgctttaaagaaacaaactgatacctgaattttgctgtgtttccattttttagagatttttatcatttttttctctctcggcattcttttttctcatactcttcaaaaagcagttctgcagctggttaattcatgtaactgtgagagcaaatgaataattcctgctattctgaaattgcctacatgtttcaataccagttatatggagtgcttgaatttaataagcagtttttacggagtttacagtacagaaataggctttaattttcaagtgaattttttgccaaacttagtaactctgttaaatatttggaggatttaaagaacatcccagtttgaattcatttcaaactttttaaatttttttgtactatgtttggttttattttccttctgttaatcttttgtattcacttatgctctcgtacattgagtacttttattccaaaactagtgggttttctctactggaaattttcaataaacctgtcattattgcttactttgattaaaaa

SEQ ID NO: 5 - DNA, 2880 bp, Homo sapiens
cactgcatgtgtattcgtgagttcgcggttgaacaactgttcctttactctgctccctgtctttgtgctgactgggttacttttttaaacactaggaatggtaatttctactcttctggacttcaaactaagaagttaaagagacttctctgtaaataaacaaatctcttctgctgtccttttgcatttggagacagctttatttcaccatatccaaggagtataactagtgctgtcattatgaatgtgacaagtttattttcctttacaagtccagctgtgaagagacttcttgggtggaaacagggcgatgaagaagaaaaatgggcagagaaagctgttgatgctttggtgaaaaaactgaagaaaaagaaaggtgccatggaggaactggaaaaggccttgagctgcccagggcaaccgagtaactgtgtcaccattccccgctctctggatggcaggctgcaagtctcccaccggaagggactgcctcatgtcatttactgccgtgtgtggcgctggcccgatcttcagagccaccatgaactaaaaccactggaatgctgtgagtttccttttggttccaagcagaaggaggtctgcatcaatccctaccactataagagagtagaaagccctgtacttcctcctgtgctggttccaagacacagcgaatataatcctcagcacagcctcttagctcagttccgtaacttaggacaaaatgagcctcacatgccactcaacgccacttttccagattctttccagcaacccaacagccacccgtttcctcactctcccaatagcagttacccaaactctcctgggagcagcagcagcacctaccctcactctcccaccagctcagacccaggaagccctttccagatgccagctgatacgcccccacctgcttacctgcctcctgaagaccccatgacccaggatggctctcagccgatggacacaaacatgatggcgcctcccctgccctcagaaatcaacagaggagatgttcaggcggttgcttatgaggaaccaaaacactggtgctctattgtctactatgagctcaacaatcgtgtgggtgaagcgttccatgcctcctccacaagtgtgttggtggatggtttcactgatccttccaacaataagaaccgtttctgccttgggctgctctccaatgttaaccggaattccactattgaaaacaccaggcggcatattggaaaaggagttcatctttattatgttggaggggaggtgtatgccgaatgccttagtgacagtagcatctttgtgcaaagtcggaactgcaactaccatcatggatttcatcctactactgtttgcaagatccctagtgggtgtagtctgaaaatttttaacaaccaagaatttgctcagttattggcacagtctgtgaaccatggatttgagacagtctatgagcttacaaaaatgtgtactatacgtatgagctttgtgaagggctggggagcagaataccaccgccaggatgttactagcaccccctgctggattgagatacatctgcacggccccctccagtggctggataaagttcttactcaaatgggttcacctcataatcctatttcatctgtatcttaaatggccccaggcatctgcctctggaaaactattgagccttgcatgtacttgaaggatggatgagtcagacacgattgagaactgacaaaggagccttgataatacttgacctctgtgaccaactgttggattcagaaatttaaacaaaaaaaaaaaaaaacacacacaccttggtaacatactgttgatatcaagaacctgtttagtttacattgtaacattctattgtaaaatcaactaaaattcagacttttagcaggactttgtgtacagttaaaggagagatggccaagccagggacaaattgtctattagaaaacggtcctaagagattctttggtgtttggcactttaaggtcatcgttgggcagaagtttagcattaatagttgttctgaaacgtgttttatcaggtttagagcccatgttgagtcttcttttcatgggttttcataatattttaaaactatttgtttagcgatggttttgttcgtttaagtaaaggttaatcttgatgatatacataataatctttctaaaattgtatgctgaccatacttgctgtcagaataatgctaggcatatgctttttgctaaatatgtatgtacagagtatttggaagttaagaattgattagactagtgaatttaggagtatttgaggtgggtggggggaagagggaaatgacaactgcaaatgtagactatactgtaaaaattcagtttgttgctttaaagaaacaaactgatacctgaattttgctgtgtttccattttttagagatttttatcatttttttctctctcggcattcttttttctcatactcttcaaaaagcagttctgcagctggttaattcatgtaactgtgagagcaaatgaataattcctgctattctgaaattgcctacatgtttcaataccagttatatggagtgcttgaatttaataagcagtttttacggagtttacagtacagaaataggctttaattttcaagtgaattttttgccaaacttagtaactctgttaaatatttggaggatttaaagaacatcccagtttgaattcatttcaaactttttaaatttttttgtactatgtttggttttattttccttctgttaatcttttgtattcacttatgctctcgtacattgagtacttttattccaaaactagtgggttttctctactggaaattttcaataaacctgtcattattgcttactttgattaaaaa

SEQ ID NO: 6 - DNA, 3303 bp, Homo sapiens
gctgtgggaagcccagttcccgggcccccgagcctcggctcccgggcctgaccgcgctgggatctccccggccgcgctccccttccgcgcgctcctcacatctctcccgtgctgccgccgggccgaggcccgttcgcgtggcccgcggacccattgtgtcccccgcgccggcggggcgacccctgcgggagctggaggacgaccgctggcgctgctctccaaggcgcctggtggagcgggtctcgcgggcgggggaccccggcgccccgggcccctccacatcccgcacgggttttcttctcggccccagcaagcctctttggggtcgaggtcaaggaaagttcgcaccgagatcccctctaatttattcaaaggtttggcggcggcgcgtaattttttccccctcttccgcctacacccgctgcgtctcctggtgtctcgttcctttccctttaccggagtcgattgcctcactgcatgtgtattcgtgctgactgggttacttttttaaacactaggaatggtaatttctactcttctggacttcaaactaagaagttaaagagacttctctgtaaataaacaaatctcttctgctgtccttttgcatttggagacagctttatttcaccatatccaaggagtataactagtgctgtcattatgaatgtgacaagtttattttcctttacaagtccagctgtgaagagacttcttgggtggaaacagggcgatgaagaagaaaaatgggcagagaaagctgttgatgctttggtgaaaaaactgaagaaaaagaaaggtgccatggaggaactggaaaaggccttgagctgcccagggcaaccgagtaactgtgtcaccattccccgctctctggatggcaggctgcaagtctcccaccggaagggactgcctcatgtcatttactgccgtgtgtggcgctggcccgatcttcagagccaccatgaactaaaaccactggaatgctgtgagtttccttttggttccaagcagaaggaggtctgcatcaatccctaccactataagagagtagaaagccctgtacttcctcctgtgctggttccaagacacagcgaatataatcctcagcacagcctcttagctcagttccgtaacttaggacaaaatgagcctcacatgccactcaacgccacttttccagattctttccagcaacccaacagccacccgtttcctcactctcccaatagcagttacccaaactctcctgggagcagcagcagcacctaccctcactctcccaccagctcagacccaggaagccctttccagatgccagctgatacgcccccacctgcttacctgcctcctgaagaccccatgacccaggatggctctcagccgatggacacaaacatgatggcgcctcccctgccctcagaaatcaacagaggagatgttcaggcggttgcttatgaggaaccaaaacactggtgctctattgtctactatgagctcaacaatcgtgtgggtgaagcgttccatgcctcctccacaagtgtgttggtggatggtttcactgatccttccaacaataagaaccgtttctgccttgggctgctctccaatgttaaccggaattccactattgaaaacaccaggcggcatattggaaaaggagttcatctttattatgttggaggggaggtgtatgccgaatgccttagtgacagtagcatctttgtgcaaagtcggaactgcaactaccatcatggatttcatcctactactgtttgcaagatccctagtgggtgtagtctgaaaatttttaacaaccaagaatttgctcagttattggcacagtctgtgaaccatggatttgagacagtctatgagcttacaaaaatgtgtactatacgtatgagctttgtgaagggctggggagcagaataccaccgccaggatgttactagcaccccctgctggattgagatacatctgcacggccccctccagtggctggataaagttcttactcaaatgggttcacctcataatcctatttcatctgtatcttaaatggccccaggcatctgcctctggaaaactattgagccttgcatgtacttgaaggatggatgagtcagacacgattgagaactgacaaaggagccttgataatacttgacctctgtgaccaactgttggattcagaaatttaaacaaaaaaaaaaaaaaacacacacaccttggtaacatactgttgatatcaagaacctgtttagtttacattgtaacattctattgtaaaatcaactaaaattcagacttttagcaggactttgtgtacagttaaaggagagatggccaagccagggacaaattgtctattagaaaacggtcctaagagattctttggtgtttggcactttaaggtcatcgttgggcagaagtttagcattaatagttgttctgaaacgtgttttatcaggtttagagcccatgttgagtcttcttttcatgggttttcataatattttaaaactatttgtttagcgatggttttgttcgtttaagtaaaggttaatcttgatgatatacataataatctttctaaaattgtatgctgaccatacttgctgtcagaataatgctaggcatatgctttttgctaaatatgtatgtacagagtatttggaagttaagaattgattagactagtgaatttaggagtatttgaggtgggtggggggaagagggaaatgacaactgcaaatgtagactatactgtaaaaattcagtttgttgctttaaagaaacaaactgatacctgaattttgctgtgtttccattttttagagatttttatcatttttttctctctcggcattcttttttctcatactcttcaaaaagcagttctgcagctggttaattcatgtaactgtgagagcaaatgaataattcctgctattctgaaattgcctacatgtttcaataccagttatatggagtgcttgaatttaataagcagtttttacggagtttacagtacagaaataggctttaattttcaagtgaattttttgccaaacttagtaactctgttaaatatttggaggatttaaagaacatcccagtttgaattcatttcaaactttttaaatttttttgtactatgtttggttttattttccttctgttaatcttttgtattcacttatgctctcgtacattgagtacttttattccaaaactagtgggttttctctactggaaattttcaataaacctgtcattattgcttactttgattaaaaa

SEQ ID NO: 7 - DNA, 2911 bp, Homo sapiens
caattctgggtacgtacaacttctggggcctgcaaattattggagagtgagtgaggggcaacgaaagatagacataaaagggcgcgtctcgaaaggtgctgactgggttacttttttaaacactaggaatggtaatttctactcttctggacttcaaactaagaagttaaagagacttctctgtaaataaacaaatctcttctgctgtccttttgcatttggagacagctttatttcaccatatccaaggagtataactagtgctgtcattatgaatgtgacaagtttattttcctttacaagtccagctgtgaagagacttcttgggtggaaacagggcgatgaagaagaaaaatgggcagagaaagctgttgatgctttggtgaaaaaactgaagaaaaagaaaggtgccatggaggaactggaaaaggccttgagctgcccagggcaaccgagtaactgtgtcaccattccccgctctctggatggcaggctgcaagtctcccaccggaagggactgcctcatgtcatttactgccgtgtgtggcgctggcccgatcttcagagccaccatgaactaaaaccactggaatgctgtgagtttccttttggttccaagcagaaggaggtctgcatcaatccctaccactataagagagtagaaagccctgtacttcctcctgtgctggttccaagacacagcgaatataatcctcagcacagcctcttagctcagttccgtaacttaggacaaaatgagcctcacatgccactcaacgccacttttccagattctttccagcaacccaacagccacccgtttcctcactctcccaatagcagttacccaaactctcctgggagcagcagcagcacctaccctcactctcccaccagctcagacccaggaagccctttccagatgccagctgatacgcccccacctgcttacctgcctcctgaagaccccatgacccaggatggctctcagccgatggacacaaacatgatggcgcctcccctgccctcagaaatcaacagaggagatgttcaggcggttgcttatgaggaaccaaaacactggtgctctattgtctactatgagctcaacaatcgtgtgggtgaagcgttccatgcctcctccacaagtgtgttggtggatggtttcactgatccttccaacaataagaaccgtttctgccttgggctgctctccaatgttaaccggaattccactattgaaaacaccaggcggcatattggaaaaggagttcatctttattatgttggaggggaggtgtatgccgaatgccttagtgacagtagcatctttgtgcaaagtcggaactgcaactaccatcatggatttcatcctactactgtttgcaagatccctagtgggtgtagtctgaaaatttttaacaaccaagaatttgctcagttattggcacagtctgtgaaccatggatttgagacagtctatgagcttacaaaaatgtgtactatacgtatgagctttgtgaagggctggggagcagaataccaccgccaggatgttactagcaccccctgctggattgagatacatctgcacggccccctccagtggctggataaagttcttactcaaatgggttcacctcataatcctatttcatctgtatcttaaatggccccaggcatctgcctctggaaaactattgagccttgcatgtacttgaaggatggatgagtcagacacgattgagaactgacaaaggagccttgataatacttgacctctgtgaccaactgttggattcagaaatttaaacaaaaaaaaaaaaaaacacacacaccttggtaacatactgttgatatcaagaacctgtttagtttacattgtaacattctattgtaaaatcaactaaaattcagacttttagcaggactttgtgtacagttaaaggagagatggccaagccagggacaaattgtctattagaaaacggtcctaagagattctttggtgtttggcactttaaggtcatcgttgggcagaagtttagcattaatagttgttctgaaacgtgttttatcaggtttagagcccatgttgagtcttcttttcatgggttttcataatattttaaaactatttgtttagcgatggttttgttcgtttaagtaaaggttaatcttgatgatatacataataatctttctaaaattgtatgctgaccatacttgctgtcagaataatgctaggcatatgctttttgctaaatatgtatgtacagagtatttggaagttaagaattgattagactagtgaatttaggagtatttgaggtgggtggggggaagagggaaatgacaactgcaaatgtagactatactgtaaaaattcagtttgttgctttaaagaaacaaactgatacctgaattttgctgtgtttccattttttagagatttttatcatttttttctctctcggcattcttttttctcatactcttcaaaaagcagttctgcagctggttaattcatgtaactgtgagagcaaatgaataattcctgctattctgaaattgcctacatgtttcaataccagttatatggagtgcttgaatttaataagcagtttttacggagtttacagtacagaaataggctttaattttcaagtgaattttttgccaaacttagtaactctgttaaatatttggaggatttaaagaacatcccagtttgaattcatttcaaactttttaaatttttttgtactatgtttggttttattttccttctgttaatcttttgtattcacttatgctctcgtacattgagtacttttattccaaaactagtgggttttctctactggaaattttcaataaacctgtcattattgcttactttgattaaaaa

SEQ ID NO: 8 - DNA, 2919 bp, Homo sapiens
gccgtcctccggccccggccgcgctgcgctcacgccggccgggccgggaatttggagaggatccctggtcgcgcggcagcggcggcggcgcgcgggtgagcgggtgctgactgggttacttttttaaacactaggaatggtaatttctactcttctggacttcaaactaagaagttaaagagacttctctgtaaataaacaaatctcttctgctgtccttttgcatttggagacagctttatttcaccatatccaaggagtataactagtgctgtcattatgaatgtgacaagtttattttcctttacaagtccagctgtgaagagacttcttgggtggaaacagggcgatgaagaagaaaaatgggcagagaaagctgttgatgctttggtgaaaaaactgaagaaaaagaaaggtgccatggaggaactggaaaaggccttgagctgcccagggcaaccgagtaactgtgtcaccattccccgctctctggatggcaggctgcaagtctcccaccggaagggactgcctcatgtcatttactgccgtgtgtggcgctggcccgatcttcagagccaccatgaactaaaaccactggaatgctgtgagtttccttttggttccaagcagaaggaggtctgcatcaatccctaccactataagagagtagaaagccctgtacttcctcctgtgctggttccaagacacagcgaatataatcctcagcacagcctcttagctcagttccgtaacttaggacaaaatgagcctcacatgccactcaacgccacttttccagattctttccagcaacccaacagccacccgtttcctcactctcccaatagcagttacccaaactctcctgggagcagcagcagcacctaccctcactctcccaccagctcagacccaggaagccctttccagatgccagctgatacgcccccacctgcttacctgcctcctgaagaccccatgacccaggatggctctcagccgatggacacaaacatgatggcgcctcccctgccctcagaaatcaacagaggagatgttcaggcggttgcttatgaggaaccaaaacactggtgctctattgtctactatgagctcaacaatcgtgtgggtgaagcgttccatgcctcctccacaagtgtgttggtggatggtttcactgatccttccaacaataagaaccgtttctgccttgggctgctctccaatgttaaccggaattccactattgaaaacaccaggcggcatattggaaaaggagttcatctttattatgttggaggggaggtgtatgccgaatgccttagtgacagtagcatctttgtgcaaagtcggaactgcaactaccatcatggatttcatcctactactgtttgcaagatccctagtgggtgtagtctgaaaatttttaacaaccaagaatttgctcagttattggcacagtctgtgaaccatggatttgagacagtctatgagcttacaaaaatgtgtactatacgtatgagctttgtgaagggctggggagcagaataccaccgccaggatgttactagcaccccctgctggattgagatacatctgcacggccccctccagtggctggataaagttcttactcaaatgggttcacctcataatcctatttcatctgtatcttaaatggccccaggcatctgcctctggaaaactattgagccttgcatgtacttgaaggatggatgagtcagacacgattgagaactgacaaaggagccttgataatacttgacctctgtgaccaactgttggattcagaaatttaaacaaaaaaaaaaaaaaacacacacaccttggtaacatactgttgatatcaagaacctgtttagtttacattgtaacattctattgtaaaatcaactaaaattcagacttttagcaggactttgtgtacagttaaaggagagatggccaagccagggacaaattgtctattagaaaacggtcctaagagattctttggtgtttggcactttaaggtcatcgttgggcagaagtttagcattaatagttgttctgaaacgtgttttatcaggtttagagcccatgttgagtcttcttttcatgggttttcataatattttaaaactatttgtttagcgatggttttgttcgtttaagtaaaggttaatcttgatgatatacataataatctttctaaaattgtatgctgaccatacttgctgtcagaataatgctaggcatatgctttttgctaaatatgtatgtacagagtatttggaagttaagaattgattagactagtgaatttaggagtatttgaggtgggtggggggaagagggaaatgacaactgcaaatgtagactatactgtaaaaattcagtttgttgctttaaagaaacaaactgatacctgaattttgctgtgtttccattttttagagatttttatcatttttttctctctcggcattcttttttctcatactcttcaaaaagcagttctgcagctggttaattcatgtaactgtgagagcaaatgaataattcctgctattctgaaattgcctacatgtttcaataccagttatatggagtgcttgaatttaataagcagtttttacggagtttacagtacagaaataggctttaattttcaagtgaattttttgccaaacttagtaactctgttaaatatttggaggatttaaagaacatcccagtttgaattcatttcaaactttttaaatttttttgtactatgtttggttttattttccttctgttaatcttttgtattcacttatgctctcgtacattgagtacttttattccaaaactagtgggttttctctactggaaattttcaataaacctgtcattattgcttactttgattaaaaa

SEQ ID NO: 9 - DNA, 2911 bp, Homo sapiens
gccgtcctccggccccggccgcgctgcgctcacgccggccgggccgggaatttggagaggatccctggtcgcgcggcagcggcggcggcgcgcgggtgctgactgggttacttttttaaacactaggaatggtaatttctactcttctggacttcaaactaagaagttaaagagacttctctgtaaataaacaaatctcttctgctgtccttttgcatttggagacagctttatttcaccatatccaaggagtataactagtgctgtcattatgaatgtgacaagtttattttcctttacaagtccagctgtgaagagacttcttgggtggaaacagggcgatgaagaagaaaaatgggcagagaaagctgttgatgctttggtgaaaaaactgaagaaaaagaaaggtgccatggaggaactggaaaaggccttgagctgcccagggcaaccgagtaactgtgtcaccattccccgctctctggatggcaggctgcaagtctcccaccggaagggactgcctcatgtcatttactgccgtgtgtggcgctggcccgatcttcagagccaccatgaactaaaaccactggaatgctgtgagtttccttttggttccaagcagaaggaggtctgcatcaatccctaccactataagagagtagaaagccctgtacttcctcctgtgctggttccaagacacagcgaatataatcctcagcacagcctcttagctcagttccgtaacttaggacaaaatgagcctcacatgccactcaacgccacttttccagattctttccagcaacccaacagccacccgtttcctcactctcccaatagcagttacccaaactctcctgggagcagcagcagcacctaccctcactctcccaccagctcagacccaggaagccctttccagatgccagctgatacgcccccacctgcttacctgcctcctgaagaccccatgacccaggatggctctcagccgatggacacaaacatgatggcgcctcccctgccctcagaaatcaacagaggagatgttcaggcggttgcttatgaggaaccaaaacactggtgctctattgtctactatgagctcaacaatcgtgtgggtgaagcgttccatgcctcctccacaagtgtgttggtggatggtttcactgatccttccaacaataagaaccgtttctgccttgggctgctctccaatgttaaccggaattccactattgaaaacaccaggcggcatattggaaaaggagttcatctttattatgttggaggggaggtgtatgccgaatgccttagtgacagtagcatctttgtgcaaagtcggaactgcaactaccatcatggatttcatcctactactgtttgcaagatccctagtgggtgtagtctgaaaatttttaacaaccaagaatttgctcagttattggcacagtctgtgaaccatggatttgagacagtctatgagcttacaaaaatgtgtactatacgtatgagctttgtgaagggctggggagcagaataccaccgccaggatgttactagcaccccctgctggattgagatacatctgcacggccccctccagtggctggataaagttcttactcaaatgggttcacctcataatcctatttcatctgtatcttaaatggccccaggcatctgcctctggaaaactattgagccttgcatgtacttgaaggatggatgagtcagacacgattgagaactgacaaaggagccttgataatacttgacctctgtgaccaactgttggattcagaaatttaaacaaaaaaaaaaaaaaacacacacaccttggtaacatactgttgatatcaagaacctgtttagtttacattgtaacattctattgtaaaatcaactaaaattcagacttttagcaggactttgtgtacagttaaaggagagatggccaagccagggacaaattgtctattagaaaacggtcctaagagattctttggtgtttggcactttaaggtcatcgttgggcagaagtttagcattaatagttgttctgaaacgtgttttatcaggtttagagcccatgttgagtcttcttttcatgggttttcataatattttaaaactatttgtttagcgatggttttgttcgtttaagtaaaggttaatcttgatgatatacataataatctttctaaaattgtatgctgaccatacttgctgtcagaataatgctaggcatatgctttttgctaaatatgtatgtacagagtatttggaagttaagaattgattagactagtgaatttaggagtatttgaggtgggtggggggaagagggaaatgacaactgcaaatgtagactatactgtaaaaattcagtttgttgctttaaagaaacaaactgatacctgaattttgctgtgtttccattttttagagatttttatcatttttttctctctcggcattcttttttctcatactcttcaaaaagcagttctgcagctggttaattcatgtaactgtgagagcaaatgaataattcctgctattctgaaattgcctacatgtttcaataccagttatatggagtgcttgaatttaataagcagtttttacggagtttacagtacagaaataggctttaattttcaagtgaattttttgccaaacttagtaactctgttaaatatttggaggatttaaagaacatcccagtttgaattcatttcaaactttttaaatttttttgtactatgtttggttttattttccttctgttaatcttttgtattcacttatgctctcgtacattgagtacttttattccaaaactagtgggttttctctactggaaattttcaataaacctgtcattattgcttactttgattaaaaa

SEQ ID NO: 10 - DNA, 3190 bp, Homo sapiens
gctgtgggaagcccagttcccgggcccccgagcctcggctcccgggcctgaccgcgctgggatctccccggccgcgctccccttccgcgcgctcctcacatctctcccgtgctgccgccgggccgaggcccgttcgcgtggcccgcggacccattgtgtcccccgcgccggcggggcgacccctgcgggagctggaggacgaccgctggcgctgctctccaaggcgcctggtggagcgggtctcgcgggcgggggaccccggcgccccgggcccctccacatcccgcacgggttttcttctcggccccagcaagcctctttggggtcgaggtcaaggaaagttcgcaccgagatcccctctaatttattcaaaggtgctgactgggttacttttttaaacactaggaatggtaatttctactcttctggacttcaaactaagaagttaaagagacttctctgtaaataaacaaatctcttctgctgtccttttgcatttggagacagctttatttcaccatatccaaggagtataactagtgctgtcattatgaatgtgacaagtttattttcctttacaagtccagctgtgaagagacttcttgggtggaaacagggcgatgaagaagaaaaatgggcagagaaagctgttgatgctttggtgaaaaaactgaagaaaaagaaaggtgccatggaggaactggaaaaggccttgagctgcccagggcaaccgagtaactgtgtcaccattccccgctctctggatggcaggctgcaagtctcccaccggaagggactgcctcatgtcatttactgccgtgtgtggcgctggcccgatcttcagagccaccatgaactaaaaccactggaatgctgtgagtttccttttggttccaagcagaaggaggtctgcatcaatccctaccactataagagagtagaaagccctgtacttcctcctgtgctggttccaagacacagcgaatataatcctcagcacagcctcttagctcagttccgtaacttaggacaaaatgagcctcacatgccactcaacgccacttttccagattctttccagcaacccaacagccacccgtttcctcactctcccaatagcagttacccaaactctcctgggagcagcagcagcacctaccctcactctcccaccagctcagacccaggaagccctttccagatgccagctgatacgcccccacctgcttacctgcctcctgaagaccccatgacccaggatggctctcagccgatggacacaaacatgatggcgcctcccctgccctcagaaatcaacagaggagatgttcaggcggttgcttatgaggaaccaaaacactggtgctctattgtctactatgagctcaacaatcgtgtgggtgaagcgttccatgcctcctccacaagtgtgttggtggatggtttcactgatccttccaacaataagaaccgtttctgccttgggctgctctccaatgttaaccggaattccactattgaaaacaccaggcggcatattggaaaaggagttcatctttattatgttggaggggaggtgtatgccgaatgccttagtgacagtagcatctttgtgcaaagtcggaactgcaactaccatcatggatttcatcctactactgtttgcaagatccctagtgggtgtagtctgaaaatttttaacaaccaagaatttgctcagttattggcacagtctgtgaaccatggatttgagacagtctatgagcttacaaaaatgtgtactatacgtatgagctttgtgaagggctggggagcagaataccaccgccaggatgttactagcaccccctgctggattgagatacatctgcacggccccctccagtggctggataaagttcttactcaaatgggttcacctcataatcctatttcatctgtatcttaaatggccccaggcatctgcctctggaaaactattgagccttgcatgtacttgaaggatggatgagtcagacacgattgagaactgacaaaggagccttgataatacttgacctctgtgaccaactgttggattcagaaatttaaacaaaaaaaaaaaaaaacacacacaccttggtaacatactgttgatatcaagaacctgtttagtttacattgtaacattctattgtaaaatcaactaaaattcagacttttagcaggactttgtgtacagttaaaggagagatggccaagccagggacaaattgtctattagaaaacggtcctaagagattctttggtgtttggcactttaaggtcatcgttgggcagaagtttagcattaatagttgttctgaaacgtgttttatcaggtttagagcccatgttgagtcttcttttcatgggttttcataatattttaaaactatttgtttagcgatggttttgttcgtttaagtaaaggttaatcttgatgatatacataataatctttctaaaattgtatgctgaccatacttgctgtcagaataatgctaggcatatgctttttgctaaatatgtatgtacagagtatttggaagttaagaattgattagactagtgaatttaggagtatttgaggtgggtggggggaagagggaaatgacaactgcaaatgtagactatactgtaaaaattcagtttgttgctttaaagaaacaaactgatacctgaattttgctgtgtttccattttttagagatttttatcatttttttctctctcggcattcttttttctcatactcttcaaaaagcagttctgcagctggttaattcatgtaactgtgagagcaaatgaataattcctgctattctgaaattgcctacatgtttcaataccagttatatggagtgcttgaatttaataagcagtttttacggagtttacagtacagaaataggctttaattttcaagtgaattttttgccaaacttagtaactctgttaaatatttggaggatttaaagaacatcccagtttgaattcatttcaaactttttaaatttttttgtactatgtttggttttattttccttctgttaatcttttgtattcacttatgctctcgtacattgagtacttttattccaaaactagtgggttttctctactggaaattttcaataaacctgtcattattgcttactttgattaaaaa

SEQ ID NO: 11 - DNA, 3188 bp, Homo sapiens
cactgcatgtgtattcgtgagttcgcggttgaacaactgttcctttactctgctccctgtctttgttagtgtttctcggggttgtttctgtaggaaggtgggggtggtgggcgtgagagacagatgtgggcttgtttttctagttgctgaaactgtatgaaggctttaaagggagaacgttttcttgatgtgctttaggaggggaggaggaacaaatgcctgccagatctcacagctacagtagctgagcttttgtttattttgaagagcatgcaatttttaaatacacggtgcaagataaccagtaaaggcgcgttccttctgaaaattgaggccggtctcagaaccatctcctgagaaagcatccttttcgtgctgactgggttacttttttaaacactaggaatggtaatttctactcttctggacttcaaactaagaagttaaagagacttctctgtaaataaacaaatctcttctgctgtccttttgcatttggagacagctttatttcaccatatccaaggagtataactagtgctgtcattatgaatgtgacaagtttattttcctttacaagtccagctgtgaagagacttcttgggtggaaacagggcgatgaagaagaaaaatgggcagagaaagctgttgatgctttggtgaaaaaactgaagaaaaagaaaggtgccatggaggaactggaaaaggccttgagctgcccagggcaaccgagtaactgtgtcaccattccccgctctctggatggcaggctgcaagtctcccaccggaagggactgcctcatgtcatttactgccgtgtgtggcgctggcccgatcttcagagccaccatgaactaaaaccactggaatgctgtgagtttccttttggttccaagcagaaggaggtctgcatcaatccctaccactataagagagtagaaagccctgtacttcctcctgtgctggttccaagacacagcgaatataatcctcagcacagcctcttagctcagttccgtaacttaggacaaaatgagcctcacatgccactcaacgccacttttccagattctttccagcaacccaacagccacccgtttcctcactctcccaatagcagttacccaaactctcctgggagcagcagcagcacctaccctcactctcccaccagctcagacccaggaagccctttccagatgccagctgatacgcccccacctgcttacctgcctcctgaagaccccatgacccaggatggctctcagccgatggacacaaacatgatggcgcctcccctgccctcagaaatcaacagaggagatgttcaggcggttgcttatgaggaaccaaaacactggtgctctattgtctactatgagctcaacaatcgtgtgggtgaagcgttccatgcctcctccacaagtgtgttggtggatggtttcactgatccttccaacaataagaaccgtttctgccttgggctgctctccaatgttaaccggaattccactattgaaaacaccaggcggcatattggaaaaggagttcatctttattatgttggaggggaggtgtatgccgaatgccttagtgacagtagcatctttgtgcaaagtcggaactgcaactaccatcatggatttcatcctactactgtttgcaagatccctagtgggtgtagtctgaaaatttttaacaaccaagaatttgctcagttattggcacagtctgtgaaccatggatttgagacagtctatgagcttacaaaaatgtgtactatacgtatgagctttgtgaagggctggggagcagaataccaccgccaggatgttactagcaccccctgctggattgagatacatctgcacggccccctccagtggctggataaagttcttactcaaatgggttcacctcataatcctatttcatctgtatcttaaatggccccaggcatctgcctctggaaaactattgagccttgcatgtacttgaaggatggatgagtcagacacgattgagaactgacaaaggagccttgataatacttgacctctgtgaccaactgttggattcagaaatttaaacaaaaaaaaaaaaaaacacacacaccttggtaacatactgttgatatcaagaacctgtttagtttacattgtaacattctattgtaaaatcaactaaaattcagacttttagcaggactttgtgtacagttaaaggagagatggccaagccagggacaaattgtctattagaaaacggtcctaagagattctttggtgtttggcactttaaggtcatcgttgggcagaagtttagcattaatagttgttctgaaacgtgttttatcaggtttagagcccatgttgagtcttcttttcatgggttttcataatattttaaaactatttgtttagcgatggttttgttcgtttaagtaaaggttaatcttgatgatatacataataatctttctaaaattgtatgctgaccatacttgctgtcagaataatgctaggcatatgctttttgctaaatatgtatgtacagagtatttggaagttaagaattgattagactagtgaatttaggagtatttgaggtgggtggggggaagagggaaatgacaactgcaaatgtagactatactgtaaaaattcagtttgttgctttaaagaaacaaactgatacctgaattttgctgtgtttccattttttagagatttttatcatttttttctctctcggcattcttttttctcatactcttcaaaaagcagttctgcagctggttaattcatgtaactgtgagagcaaatgaataattcctgctattctgaaattgcctacatgtttcaataccagttatatggagtgcttgaatttaataagcagtttttacggagtttacagtacagaaataggctttaattttcaagtgaattttttgccaaacttagtaactctgttaaatatttggaggatttaaagaacatcccagtttgaattcatttcaaactttttaaatttttttgtactatgtttggttttattttccttctgttaatcttttgtattcacttatgctctcgtacattgagtacttttattccaaaactagtgggttttctctactggaaattttcaataaacctgtcattattgcttactttgattaaaaa

SEQ ID NO: 12 - DNA, 3362 bp, Homo sapiens
aagcctctcggtctgtggcagcagcgttggcccggccccgggagcggagagcgaggggaggcggagacggaggaaggtctgaggagcagcttcagtccccgccgagccgccaccgcaggtcgaggacggtcggactcccgcggcgggaggagcctgttcccctgagggtatttgaagtataccatacaactgttttgaaaatccagcgtggacaatggctactcaagctgatttgatggagttggacatggccatggaaccagacagaaaagcggctgttagtcactggcagcaacagtcttacctggactctggaatccattctggtgccactaccacagctccttctctgagtggtaaaggcaatcctgaggaagaggatgtggatacctcccaagtcctgtatgagtgggaacagggattttctcagtccttcactcaagaacaagtagctgatattgatggacagtatgcaatgactcgagctcagagggtacgagctgctatgttccctgagacattagatgagggcatgcagatcccatctacacagtttgatgctgctcatcccactaatgtccagcgtttggctgaaccatcacagatgctgaaacatgcagttgtaaacttgattaactatcaagatgatgcagaacttgccacacgtgcaatccctgaactgacaaaactgctaaatgacgaggaccaggtggtggttaataaggctgcagttatggtccatcagctttctaaaaaggaagcttccagacacgctatcatgcgttctcctcagatggtgtctgctattgtacgtaccatgcagaatacaaatgatgtagaaacagctcgttgtaccgctgggaccttgcataacctttcccatcatcgtgagggcttactggccatctttaagtctggaggcattcctgccctggtgaaaatgcttggttcaccagtggattctgtgttgttttatgccattacaactctccacaaccttttattacatcaagaaggagctaaaatggcagtgcgtttagctggtgggctgcagaaaatggttgccttgctcaacaaaacaaatgttaaattcttggctattacgacagactgccttcaaattttagcttatggcaaccaagaaagcaagctcatcatactggctagtggtggaccccaagctttagtaaatataatgaggacctatacttacgaaaaactactgtggaccacaagcagagtgctgaaggtgctatctgtctgctctagtaataagccggctattgtagaagctggtggaatgcaagctttaggacttcacctgacagatccaagtcaacgtcttgttcagaactgtctttggactctcaggaatctttcagatgctgcaactaaacaggaagggatggaaggtctccttgggactcttgttcagcttctgggttcagatgatataaatgtggtcacctgtgcagctggaattctttctaacctcacttgcaataattataagaacaagatgatggtctgccaagtgggtggtatagaggctcttgtgcgtactgtccttcgggctggtgacagggaagacatcactgagcctgccatctgtgctcttcgtcatctgaccagccgacaccaagaagcagagatggcccagaatgcagttcgccttcactatggactaccagttgtggttaagctcttacacccaccatcccactggcctctgataaaggctactgttggattgattcgaaatcttgccctttgtcccgcaaatcatgcacctttgcgtgagcagggtgccattccacgactagttcagttgcttgttcgtgcacatcaggatacccagcgccgtacgtccatgggtgggacacagcagcaatttgtggagggggtccgcatggaagaaatagttgaaggttgtaccggagcccttcacatcctagctcgggatgttcacaaccgaattgttatcagaggactaaataccattccattgtttgtgcagctgctttattctcccattgaaaacatccaaagagtagctgcaggggtcctctgtgaacttgctcaggacaaggaagctgcagaagctattgaagctgagggagccacagctcctctgacagagttacttcactctaggaatgaaggtgtggcgacatatgcagctgctgttttgttccgaatgtctgaggacaagccacaagattacaagaaacggctttcagttgagctgaccagctctctcttcagaacagagccaatggcttggaatgagactgctgatcttggacttgatattggtgcccagggagaaccccttggatatcgccaggatgatcctagctatcgttcttttcactctggtggatatggccaggatgccttgggtatggaccccatgatggaacatgagatgggtggccaccaccctggtgctgactatccagttgatgggctgccagatctggggcatgcccaggacctcatggatgggctgcctccaggtgacagcaatcagctggcctggtttgatactgacctgtaaatcatcctttagctgtattgtctgaacttgcattgtgattggcctgtagagttgctgagagggctcgaggggtgggctggtatctcagaaagtgcctgacacactaaccaagctgagtttcctatgggaacaattgaagtaaactttttgttctggtcctttttggtcgaggagtaacaatacaaatggattttgggagtgactcaagaagtgaagaatgcacaagaatggatcacaagatggaatttagcaaaccctagccttgcttgttaaaatttttttttttttttttttaagaatatctgtaatggtactgactttgcttgctttgaagtagctctttttttttttttttttttttttttttgcagtaactgttttttaagtctctcgtagtgttaagttatagtgaatactgctacagcaatttctaatttttaagaattgagtaatggtgtagaacactaattaattcataatcactctaattaattgtaatctgaataaagtgtaacaattgtgtagcctttttgtataaaatagacaaatagaaaatggtccaattagtttcctttttaatatgcttaaaataagcaggtggatctatttcatgtttttgatcaaaaactatttgggatatgtatgggtagggtaaatcagtaagaggtgttatttggaaccttgttttggacagtttaccagttgccttttatcccaaagttgttgtaacctgctgtgatacgatgcttcaagagaaaatgcggttataaaaaatggttcagaattaaacttttaattcatt

SEQ ID NO: 13 - DNA, 19 bp, Artificial Sequence
  Description of Artificial Sequence Synthetic primer
gaaaataacacaggctttg

SEQ ID NO: 14 - DNA, 18 bp, Artificial Sequence
  Description of Artificial Sequence Synthetic primer
gctccccgagcctggatt

SEQ ID NO: 15 - DNA, 19 bp, Artificial Sequence
  Description of Artificial Sequence Synthetic primer
ggacagaggctctcattcc

SEQ ID NO: 16 - DNA, 21 bp, Artificial Sequence
  Description of Artificial Sequence Synthetic primer
caattcttggatctcatctta

SEQ ID NO: 17 - DNA, 20 bp, Artificial Sequence
  Description of Artificial Sequence Synthetic primer
gggtgaccaagcatgctagc

SEQ ID NO: 18 - DNA, 20 bp, Artificial Sequence
  Description of Artificial Sequence Synthetic primer
cctggccacctccatcttgc

SEQ ID NO: 19 - DNA, 20 bp, Artificial Sequence
  Description of Artificial Sequence Synthetic primer
ggagaggccatgttgaggac

SEQ ID NO: 20 - DNA, 21 bp, Artificial Sequence
  Description of Artificial Sequence Synthetic primer
cctagcgtctacactgggtag